(19) United States
(12) Patent Application Publication
(10) Pub. No.: US 2021/0007999 A1
(43) Pub. Date: Jan. 14, 2021

(54) NANOPARTICLE COMPOSITIONS

(71) Applicant: January Therapeutics, Inc., San Diego, CA (US)

(72) Inventors: Raj RAHEJA, San Diego, CA (US); Robin M. JACKMAN, San Diego, CA (US); Jason A. KAHANA, San Diego, CA (US)

(21) Appl. No.: 16/976,906

(22) PCT Filed: Mar. 1, 2019
(86) PCT No.: PCT/US2019/020391

§ 371 Date: Aug. 31, 2020

Related U.S. Application Data
(60) Provisional application No. 62/637,962, filed on Mar. 2, 2018.

(51) Int. Cl. A61K 9/51; A61K 9/19; A61K 9/00; A61K 9/08; A61K 47/02; A61K 47/26; A61K 9/107; A61K 31/7072; A61K 31/708; A61K 31/7076

(52) U.S. Cl. CPC A61K 9/5169; A61K 9/19; A61K 9/0095; A61K 9/08; A61K 47/02; A61K 31/7076; A61K 9/0019; A61K 9/107; A61K 31/7072; A61K 31/708; A61K 47/26

(57) ABSTRACT

This disclosure provides nanoparticle compositions comprising nucleoside or nucleotide prodrugs, their use as medicinal agents, and processes for their preparation. The disclosure also provides for the use of the nanoparticle compositions described herein as medicaments and/or in the manufacture of medicaments for the treatment of a variety of diseases, including cancer and viral infections. Provided in one aspect is a composition comprising nanoparticles, wherein the nanoparticles comprise a prodrug of a nucleoside or nucleotide, and a pharmaceutically acceptable carrier; wherein the pharmaceutically acceptable carrier comprises albumin.

### CROSS-REFERENCE

This application claims benefit of U.S. Provisional Application No. 62/637,962, filed on Mar. 2, 2018, which is herein incorporated by reference in its entirety.

### BACKGROUND

Nucleoside or nucleotide derivatives are widely used in treating cancer or viral infections.

### BRIEF SUMMARY OF THE DISCLOSURE

This disclosure provides, for example, nanoparticle compositions comprising nucleoside or nucleotide prodrugs, their use as medicinal agents, and processes for their preparation. The disclosure also provides for the use of the nanoparticle compositions described herein as medicaments and/or in the manufacture of medicaments for the treatment of a variety of diseases, including cancer and viral infections.

Provided in one aspect is a composition comprising nanoparticles, wherein the nanoparticles comprise a prodrug of a nucleoside or nucleotide, and a pharmaceutically acceptable carrier; wherein the pharmaceutically acceptable carrier comprises albumin.

In some embodiments, the nanoparticles have an average diameter of about 1000 nm or less for at least about 15 minutes after nanoparticle formation. In some embodiments, the nanoparticles have an average diameter of about 10 nm or greater for at least about 15 minutes after nanoparticle formation. In some embodiments, nanoparticles have an average diameter of from about 10 nm to about 1000 nm for at least about 15 minutes after nanoparticle formation. In some embodiments, the nanoparticles have an average diameter of about 1000 nm or less for at least about 4 hours after nanoparticle formation. In some embodiments, the nanoparticles have an average diameter of about 10 nm or greater for at least about 4 hours nanoparticle formation. In some embodiments, nanoparticles have an average diameter of from about 10 nm to about 1000 nm for at least about 4 hours after nanoparticle formation.

In some embodiments, the nanoparticles have an average diameter of from about 10 nm to about 1000 nm. In some embodiments, the nanoparticles have an average diameter of from about 30 nm to about 250 nm. In some embodiments, the nanoparticles have an average diameter of from about 50 nm to about 200 nm. In some embodiments, the nanoparticles have an average diameter of from about 50 nm to about 150 nm. In some embodiments, the nanoparticles have an average diameter of from about 50 nm to about 100 nm. In some embodiments, the albumin is human serum albumin. In some embodiments, the molar ratio of the prodrug of the nucleoside or nucleotide to pharmaceutically acceptable carrier is from about 1:1 to about 20:1. In some embodiments, the molar ratio of the prodrug of the nucleoside or nucleotide to pharmaceutically acceptable carrier is from about 2:1 to about 12:1. In some embodiments, the nanoparticles are suspended, dissolved, or emulsified in a liquid. In some embodiments, the composition is sterile filterable.

In some embodiments, the composition is dehydrated. In some embodiments, the composition is a lyophilized composition. In some embodiments, the composition comprises from about 0.9% to about 24% by weight of the prodrug of the nucleoside or nucleotide. In some embodiments, the composition comprises from about 1.8% to about 16% by weight of the prodrug of the nucleoside or nucleotide. In some embodiments, the composition comprises from about 76% to about 99% by weight of the pharmaceutically acceptable carrier. In some embodiments, the composition comprises from about 84% to about 98% by weight of the pharmaceutically acceptable carrier.

In some embodiments, the composition is reconstituted with an appropriate biocompatible liquid to provide a reconstituted composition. In some embodiments, the appropriate biocompatible liquid is a buffered solution. In some embodiments, the appropriate biocompatible liquid is a solution comprising dextrose. In some embodiments, the appropriate biocompatible liquid is a solution comprising one or more salts. In some embodiments, the appropriate biocompatible liquid is sterile water, saline, phosphate-buffered saline, 5% dextrose in water solution, Ringer's solution, or Ringer's lactate solution. In some embodiments, the nanoparticles have an average diameter of from about 10 nm to about 1000 nm after reconstitution. In some embodiments, the nanoparticles have an average diameter of from about 30 nm to about 250 nm after reconstitution. In some embodiments, the nanoparticles have an average diameter of from about 50 nm to about 200 nm after reconstitution. In some embodiments, the nanoparticles have an average diameter of from about 50 nm to about 150 nm after reconstitution. In some embodiments, the nanoparticles have an average diameter of from about 50 nm to about 100 nm after reconstitution.

In some embodiments, the composition is suitable for injection. In some embodiments, the composition is suitable for intravenous administration. In some embodiments, the composition is administered intraperitoneally, intraarterially, intrapulmonarily, orally, by inhalation, intravesicularly, intramuscularly, intratracheally, subcutaneously, intraocularly, intrathecally, intratumorally, or transdermally.

In some embodiments, the prodrug of the nucleoside or nucleotide is a prodrug of a nucleoside. In some embodiments, the prodrug of the nucleoside or nucleotide is a prodrug of a nucleotide. In some embodiments, the prodrug of the nucleoside or nucleotide comprises a cyclic or acyclic sugar moiety. In some embodiments, the prodrug of the nucleoside or nucleotide comprises a sugar moiety that is an optionally substituted pentose. In some embodiments, the prodrug of the nucleoside or nucleotide comprises a sugar moiety that is a modified pentose moiety. In some embodiments, the prodrug of the nucleoside or nucleotide comprises a nitrogenous base that is an optionally substituted purine-base or an optionally substituted pyrimidine-base. In some embodiments, the prodrug of the nucleoside or nucleotide is derived from a nucleoside or nucleotide that is an anticancer agent. In some embodiments, the prodrug of the nucleoside or nucleotide is derived from a nucleoside or nucleotide that is an antiviral agent.

In some embodiments, the prodrug of the nucleoside or nucleotide is not a gemcitabine derivative having the following structure:

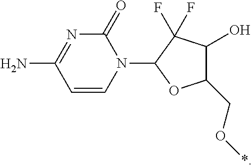

In some embodiments, the prodrug of the nucleoside or a nucleotide is not a compound of Formula (I):

A¹-X¹—X²-A² Formula (I)

wherein:
A¹ is a hydrophilic group;
A² is a gemcitabine moiety;
X¹ is —(CH₂)₁₂—, —(CH₂)₁₄—, —(CH₂)₁₆—, —(CH₂)₁₈—, —(CH₂)₂₀—, or —(CH₂)₂₂—; and
X² is a direct bond or an organic group.

In some embodiments, A¹ is a carboxylic acid group, a carboxylate anion, or a carboxylate ester.

Provided in another aspect is method of treating a disease in a subject in need thereof comprising administering any one of the compositions described herein. In some embodiments, the disease is cancer. In some embodiments, the disease is caused by an infection. In some embodiments, the infection is viral.

Provided in another aspect is method of delivering a prodrug of a nucleoside or nucleotide to a subject in need thereof comprising administering any one of the compositions described herein.

Provided in another aspect is a process of preparing any one of the compositions described herein comprising
a) dissolving a prodrug of a nucleoside or nucleotide in a volatile solvent to form a solution comprising a dissolved prodrug of a nucleoside or nucleotide;
b) adding the solution comprising the dissolved prodrug of a nucleoside or nucleotide to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form any one of the compositions described herein.

In some embodiments, the volatile solvent is a chlorinated solvent, alcohol, ketone, ester, ether, acetonitrile, or any combination thereof. In some embodiments, the volatile solvent is chloroform, ethanol, methanol, or butanol. In some embodiments, the homogenization is high pressure homogenization. In some embodiments, the emulsion is cycled through high pressure homogenization for an appropriate amount of cycles. In some embodiments, the appropriate amount of cycles is from about 2 to about 10 cycles. In some embodiments, the evaporation is accomplished with a rotary evaporator. In some embodiments, the evaporation is under reduced pressure.

### DETAILED DESCRIPTION OF THE DISCLOSURE

This application recognizes that the use of nanoparticles as a drug delivery platform is an attractive approach as nanoparticles provide the following advantages: more specific drug targeting and delivery, reduction in toxicity while maintaining therapeutic effects, greater safety and biocompatibility, and faster development of new safe medicines. The use of a pharmaceutically acceptable carrier, such as a protein, is also advantageous as proteins, such as albumin, are nontoxic, non-immunogenic, biocompatible, and biodegradable.

This application also recognizes that nucleoside or nucleotide derivatives are difficult to formulate into dosage forms that achieve and/or optimize the desired therapeutic effect(s) while minimizing its adverse effects. As such, there exists a need to develop compositions that deliver nucleoside or nucleotide derivatives with improved drug delivery and efficacy.

The application also recognizes that, in a non-limiting example, that chemically modifying a nucleoside or nucleotide into the corresponding prodrug form allows for the formulation of a nanoparticle composition wherein albumin is the carrier. In some instances, as evidenced by the examples demonstrated herein, a wide variety of nucleosides or nucleotides is compatible for use, regardless of nitrogenous base (either natural or non-natural base), ring structure of the sugar moiety (either cyclic or acyclic), and number of phosphate groups (either none or containing at least one phosphate group).

Provided herein are compositions comprising nanoparticles that allow for the drug delivery of nucleoside or nucleotide prodrugs. These nanoparticle compositions further comprise pharmaceutically acceptable carriers that interact with the nucleoside or nucleotide prodrugs to provide the compositions in a form that is suitable for administration to a subject in need thereof. In some embodiments, this application recognizes that the use of nucleoside or nucleotide prodrugs, such as the compounds described herein with specific pharmaceutically acceptable carriers, such as the albumin-based pharmaceutically acceptable carriers described herein, provide nanoparticle formulations that are stable. Also, this application recognizes that, in some instances, use of unmodified nucleoside or nucleotide (e.g. without forming the prodrug as described herein) with the albumin-based pharmaceutically acceptable carriers described herein do not result in stable nanoparticle formulations.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” or “alkylene” refers to a straight or branched hydrocarbon chain radical consisting solely of carbon and hydrogen atoms, containing no unsaturation, having from one to eighteen carbon atoms (e.g., C₁-C₁₈ alkyl). In certain embodiments, an alkyl comprises three to eighteen carbon atoms (e.g., C₃-C₁₈ alkyl). In certain embodiments, an alkyl comprises one to fifteen carbon atoms (e.g., C₁-C₁₅ alkyl). In certain embodiments, an alkyl comprises one to twelve carbon atoms (e.g., C₁-C₁₂ alkyl). In certain embodiments, an alkyl comprises one to eight carbon atoms (e.g., C₁-C₈ alkyl). In other embodiments, an alkyl comprises one to six carbon atoms (e.g., C₁-C₆ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R, —N(R^{a}), —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f}(where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to eighteen carbon atoms. In certain embodiments, an alkenyl comprises three to eighteen carbon atoms. In certain embodiments, an alkenyl comprises three to twelve carbon atoms. In certain embodiments, an alkenyl comprises six to twelve carbon atoms. In certain embodiments, an alkenyl comprises six to ten carbon atoms. In certain embodiments, an alkenyl comprises eight to ten carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR(where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Alkynyl” refers to a straight or branched hydrocarbon chain radical group consisting solely of carbon and hydrogen atoms, containing at least one carbon-carbon triple bond, having from two to eighteen carbon atoms. In certain embodiments, an alkynyl comprises three to eighteen carbon atoms. In certain embodiments, an alkynyl comprises three to twelve carbon atoms. In certain embodiments, an alkynyl comprises six to twelve carbon atoms. In certain embodiments, an alkynyl comprises six to ten carbon atoms. In certain embodiments, an alkynyl comprises eight to ten carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R, —N(R^{a})₂, —C(O)R^{a}, —C(O)OR^{a}, —C(O)N(R^{a})₂, —N(R^{a})C(O)OR, —OC(O)—NR^{a}R^{f}, —N(R^{a})C(O)R, —N(R^{a})S(O)ₜR(where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R—S(O)OR^{a} (where t is 1 or 2), —R^{b}—S(O)ₜR^{a} (where t is 1 or 2), and —R—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“Aryloxy” refers to a radical bonded through an oxygen atom of the formula —O-aryl, where aryl is as defined above.

“Aralkyl” refers to a radical of the formula —R-aryl where R^{C} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkyloxy” refers to a radical bonded through an oxygen atom of the formula —O-aralkyl, where aralkyl is as defined above.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. The cycloalkyl is attached to the rest of the molecule by a single bond. Cycloalkyls are saturated, (i.e., containing single C—C bonds only) or partially unsaturated (i.e., containing one or more double bonds or triple bonds). Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₈ cycloalkyl). In other embodiments, a cycloalkyl comprises three to four carbon atoms (e.g., C₃-C₄ cycloalkyl). A partially unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless stated otherwise specifically in the specification, the term “cycloalkyl” is meant to include cycloalkyl radicals that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R—S(O)ₜOR^{a} (where t is 1 or 2), —R^{b}—S(O)ₜR^{a} (where t is 1 or 2), and —R—S(O)N(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“Halo” or “halogen” refers to bromo, chloro, fluoro or iodo substituents.

“Haloalkyl” refers to an alkyl radical, as defined above, that is substituted by one or more halo radicals, as defined above.

“Haloalkoxy” refers to an alkoxy radical, as defined above, that is substituted by one or more halo radicals, as defined above.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. The alkyl part of the fluoroalkyl radical are optionally substituted as defined above for an alkyl group.

“Heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocycloalkyl radical is a monocyclic, bicyclic, tricyclic, or tetracyclic ring system, which include fused, spiro, or bridged ring systems. The heteroatoms in the heterocycloalkyl radical are optionally oxidized. One or more nitrogen atoms, if present, are optionally quaternized. The heterocycloalkyl radical is partially or fully saturated. In some embodiments, the heterocycloalkyl is attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂—R^{b}—N(R^{a})C(O)OR^{a}, —R^{b}—N(R^{a})C(O)R^{a}, —R^{b}—N(R^{a})S(O)ₜR^{a} (where t is 1 or 2), —R—S(O)ₜOR^{a} (where t is 1 or 2), —R—S(O)R^{a} (where t is 1 or 2), and —R^{b}—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{C} is a straight or branched alkylene or alkenylene chain.

“Heteroaryl” refers to a radical derived from a 3- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above which are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O— heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, the compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans). Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

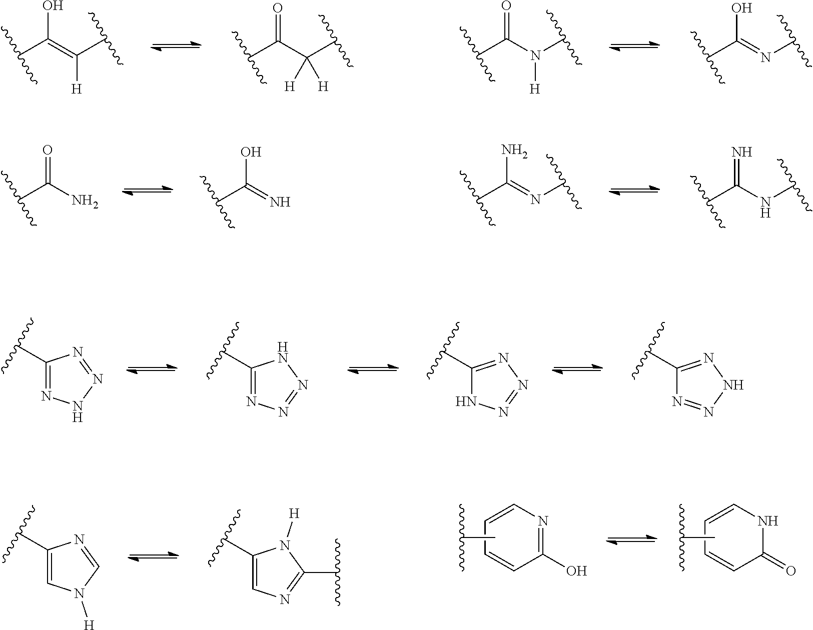

“Optional” or “optionally” means that a subsequently described event or circumstance may or may not occur and that the description includes instances when the event or circumstance occurs and instances in which it does not. For example, “optionally substituted aryl” means that the aryl radical are or are not substituted and that the description includes both substituted aryl radicals and aryl radicals having no substitution.

“Prodrug” is meant to indicate a compound that is converted under physiological conditions or by solvolysis to a biologically active compound described herein. Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility, or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. In some embodiments, prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino, or free mercapto group, respectively. Examples of prodrugs include any suitable derivatives of alcohol or amine functional groups in the active compounds and the like that are known to a skilled practitioner. Examples of any suitable derivatives include but are not limited to acetate, formate, and benzoate derivatives of alcohol or amine functional groups.

As used herein, “treatment” or “treating” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Nucleoside or Nucleotide Prodrugs

In some embodiments, the composition described herein comprises a nucleoside or nucleotide prodrug.

A “nucleoside” refers to a compound comprising a nitrogenous base; and a sugar moiety. A “nucleotide” as used herein refers to a compound comprising a nitrogenous base; a sugar moiety; and at least one phosphate group. In some embodiments, the nucleotide has one phosphate group. In some embodiments, the nucleotide has two phosphate groups. In some embodiments, the nucleotide has three phosphate groups. Nucleosides or nucleotides contemplated for use also include those that are naturally occurring and those that have been synthesized and/or chemically modified.

The compounds described herein are prodrugs that are prepared from nucleosides or nucleotides that are biologically active. Under appropriate conditions upon administration to a subject in need thereof, such as physiological conditions or by solvolysis, these compounds are converted back to the biologically active nucleosides or nucleotides. In some embodiments, the composition described herein comprises a nucleoside prodrug. In some embodiments, the composition described herein comprises a nucleotide prodrug.

In some embodiments, the sugar moiety is an optionally substituted pentose. In some embodiments, the sugar moiety is an optionally substituted ribose or optionally substituted deoxyribose. In some embodiments, the sugar moiety is a modified pentose moiety, wherein an oxygen atom has been replaced with a carbon atom or sulfur atom, or a —CF₂ group; and/or a carbon atom has been replaced with a sulfur or an oxygen atom. In some embodiments, the sugar moiety is a cyclic sugar moiety. In some embodiments, the sugar moiety is an acyclic sugar moiety.

A nitrogenous base refers to an optionally substituted nitrogen-containing heterocycle. In some embodiments, the nitrogenous base is an optionally substituted purine-base, an optionally substituted pyrimidine-base, or an optionally substituted triazole-base (such as, a 1,2,4-triazole). The term “purine-base” is used herein in its ordinary sense as understood by those skilled in the art, and includes its tautomers. Similarly, the term “pyrimidine-base” is used herein in its ordinary sense as understood by those skilled in the art, and includes its tautomers. Examples of purine-bases include, but are not limited to purine, adenine, guanine, hypoxanthine, xanthine, alloxanthine, 7-alkylguanine (e.g., 7-methylguanine), theobromine, caffeine, uric acid and isoguanine. Examples of pyrimidine-bases include, but are not limited to, cytosine, thymine, uracil, 5,6-dihydrouracil, and 5-alkylcytosine (e.g., 5-methylcytosine). An example of a triazole-base is 1,2,4-triazole-3-carboxamide. Other non-limiting examples of nitrogenous bases include diaminopurine, 8-oxo-N⁶-alkyladenine (e.g., 8-oxo-N⁶-methyladenine), 7-deazaxanthine, 7-deazaguanine, 7-deazaadenine, N⁴,N⁴-ethanocytosin, N⁶,N⁶-ethano-2,6-diaminopurine, 5-halouracil (e.g., 5-fluorouracil and 5-bromouracil), pseudoisocytosine, isocytosine, and isoguanine. Nitrogenous bases contemplated for use also include those that are naturally occurring and those that have been synthesized and/or chemically modified.

In some embodiments, the nitrogenous base is an optionally substituted purine-base. In some embodiments, the nitrogenous base is an optionally substituted pyrimidine-base. In some embodiments, the nitrogenous base is an optionally substituted triazole-base. In some embodiments, the nitrogenous base is a naturally occurring nitrogenous base. In some embodiments, the nitrogenous base is a synthetic, non-naturally occurring nitrogenous base. In some embodiments, the nitrogenous base is chemically modified nitrogenous base.

In some embodiments, the prodrug of the nucleoside or nucleotide is not a gemcitabine derivative having the following structure:

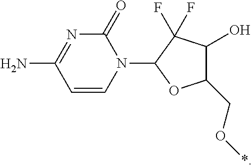

In some embodiments, the prodrug of the nucleoside or a nucleotide is not a compound of Formula (I):

A¹-X¹—X²-A² Formula (I)

wherein:
A¹ is a hydrophilic group;
A² is a gemcitabine moiety;
X¹ is —(CH₂)₁₂—, —(CH₂)₁₄—, —(CH₂)₁₆—, —(CH₂)₁₈—, —(CH₂)₂₀—, or —(CH₂)₂₂—; and
X² is a direct bond or an organic group.

In some embodiments, A¹ is a carboxylic acid group, a carboxylate anion, or a carboxylate ester.

In some embodiments, the prodrug moiety of the nucleoside or nucleotide does not comprise a terminal hydrophilic group. In some embodiments, the prodrug moiety of the nucleoside or nucleotide does not comprise a terminal hydrophilic group selected from a carboxylic acid group, a carboxylate anion, and a carboxylate ester. In some embodiments, the prodrug moiety of the nucleoside or nucleotide does not comprise a terminal hydrophilic group selected from a carboxylic acid group.

### Preparation of Compounds

The compounds used in the reactions described herein are made according to organic synthesis techniques, starting from commercially available chemicals and/or from compounds described in the chemical literature. “Commercially available chemicals” are obtained from standard commercial sources including, but not limited to, Acros Organics (Geel, Belgium), Aldrich Chemical (Milwaukee, Wis., including Sigma Chemical and Fluka), Apin Chemicals Ltd. (Milton Park, UK), Ark Pharm, Inc. (Libertyville, Ill.), Avocado Research (Lancashire, U.K.), BDH Inc. (Toronto, Canada), Bionet (Cornwall, U.K.), Chemservice Inc. (West Chester, Pa.), Combi-blocks (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), Parish Chemical Co. (Orem, Utah), Pfaltz & Bauer, Inc. (Waterbury, CN), Polyorganix (Houston, Tex.), Pierce Chemical Co. (Rockford, Ill.), Riedel de Haen AG (Hanover, Germany), Ryan Scientific, Inc. (Mount Pleasant, S.C.), Spectrum Chemicals (Gardena, Calif.), Sundia Meditech, (Shanghai, China), TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C.). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Prodrugs

In some embodiments, compounds described herein are prodrugs. A “prodrug” refers to an agent that is converted into the parent drug in vivo. Prodrugs are often useful because, in some situations, they are easier to administer than the parent drug. In some embodiments, the prodrug is a substrate for a transporter. In some embodiments, the prodrug also has improved solubility in pharmaceutical compositions over the parent drug. In some embodiments, the design of a prodrug increases the effective water solubility. In some embodiments, the design of a prodrug decreases the effective water solubility. An example, without limitation, of a prodrug is a compound described herein, which is administered as an ester (the “prodrug”) but then is metabolically hydrolyzed to provide the active entity. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the parent compound is incorporated into an acyloxyalkyl ester, alkoxycarbonyloxyalkyl ester, alkyl ester, aryl ester, phosphate ester, sugar ester, ether, and the like.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹C, ¹C, ¹⁵N, ¹⁸O, ¹⁷O, ³¹P, ³²P ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or MeOH. In addition, the compounds provided herein exist in unsolvated as well as solvated forms. In general, the solvated forms are considered equivalent to the unsolvated forms for the purposes of the compounds and methods provided herein.

### Pharmaceutically Acceptable Carrier

In some embodiments, the composition described herein also comprise a pharmaceutically acceptable carrier. In some embodiments, the pharmaceutically acceptable carrier is a protein. The term “protein’ as used herein refers to polypeptides or polymers comprising of amino acids of any length (including full length or fragments). These polypeptides or polymers are linear or branched, comprise modified amino acids, and/or are interrupted by non-amino acids. The term also encompasses an amino acid polymer that has been modified by natural means or by chemical modification. Examples of chemical modifications include, but are not limited to, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The proteins described herein may be naturally occurring, i.e., obtained or derived from a natural source (such as blood), or synthesized (such as chemically synthesized or synthesized by recombinant DNA techniques). In some embodiments, the protein is naturally occurring. In some embodiments, the protein is obtained or derived from a natural source. In some embodiments, the protein is synthetically prepared.

Examples of suitable pharmaceutically acceptable carriers include proteins normally found in blood or plasma, such as albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the pharmaceutically acceptable carrier is a non-blood protein. Examples of non-blood protein include but are not limited to casein, C.-lactalbumin, and B-lactoglobulin.

In some embodiments, the pharmaceutically acceptable carrier is albumin. In some embodiments, the albumin is human serum albumin (HSA). Human serum albumin is the most abundant protein in human blood and is a highly soluble globular protein that consists of 585 amino acids and has a molecular weight of 66.5 kDa. Other albumins suitable for use include, but are not limited to, bovine serum albumin.

In some non-limiting embodiments, the composition described herein further comprises one or more albumin stabilizers. In some embodiments, the albumin stabilizer is N-acetyl tryptophan, octanoate, or a combination thereof.

In some embodiments, the molar ratio of the prodrug of the nucleoside or nucleotide to pharmaceutically acceptable carrier is from about 1:1 to about 40:1. In some embodiments, the molar ratio of the prodrug of the nucleoside or nucleotide to pharmaceutically acceptable carrier is from about 1:1 to about 20:1. In some embodiments, the molar ratio of the prodrug of the nucleoside or nucleotide to pharmaceutically acceptable carrier is from about 2:1 to about 12:1.

In some embodiments, the molar ratio of the prodrug of the nucleoside or nucleotide to pharmaceutically acceptable carrier is about 40:1. In some embodiments, the molar ratio of the prodrug of the nucleoside or nucleotide to pharmaceutically acceptable carrier is about 35:1. In some embodiments, the molar ratio of the prodrug of the nucleoside or nucleotide to pharmaceutically acceptable carrier is about 30:1. In some embodiments, the molar ratio of the prodrug of the nucleoside or nucleotide to pharmaceutically acceptable carrier is about 25:1. In some embodiments, the molar ratio of the prodrug of the nucleoside or nucleotide to pharmaceutically acceptable carrier is about 20:1. In some embodiments, the molar ratio of the prodrug of the nucleoside or nucleotide to pharmaceutically acceptable carrier is about 19:1. In some embodiments, the molar ratio of the prodrug of the nucleoside or nucleotide to pharmaceutically acceptable carrier is about 18:1. In some embodiments, the molar ratio of the prodrug of the nucleoside or nucleotide to pharmaceutically acceptable carrier is about 17:1. In some embodiments, the molar ratio of the prodrug of the nucleoside or nucleotide to pharmaceutically acceptable carrier is about 16:1. In some embodiments, the molar ratio of the prodrug of the nucleoside or nucleotide to pharmaceutically acceptable carrier is about 15:1. In some embodiments, the molar ratio of the prodrug of the nucleoside or nucleotide to pharmaceutically acceptable carrier is about 14:1. In some embodiments, the molar ratio of the prodrug of the nucleoside or nucleotide to pharmaceutically acceptable carrier is about 13:1. In some embodiments, the molar ratio of the prodrug of the nucleoside or nucleotide to pharmaceutically acceptable carrier is about 12:1. In some embodiments, the molar ratio of the prodrug of the nucleoside or nucleotide to pharmaceutically acceptable carrier is about 11:1. In some embodiments, the molar ratio of the prodrug of the nucleoside or nucleotide to pharmaceutically acceptable carrier is about 10:1. In some embodiments, the molar ratio of the prodrug of the nucleoside or nucleotide to pharmaceutically acceptable carrier is about 9:1. In some embodiments, the molar ratio of the prodrug of the nucleoside or nucleotide to pharmaceutically acceptable carrier is about 8:1. In some embodiments, the molar ratio of the prodrug of the nucleoside or nucleotide to pharmaceutically acceptable carrier is about 7:1. In some embodiments, the molar ratio of the prodrug of the nucleoside or nucleotide to pharmaceutically acceptable carrier is about 6:1. In some embodiments, the molar ratio of the prodrug of the nucleoside or nucleotide to pharmaceutically acceptable carrier is about 5:1. In some embodiments, the molar ratio of the prodrug of the nucleoside or nucleotide to pharmaceutically acceptable carrier is about 4:1. In some embodiments, the molar ratio of the prodrug of the nucleoside or nucleotide to pharmaceutically acceptable carrier is about 3:1. In some embodiments, the molar ratio of the prodrug of the nucleoside or nucleotide to pharmaceutically acceptable carrier is about 2:1. In some embodiments, the molar ratio of the prodrug of the nucleoside or nucleotide to pharmaceutically acceptable carrier is about 1:1.

### Nanoparticles

Described herein in one aspect is a composition comprising nanoparticles comprising a prodrug of a nucleoside or nucleotide; and a pharmaceutically acceptable carrier.

In some embodiments, the nanoparticles have an average diameter of about 1000 nm or less for a predetermined amount of time after nanoparticle formation. In some embodiments, the nanoparticles have an average diameter of about 950 nm or less for a predetermined amount of time after nanoparticle formation. In some embodiments, the nanoparticles have an average diameter of about 900 nm or less for a predetermined amount of time after nanoparticle formation. In some embodiments, the nanoparticles have an average diameter of about 850 nm or less for a predetermined amount of time after nanoparticle formation. In some embodiments, the nanoparticles have an average diameter of about 800 nm or less for a predetermined amount of time after nanoparticle formation. In some embodiments, the nanoparticles have an average diameter of about 750 nm or less for a predetermined amount of time after nanoparticle formation. In some embodiments, the nanoparticles have an average diameter of about 700 nm or less for a predetermined amount of time after nanoparticle formation. In some embodiments, the nanoparticles have an average diameter of about 650 nm or less for a predetermined amount of time after nanoparticle formation. In some embodiments, the nanoparticles have an average diameter of about 600 nm or less for a predetermined amount of time after nanoparticle formation. In some embodiments, the nanoparticles have an average diameter of about 550 nm or less for a predetermined amount of time after nanoparticle formation. In some embodiments, the nanoparticles have an average diameter of about 500 nm or less for a predetermined amount of time after nanoparticle formation. In some embodiments, the nanoparticles have an average diameter of about 450 nm or less for a predetermined amount of time after nanoparticle formation. In some embodiments, the nanoparticles have an average diameter of about 400 nm or less for a predetermined amount of time after nanoparticle formation. In some embodiments, the nanoparticles have an average diameter of about 350 nm or less for a predetermined amount of time after nanoparticle formation. In some embodiments, the nanoparticles have an average diameter of about 300 nm or less for a predetermined amount of time after nanoparticle formation. In some embodiments, the nanoparticles have an average diameter of about 250 nm or less for a predetermined amount of time after nanoparticle formation. In some embodiments, the nanoparticles have an average diameter of about 240 nm or less for a predetermined amount of time after nanoparticle formation. In some embodiments, the nanoparticles have an average diameter of about 230 nm or less for a predetermined amount of time after nanoparticle formation. In some embodiments, the nanoparticles have an average diameter of about 220 nm or less for a predetermined amount of time after nanoparticle formation. In some embodiments, the nanoparticles have an average diameter of about 210 nm or less for a predetermined amount of time after nanoparticle formation. In some embodiments, the nanoparticles have an average diameter of about 200 nm or less for a predetermined amount of time after nanoparticle formation. In some embodiments, the nanoparticles have an average diameter of about 190 nm or less for a predetermined amount of time after nanoparticle formation. In some embodiments, the nanoparticles have an average diameter of about 180 nm or less for a predetermined amount of time after nanoparticle formation. In some embodiments, the nanoparticles have an average diameter of about 170 nm or less for a predetermined amount of time after nanoparticle formation. In some embodiments, the nanoparticles have an average diameter of about 160 nm or less for a predetermined amount of time after nanoparticle formation. In some embodiments, the nanoparticles have an average diameter of about 150 nm or less for a predetermined amount of time after nanoparticle formation. In some embodiments, the nanoparticles have an average diameter of about 140 nm or less for a predetermined amount of time after nanoparticle formation. In some embodiments, the nanoparticles have an average diameter of about 130 nm or less for a predetermined amount of time after nanoparticle formation. In some embodiments, the nanoparticles have an average diameter of about 120 nm or less for a predetermined amount of time after nanoparticle formation. In some embodiments, the nanoparticles have an average diameter of about 110 nm or less for a predetermined amount of time after nanoparticle formation. In some embodiments, the nanoparticles have an average diameter of about 100 nm or less for a predetermined amount of time after nanoparticle formation. In some embodiments, the nanoparticles have an average diameter of about 90 nm or less for a predetermined amount of time after nanoparticle formation. In some embodiments, the nanoparticles have an average diameter of about 80 nm or less for a predetermined amount of time after nanoparticle formation. In some embodiments, the nanoparticles have an average diameter of about 70 nm or less for a predetermined amount of time after nanoparticle formation. In some embodiments, the nanoparticles have an average diameter of about 60 nm or less for a predetermined amount of time after nanoparticle formation. In some embodiments, the nanoparticles have an average diameter of about 50 nm or less for a predetermined amount of time after nanoparticle formation. In some embodiments, the nanoparticles have an average diameter of about 40 nm or less for a predetermined amount of time after nanoparticle formation. In some embodiments, the nanoparticles have an average diameter of about 30 nm or less for a predetermined amount of time after nanoparticle formation. In some embodiments, the nanoparticles have an average diameter of about 20 nm or less for a predetermined amount of time after nanoparticle formation. In some embodiments, the nanoparticles have an average diameter of about 10 nm or less for a predetermined amount of time after nanoparticle formation.

In some embodiments, the nanoparticles have an average diameter of about 10 nm or greater for a predetermined amount of time after nanoparticle formation. In some embodiments, the nanoparticles have an average diameter of about 20 nm or greater for a predetermined amount of time after nanoparticle formation. In some embodiments, the nanoparticles have an average diameter of about 30 nm or greater for a predetermined amount of time after nanoparticle formation. In some embodiments, the nanoparticles have an average diameter of about 40 nm or greater for a predetermined amount of time after nanoparticle formation. In some embodiments, the nanoparticles have an average diameter of about 50 nm or greater for a predetermined amount of time after nanoparticle formation. In some embodiments, the nanoparticles have an average diameter of about 60 nm or greater for a predetermined amount of time after nanoparticle formation. In some embodiments, the nanoparticles have an average diameter of about 70 nm or greater for a predetermined amount of time after nanoparticle formation. In some embodiments, the nanoparticles have an average diameter of about 80 nm or greater for a predetermined amount of time after nanoparticle formation. In some embodiments, the nanoparticles have an average diameter of about 90 nm or greater for a predetermined amount of time after nanoparticle formation. In some embodiments, the nanoparticles have an average diameter of about 100 nm or greater for a predetermined amount of time after nanoparticle formation. In some embodiments, the nanoparticles have an average diameter of about 110 nm or greater for a predetermined amount of time after nanoparticle formation. In some embodiments, the nanoparticles have an average diameter of about 120 nm or greater for a predetermined amount of time after nanoparticle formation. In some embodiments, the nanoparticles have an average diameter of about 130 nm or greater for a predetermined amount of time after nanoparticle formation. In some embodiments, the nanoparticles have an average diameter of about 140 nm or greater for a predetermined amount of time after nanoparticle formation. In some embodiments, the nanoparticles have an average diameter of about 150 nm or greater for a predetermined amount of time after nanoparticle formation. In some embodiments, the nanoparticles have an average diameter of about 160 nm or greater for a predetermined amount of time after nanoparticle formation. In some embodiments, the nanoparticles have an average diameter of about 170 nm or greater for a predetermined amount of time after nanoparticle formation. In some embodiments, the nanoparticles have an average diameter of about 180 nm or greater for a predetermined amount of time after nanoparticle formation. In some embodiments, the nanoparticles have an average diameter of about 190 nm or greater for a predetermined amount of time after nanoparticle formation. In some embodiments, the nanoparticles have an average diameter of about 200 nm or greater for a predetermined amount of time after nanoparticle formation. In some embodiments, the nanoparticles have an average diameter of about 210 nm or greater for a predetermined amount of time after nanoparticle formation. In some embodiments, the nanoparticles have an average diameter of about 220 nm or greater for a predetermined amount of time after nanoparticle formation. In some embodiments, the nanoparticles have an average diameter of about 230 nm or greater for a predetermined amount of time after nanoparticle formation. In some embodiments, the nanoparticles have an average diameter of about 240 nm or greater for a predetermined amount of time after nanoparticle formation. In some embodiments, the nanoparticles have an average diameter of about 250 nm or greater for a predetermined amount of time after nanoparticle formation. In some embodiments, the nanoparticles have an average diameter of about 300 nm or greater for a predetermined amount of time after nanoparticle formation. In some embodiments, the nanoparticles have an average diameter of about 350 nm or greater for a predetermined amount of time after nanoparticle formation. In some embodiments, the nanoparticles have an average diameter of about 400 nm or greater for a predetermined amount of time after nanoparticle formation. In some embodiments, the nanoparticles have an average diameter of about 450 nm or greater for a predetermined amount of time after nanoparticle formation. In some embodiments, the nanoparticles have an average diameter of about 500 nm or greater for a predetermined amount of time after nanoparticle formation. In some embodiments, the nanoparticles have an average diameter of about 550 nm or greater for a predetermined amount of time after nanoparticle formation. In some embodiments, the nanoparticles have an average diameter of about 600 nm or greater for a predetermined amount of time after nanoparticle formation. In some embodiments, the nanoparticles have an average diameter of about 650 nm or greater for a predetermined amount of time after nanoparticle formation. In some embodiments, the nanoparticles have an average diameter of about 700 nm or greater for a predetermined amount of time after nanoparticle formation. In some embodiments, the nanoparticles have an average diameter of about 750 nm or greater for a predetermined amount of time after nanoparticle formation. In some embodiments, the nanoparticles have an average diameter of about 800 nm or greater for a predetermined amount of time after nanoparticle formation. In some embodiments, the nanoparticles have an average diameter of about 850 nm or greater for a predetermined amount of time after nanoparticle formation. In some embodiments, the nanoparticles have an average diameter of about 900 nm or greater for a predetermined amount of time after nanoparticle formation. In some embodiments, the nanoparticles have an average diameter of about 950 nm or greater for a predetermined amount of time after nanoparticle formation

In some embodiments, the nanoparticles have an average diameter of from about 10 nm to about 1000 nm for a predetermined amount of time after nanoparticle formation. In some embodiments, the nanoparticles have an average diameter of from about 10 nm to about 950 nm for a predetermined amount of time after nanoparticle formation. In some embodiments, the nanoparticles have an average diameter of from about 10 nm to about 900 nm for a predetermined amount of time after nanoparticle formation. In some embodiments, the nanoparticles have an average diameter of from about 10 nm to about 850 nm for a predetermined amount of time after nanoparticle formation. In some embodiments, the nanoparticles have an average diameter of from about 10 nm to about 800 nm for a predetermined amount of time after nanoparticle formation. In some embodiments, the nanoparticles have an average diameter of from about 10 nm to about 750 nm for a predetermined amount of time after nanoparticle formation. In some embodiments, the nanoparticles have an average diameter of from about 10 nm to about 700 nm for a predetermined amount of time after nanoparticle formation. In some embodiments, the nanoparticles have an average diameter of from about 10 nm to about 650 nm for a predetermined amount of time after nanoparticle formation. In some embodiments, the nanoparticles have an average diameter of from about 10 nm to about 600 nm for a predetermined amount of time after nanoparticle formation. In some embodiments, the nanoparticles have an average diameter of from about 10 nm to about 550 nm for a predetermined amount of time after nanoparticle formation for a predetermined amount of time after nanoparticle formation. In some embodiments, the nanoparticles have an average diameter of from about 10 nm to about 500 nm for a predetermined amount of time after nanoparticle formation. In some embodiments, the nanoparticles have an average diameter of from about 10 nm to about 450 nm for a predetermined amount of time after nanoparticle formation. In some embodiments, the nanoparticles have an average diameter of from about 10 nm to about 400 nm for a predetermined amount of time after nanoparticle formation. In some embodiments, the nanoparticles have an average diameter of from about 10 nm to about 350 nm for a predetermined amount of time after nanoparticle formation. In some embodiments, the nanoparticles have an average diameter of from about 10 nm to about 300 nm for a predetermined amount of time after nanoparticle formation. In some embodiments, the nanoparticles have an average diameter of from about 10 nm to about 250 nm for a predetermined amount of time after nanoparticle formation. In some embodiments, the nanoparticles have an average diameter of from about 10 nm to about 240 nm for a predetermined amount of time after nanoparticle formation. In some embodiments, the nanoparticles have an average diameter of from about 10 nm to about 230 nm for a predetermined amount of time after nanoparticle formation. In some embodiments, the nanoparticles have an average diameter of from about 10 nm to about 220 nm for a predetermined amount of time after nanoparticle formation. In some embodiments, the nanoparticles have an average diameter of from about 10 nm to about 210 nm for a predetermined amount of time after nanoparticle formation. In some embodiments, the nanoparticles have an average diameter of from about 10 nm to about 200 nm for a predetermined amount of time after nanoparticle formation. In some embodiments, the nanoparticles have an average diameter of from about 10 nm to about 190 nm for a predetermined amount of time after nanoparticle formation. In some embodiments, the nanoparticles have an average diameter of from about 10 nm to about 180 nm for a predetermined amount of time after nanoparticle formation. In some embodiments, the nanoparticles have an average diameter of from about 10 nm to about 170 nm for a predetermined amount of time after nanoparticle formation. In some embodiments, the nanoparticles have an average diameter of from about 10 nm to about 160 nm for a predetermined amount of time after nanoparticle formation. In some embodiments, the nanoparticles have an average diameter of from about 10 nm to about 150 nm for a predetermined amount of time after nanoparticle formation. In some embodiments, the nanoparticles have an average diameter of from about 10 nm to about 140 nm for a predetermined amount of time after nanoparticle formation. In some embodiments, the nanoparticles have an average diameter of from about 10 nm to about 130 nm for a predetermined amount of time after nanoparticle formation. In some embodiments, the nanoparticles have an average diameter of from about 10 nm to about 120 nm for a predetermined amount of time after nanoparticle formation. In some embodiments, the nanoparticles have an average diameter of from about 10 nm to about 110 nm for a predetermined amount of time after nanoparticle formation. In some embodiments, the nanoparticles have an average diameter of from about 10 nm to about 100 nm for a predetermined amount of time after nanoparticle formation. In some embodiments, the nanoparticles have an average diameter of from about 10 nm to about 90 nm for a predetermined amount of time after nanoparticle formation. In some embodiments, the nanoparticles have an average diameter of from about 10 nm to about 80 nm for a predetermined amount of time after nanoparticle formation. In some embodiments, the nanoparticles have an average diameter of from about 10 nm to about 70 nm for a predetermined amount of time after nanoparticle formation. In some embodiments, the nanoparticles have an average diameter of from about 10 nm to about 60 nm for a predetermined amount of time after nanoparticle formation. In some embodiments, the nanoparticles have an average diameter of from about 10 nm to about 50 nm for a predetermined amount of time after nanoparticle formation. In some embodiments, the nanoparticles have an average diameter of from about 10 nm to about 40 nm for a predetermined amount of time after nanoparticle formation. In some embodiments, the nanoparticles have an average diameter of from about 10 nm to about 30 nm for a predetermined amount of time after nanoparticle formation. In some embodiments, the nanoparticles have an average diameter of from about 10 nm to about 20 nm for a predetermined amount of time after nanoparticle formation.

In some embodiments, the nanoparticles have an average diameter of about 10 nm for a predetermined amount of time after nanoparticle formation. In some embodiments, the nanoparticles have an average diameter of about 20 nm for a predetermined amount of time after nanoparticle formation. In some embodiments, the nanoparticles have an average diameter of about 30 nm for a predetermined amount of time after nanoparticle formation. In some embodiments, the nanoparticles have an average diameter of about 40 nm for a predetermined amount of time after nanoparticle formation. In some embodiments, the nanoparticles have an average diameter of about 50 nm for a predetermined amount of time after nanoparticle formation. In some embodiments, the nanoparticles have an average diameter of about 60 nm for a predetermined amount of time after nanoparticle formation. In some embodiments, the nanoparticles have an average diameter of about 70 nm for a predetermined amount of time after nanoparticle formation. In some embodiments, the nanoparticles have an average diameter of about 80 nm for a predetermined amount of time after nanoparticle formation. In some embodiments, the nanoparticles have an average diameter of about 90 nm for a predetermined amount of time after nanoparticle formation. In some embodiments, the nanoparticles have an average diameter of about 100 nm for a predetermined amount of time after nanoparticle formation. In some embodiments, the nanoparticles have an average diameter of about 110 nm for a predetermined amount of time after nanoparticle formation. In some embodiments, the nanoparticles have an average diameter of about 120 nm for a predetermined amount of time after nanoparticle formation. In some embodiments, the nanoparticles have an average diameter of about 130 nm for a predetermined amount of time after nanoparticle formation. In some embodiments, the nanoparticles have an average diameter of about 140 nm for a predetermined amount of time after nanoparticle formation. In some embodiments, the nanoparticles have an average diameter of about 150 nm for a predetermined amount of time after nanoparticle formation. In some embodiments, the nanoparticles have an average diameter of about 160 nm for a predetermined amount of time after nanoparticle formation. In some embodiments, the nanoparticles have an average diameter of about 170 nm for a predetermined amount of time after nanoparticle formation. In some embodiments, the nanoparticles have an average diameter of about 180 nm for a predetermined amount of time after nanoparticle formation. In some embodiments, the nanoparticles have an average diameter of about 190 nm for a predetermined amount of time after nanoparticle formation. In some embodiments, the nanoparticles have an average diameter of about 200 nm for a predetermined amount of time after nanoparticle formation. In some embodiments, the nanoparticles have an average diameter of about 210 nm for a predetermined amount of time after nanoparticle formation. In some embodiments, the nanoparticles have an average diameter of about 220 nm for a predetermined amount of time after nanoparticle formation. In some embodiments, the nanoparticles have an average diameter of about 230 nm for a predetermined amount of time after nanoparticle formation. In some embodiments, the nanoparticles have an average diameter of about 240 nm for a predetermined amount of time after nanoparticle formation. In some embodiments, the nanoparticles have an average diameter of about 250 nm for a predetermined amount of time after nanoparticle formation. In some embodiments, the nanoparticles have an average diameter of about 300 nm for a predetermined amount of time after nanoparticle formation. In some embodiments, the nanoparticles have an average diameter of about 350 nm for a predetermined amount of time after nanoparticle formation. In some embodiments, the nanoparticles have an average diameter of about 400 nm for a predetermined amount of time after nanoparticle formation. In some embodiments, the nanoparticles have an average diameter of about 450 nm for a predetermined amount of time after nanoparticle formation. In some embodiments, the nanoparticles have an average diameter of about 500 nm for a predetermined amount of time after nanoparticle formation. In some embodiments, the nanoparticles have an average diameter of about 550 nm for a predetermined amount of time after nanoparticle formation. In some embodiments, the nanoparticles have an average diameter of about 600 nm for a predetermined amount of time after nanoparticle formation. In some embodiments, the nanoparticles have an average diameter of about 650 nm for a predetermined amount of time after nanoparticle formation. In some embodiments, the nanoparticles have an average diameter of about 700 nm for a predetermined amount of time after nanoparticle formation. In some embodiments, the nanoparticles have an average diameter of about 750 nm for a predetermined amount of time after nanoparticle formation. In some embodiments, the nanoparticles have an average diameter of about 800 nm for a predetermined amount of time after nanoparticle formation. In some embodiments, the nanoparticles have an average diameter of about 850 nm for a predetermined amount of time after nanoparticle formation. In some embodiments, the nanoparticles have an average diameter of about 900 nm for a predetermined amount of time after nanoparticle formation. In some embodiments, the nanoparticles have an average diameter of about 950 nm for a predetermined amount of time after nanoparticle formation. In some embodiments, the nanoparticles have an average diameter of about 1000 nm for a predetermined amount of time after nanoparticle formation.

In some embodiments, the predetermined amount of time is at least about 15 minutes. In some embodiments, the predetermined amount of time is at least about 30 minutes. In some embodiments, the predetermined amount of time is at least about 45 minutes. In some embodiments, the predetermined amount of time is at least about 1 hour. In some embodiments, the predetermined amount of time is at least about 2 hours. In some embodiments, the predetermined amount of time is at least about 3 hours. In some embodiments, the predetermined amount of time is at least about 4 hours. In some embodiments, the predetermined amount of time is at least about 5 hours. In some embodiments, the predetermined amount of time is at least about 6 hours. In some embodiments, the predetermined amount of time is at least about 7 hours. In some embodiments, the predetermined amount of time is at least about 8 hours. In some embodiments, the predetermined amount of time is at least about 9 hours. In some embodiments, the predetermined amount of time is at least about 10 hours. In some embodiments, the predetermined amount of time is at least about 11 hours. In some embodiments, the predetermined amount of time is at least about 12 hours. In some embodiments, the predetermined amount of time is at least about 1 day. In some embodiments, the predetermined amount of time is at least about 2 days. In some embodiments, the predetermined amount of time is at least about 3 days. In some embodiments, the predetermined amount of time is at least about 4 days. In some embodiments, the predetermined amount of time is at least about 5 days. In some embodiments, the predetermined amount of time is at least about 6 days. In some embodiments, the predetermined amount of time is at least about 7 days. In some embodiments, the predetermined amount of time is at least about 14 days. In some embodiments, the predetermined amount of time is at least about 21 days. In some embodiments, the predetermined amount of time is at least about 30 days.

In some embodiments, the predetermined amount of time is from about 15 minutes to about 30 days. In some embodiments, the predetermined amount of time is about 30 minutes to about 30 days. In some embodiments, the predetermined amount of time is from about 45 minutes to about 30 days. In some embodiments, the predetermined amount of time is from about 1 hour to about 30 days. In some embodiments, the predetermined amount of time is from about 2 hours to about 30 days. In some embodiments, the predetermined amount of time is from about 3 hours to about 30 days. In some embodiments, the predetermined amount of time is from about 4 hours to about 30 days. In some embodiments, the predetermined amount of time is from about 5 hours to about 30 days. In some embodiments, the predetermined amount of time is from about 6 hours to about 30 days. In some embodiments, the predetermined amount of time is from about 7 hours to about 30 days. In some embodiments, the predetermined amount of time is from about 8 hours to about 30 days. In some embodiments, the predetermined amount of time is from about 9 hours to about 30 days. In some embodiments, the predetermined amount of time is from about 10 hours to about 30 days. In some embodiments, the predetermined amount of time is from about 11 hours to about 30 days. In some embodiments, the predetermined amount of time is from about 12 hours to about 30 days. In some embodiments, the predetermined amount of time is from about 1 day to about 30 days. In some embodiments, the predetermined amount of time is from about 2 days to about 30 days. In some embodiments, the predetermined amount of time is from about 3 days to about 30 days. In some embodiments, the predetermined amount of time is from about 4 days to about 30 days. In some embodiments, the predetermined amount of time is from about 5 days to about 30 days. In some embodiments, the predetermined amount of time is from about 6 days to about 30 days. In some embodiments, the predetermined amount of time is from about 7 days to about 30 days. In some embodiments, the predetermined amount of time is from about 14 days to about 30 days. In some embodiments, the predetermined amount of time is from about 21 days to about 30 days.

In some embodiments, the nanoparticles have an average diameter of about 1000 nm or less for at least about 15 minutes after nanoparticle formation. In some embodiments, the nanoparticles have an average diameter of about 950 nm or less for at least about 15 minutes after nanoparticle formation. In some embodiments, the nanoparticles have an average diameter of about 900 nm or less for at least about 15 minutes after nanoparticle formation. In some embodiments, the nanoparticles have an average diameter of about 850 nm or less for at least about 15 minutes after nanoparticle formation. In some embodiments, the nanoparticles have an average diameter of about 800 nm or less for at least about 15 minutes after nanoparticle formation. In some embodiments, the nanoparticles have an average diameter of about 750 nm or less for at least about 15 minutes after nanoparticle formation. In some embodiments, the nanoparticles have an average diameter of about 700 nm or less for at least about 15 minutes after nanoparticle formation. In some embodiments, the nanoparticles have an average diameter of about 650 nm or less for at least about 15 minutes after nanoparticle formation. In some embodiments, the nanoparticles have an average diameter of about 600 nm or less for at least about 15 minutes after nanoparticle formation. In some embodiments, the nanoparticles have an average diameter of about 550 nm or less for at least about 15 minutes after nanoparticle formation. In some embodiments, the nanoparticles have an average diameter of about 500 nm or less for at least about 15 minutes after nanoparticle formation. In some embodiments, the nanoparticles have an average diameter of about 450 nm or less for at least about 15 minutes after nanoparticle formation. In some embodiments, the nanoparticles have an average diameter of about 400 nm or less for at least about 15 minutes after nanoparticle formation. In some embodiments, the nanoparticles have an average diameter of about 350 nm or less for at least about 15 minutes after nanoparticle formation. In some embodiments, the nanoparticles have an average diameter of about 300 nm or less for at least about 15 minutes after nanoparticle formation. In some embodiments, the nanoparticles have an average diameter of about 250 nm or less for at least about 15 minutes after nanoparticle formation. In some embodiments, the nanoparticles have an average diameter of about 240 nm or less for at least about 15 minutes after nanoparticle formation. In some embodiments, the nanoparticles have an average diameter of about 230 nm or less for at least about 15 minutes after nanoparticle formation. In some embodiments, the nanoparticles have an average diameter of about 220 nm or less for at least about 15 minutes after nanoparticle formation. In some embodiments, the nanoparticles have an average diameter of about 210 nm or less for at least about 15 minutes after nanoparticle formation. In some embodiments, the nanoparticles have an average diameter of about 200 nm or less for at least about 15 minutes after nanoparticle formation. In some embodiments, the nanoparticles have an average diameter of about 190 nm or less for at least about 15 minutes after nanoparticle formation. In some embodiments, the nanoparticles have an average diameter of about 180 nm or less for at least about 15 minutes after nanoparticle formation. In some embodiments, the nanoparticles have an average diameter of about 170 nm or less for at least about 15 minutes after nanoparticle formation. In some embodiments, the nanoparticles have an average diameter of about 160 nm or less for at least about 15 minutes after nanoparticle formation. In some embodiments, the nanoparticles have an average diameter of about 150 nm or less for at least about 15 minutes after nanoparticle formation. In some embodiments, the nanoparticles have an average diameter of about 140 nm or less for at least about 15 minutes after nanoparticle formation. In some embodiments, the nanoparticles have an average diameter of about 130 nm or less for at least about 15 minutes after nanoparticle formation. In some embodiments, the nanoparticles have an average diameter of about 120 nm or less for at least about 15 minutes after nanoparticle formation. In some embodiments, the nanoparticles have an average diameter of about 110 nm or less for at least about 15 minutes after nanoparticle formation. In some embodiments, the nanoparticles have an average diameter of about 100 nm or less for at least about 15 minutes after nanoparticle formation. In some embodiments, the nanoparticles have an average diameter of about 90 nm or less for at least about 15 minutes after nanoparticle formation. In some embodiments, the nanoparticles have an average diameter of about 80 nm or less for at least about 15 minutes after nanoparticle formation. In some embodiments, the nanoparticles have an average diameter of about 70 nm or less for at least about 15 minutes after nanoparticle formation. In some embodiments, the nanoparticles have an average diameter of about 60 nm or less for at least about 15 minutes after nanoparticle formation. In some embodiments, the nanoparticles have an average diameter of about 50 nm or less for at least about 15 minutes after nanoparticle formation. In some embodiments, the nanoparticles have an average diameter of about 40 nm or less for at least about 15 minutes after nanoparticle formation. In some embodiments, the nanoparticles have an average diameter of about 30 nm or less for at least about 15 minutes after nanoparticle formation. In some embodiments, the nanoparticles have an average diameter of about 20 nm or less for at least about 15 minutes after nanoparticle formation. In some embodiments, the nanoparticles have an average diameter of about 10 nm or less for at least about 15 minutes after nanoparticle formation.

In some embodiments, the nanoparticles have an average diameter of about 10 nm or greater for at least about 15 minutes after nanoparticle formation. In some embodiments, the nanoparticles have an average diameter of about 20 nm or greater for at least about 15 minutes after nanoparticle formation. In some embodiments, the nanoparticles have an average diameter of about 30 nm or greater for at least about 15 minutes after nanoparticle formation. In some embodiments, the nanoparticles have an average diameter of about 40 nm or greater for at least about 15 minutes after nanoparticle formation. In some embodiments, the nanoparticles have an average diameter of about 50 nm or greater for at least about 15 minutes after nanoparticle formation. In some embodiments, the nanoparticles have an average diameter of about 60 nm or greater for at least about 15 minutes after nanoparticle formation. In some embodiments, the nanoparticles have an average diameter of about 70 nm or greater for at least about 15 minutes after nanoparticle formation. In some embodiments, the nanoparticles have an average diameter of about 80 nm or greater for at least about 15 minutes after nanoparticle formation. In some embodiments, the nanoparticles have an average diameter of about 90 nm or greater for at least about 15 minutes after nanoparticle formation. In some embodiments, the nanoparticles have an average diameter of about 100 nm or greater for at least about 15 minutes after nanoparticle formation. In some embodiments, the nanoparticles have an average diameter of about 110 nm or greater for at least about 15 minutes after nanoparticle formation. In some embodiments, the nanoparticles have an average diameter of about 120 nm or greater for at least about 15 minutes after nanoparticle formation. In some embodiments, the nanoparticles have an average diameter of about 130 nm or greater for at least about 15 minutes after nanoparticle formation. In some embodiments, the nanoparticles have an average diameter of about 140 nm or greater for at least about 15 minutes after nanoparticle formation. In some embodiments, the nanoparticles have an average diameter of about 150 nm or greater for at least about 15 minutes after nanoparticle formation. In some embodiments, the nanoparticles have an average diameter of about 160 nm or greater for at least about 15 minutes after nanoparticle formation. In some embodiments, the nanoparticles have an average diameter of about 170 nm or greater for at least about 15 minutes after nanoparticle formation. In some embodiments, the nanoparticles have an average diameter of about 180 nm or greater for at least about 15 minutes after nanoparticle formation. In some embodiments, the nanoparticles have an average diameter of about 190 nm or greater for at least about 15 minutes after nanoparticle formation. In some embodiments, the nanoparticles have an average diameter of about 200 nm or greater for at least about 15 minutes after nanoparticle formation. In some embodiments, the nanoparticles have an average diameter of about 210 nm or greater for at least about 15 minutes after nanoparticle formation. In some embodiments, the nanoparticles have an average diameter of about 220 nm or greater for at least about 15 minutes after nanoparticle formation. In some embodiments, the nanoparticles have an average diameter of about 230 nm or greater for at least about 15 minutes after nanoparticle formation. In some embodiments, the nanoparticles have an average diameter of about 240 nm or greater for at least about 15 minutes after nanoparticle formation. In some embodiments, the nanoparticles have an average diameter of about 250 nm or greater for at least about 15 minutes after nanoparticle formation. In some embodiments, the nanoparticles have an average diameter of about 300 nm or greater for at least about 15 minutes after nanoparticle formation. In some embodiments, the nanoparticles have an average diameter of about 350 nm or greater for at least about 15 minutes after nanoparticle formation. In some embodiments, the nanoparticles have an average diameter of about 400 nm or greater for at least about 15 minutes after nanoparticle formation. In some embodiments, the nanoparticles have an average diameter of about 450 nm or greater for at least about 15 minutes after nanoparticle formation. In some embodiments, the nanoparticles have an average diameter of about 500 nm or greater for at least about 15 minutes after nanoparticle formation. In some embodiments, the nanoparticles have an average diameter of about 550 nm or greater for at least about 15 minutes after nanoparticle formation. In some embodiments, the nanoparticles have an average diameter of about 600 nm or greater for at least about 15 minutes after nanoparticle formation. In some embodiments, the nanoparticles have an average diameter of about 650 nm or greater for at least about 15 minutes after nanoparticle formation. In some embodiments, the nanoparticles have an average diameter of about 700 nm or greater for at least about 15 minutes after nanoparticle formation. In some embodiments, the nanoparticles have an average diameter of about 750 nm or greater for at least about 15 minutes after nanoparticle formation. In some embodiments, the nanoparticles have an average diameter of about 800 nm or greater for at least about 15 minutes after nanoparticle formation. In some embodiments, the nanoparticles have an average diameter of about 850 nm or greater for at least about 15 minutes after nanoparticle formation. In some embodiments, the nanoparticles have an average diameter of about 900 nm or greater for at least about 15 minutes after nanoparticle formation. In some embodiments, the nanoparticles have an average diameter of about 950 nm or greater for at least about 15 minutes after nanoparticle formation

In some embodiments, the nanoparticles have an average diameter of from about 10 nm to about 1000 nm for at least about 15 minutes after nanoparticle formation. In some embodiments, the nanoparticles have an average diameter of from about 10 nm to about 950 nm for at least about 15 minutes after nanoparticle formation. In some embodiments, the nanoparticles have an average diameter of from about 10 nm to about 900 nm for at least about 15 minutes after nanoparticle formation. In some embodiments, the nanoparticles have an average diameter of from about 10 nm to about 850 nm for at least about 15 minutes after nanoparticle formation. In some embodiments, the nanoparticles have an average diameter of from about 10 nm to about 800 nm for at least about 15 minutes after nanoparticle formation. In some embodiments, the nanoparticles have an average diameter of from about 10 nm to about 750 nm for at least about 15 minutes after nanoparticle formation. In some embodiments, the nanoparticles have an average diameter of from about 10 nm to about 700 nm for at least about 15 minutes after nanoparticle formation. In some embodiments, the nanoparticles have an average diameter of from about 10 nm to about 650 nm for at least about 15 minutes after nanoparticle formation. In some embodiments, the nanoparticles have an average diameter of from about 10 nm to about 600 nm for at least about 15 minutes after nanoparticle formation. In some embodiments, the nanoparticles have an average diameter of from about 10 nm to about 550 nm for at least about 15 minutes after nanoparticle formation for at least about 15 minutes after nanoparticle formation. In some embodiments, the nanoparticles have an average diameter of from about 10 nm to about 500 nm for at least about 15 minutes after nanoparticle formation. In some embodiments, the nanoparticles have an average diameter of from about 10 nm to about 450 nm for at least about 15 minutes after nanoparticle formation. In some embodiments, the nanoparticles have an average diameter of from about 10 nm to about 400 nm for at least about 15 minutes after nanoparticle formation. In some embodiments, the nanoparticles have an average diameter of from about 10 nm to about 350 nm for at least about 15 minutes after nanoparticle formation. In some embodiments, the nanoparticles have an average diameter of from about 10 nm to about 300 nm for at least about 15 minutes after nanoparticle formation. In some embodiments, the nanoparticles have an average diameter of from about 10 nm to about 250 nm for at least about 15 minutes after nanoparticle formation. In some embodiments, the nanoparticles have an average diameter of from about 10 nm to about 240 nm for at least about 15 minutes after nanoparticle formation. In some embodiments, the nanoparticles have an average diameter of from about 10 nm to about 230 nm for at least about 15 minutes after nanoparticle formation. In some embodiments, the nanoparticles have an average diameter of from about 10 nm to about 220 nm for at least about 15 minutes after nanoparticle formation. In some embodiments, the nanoparticles have an average diameter of from about 10 nm to about 210 nm for at least about 15 minutes after nanoparticle formation. In some embodiments, the nanoparticles have an average diameter of from about 10 nm to about 200 nm for at least about 15 minutes after nanoparticle formation. In some embodiments, the nanoparticles have an average diameter of from about 10 nm to about 190 nm for at least about 15 minutes after nanoparticle formation. In some embodiments, the nanoparticles have an average diameter of from about 10 nm to about 180 nm for at least about 15 minutes after nanoparticle formation. In some embodiments, the nanoparticles have an average diameter of from about 10 nm to about 170 nm for at least about 15 minutes after nanoparticle formation. In some embodiments, the nanoparticles have an average diameter of from about 10 nm to about 160 nm for at least about 15 minutes after nanoparticle formation. In some embodiments, the nanoparticles have an average diameter of from about 10 nm to about 150 nm for at least about 15 minutes after nanoparticle formation. In some embodiments, the nanoparticles have an average diameter of from about 10 nm to about 140 nm for at least about 15 minutes after nanoparticle formation. In some embodiments, the nanoparticles have an average diameter of from about 10 nm to about 130 nm for at least about 15 minutes after nanoparticle formation. In some embodiments, the nanoparticles have an average diameter of from about 10 nm to about 120 nm for at least about 15 minutes after nanoparticle formation. In some embodiments, the nanoparticles have an average diameter of from about 10 nm to about 110 nm for at least about 15 minutes after nanoparticle formation. In some embodiments, the nanoparticles have an average diameter of from about 10 nm to about 100 nm for at least about 15 minutes after nanoparticle formation. In some embodiments, the nanoparticles have an average diameter of from about 10 nm to about 90 nm for at least about 15 minutes after nanoparticle formation. In some embodiments, the nanoparticles have an average diameter of from about 10 nm to about 80 nm for at least about 15 minutes after nanoparticle formation. In some embodiments, the nanoparticles have an average diameter of from about 10 nm to about 70 nm for at least about 15 minutes after nanoparticle formation. In some embodiments, the nanoparticles have an average diameter of from about 10 nm to about 60 nm for at least about 15 minutes after nanoparticle formation. In some embodiments, the nanoparticles have an average diameter of from about 10 nm to about 50 nm for at least about 15 minutes after nanoparticle formation. In some embodiments, the nanoparticles have an average diameter of from about 10 nm to about 40 nm for at least about 15 minutes after nanoparticle formation. In some embodiments, the nanoparticles have an average diameter of from about 10 nm to about 30 nm for at least about 15 minutes after nanoparticle formation. In some embodiments, the nanoparticles have an average diameter of from about 10 nm to about 20 nm for at least about 15 minutes after nanoparticle formation.

In some embodiments, the nanoparticles have an average diameter of about 10 nm for at least about 15 minutes after nanoparticle formation. In some embodiments, the nanoparticles have an average diameter of about 20 nm for at least about 15 minutes after nanoparticle formation. In some embodiments, the nanoparticles have an average diameter of about 30 nm for at least about 15 minutes after nanoparticle formation. In some embodiments, the nanoparticles have an average diameter of about 40 nm for at least about 15 minutes after nanoparticle formation. In some embodiments, the nanoparticles have an average diameter of about 50 nm for at least about 15 minutes after nanoparticle formation. In some embodiments, the nanoparticles have an average diameter of about 60 nm for at least about 15 minutes after nanoparticle formation. In some embodiments, the nanoparticles have an average diameter of about 70 nm for at least about 15 minutes after nanoparticle formation. In some embodiments, the nanoparticles have an average diameter of about 80 nm for at least about 15 minutes after nanoparticle formation. In some embodiments, the nanoparticles have an average diameter of about 90 nm for at least about 15 minutes after nanoparticle formation. In some embodiments, the nanoparticles have an average diameter of about 100 nm for at least about 15 minutes after nanoparticle formation. In some embodiments, the nanoparticles have an average diameter of about 110 nm for at least about 15 minutes after nanoparticle formation. In some embodiments, the nanoparticles have an average diameter of about 120 nm for at least about 15 minutes after nanoparticle formation. In some embodiments, the nanoparticles have an average diameter of about 130 nm for at least about 15 minutes after nanoparticle formation. In some embodiments, the nanoparticles have an average diameter of about 140 nm for at least about 15 minutes after nanoparticle formation. In some embodiments, the nanoparticles have an average diameter of about 150 nm for at least about 15 minutes after nanoparticle formation. In some embodiments, the nanoparticles have an average diameter of about 160 nm for at least about 15 minutes after nanoparticle formation. In some embodiments, the nanoparticles have an average diameter of about 170 nm for at least about 15 minutes after nanoparticle formation. In some embodiments, the nanoparticles have an average diameter of about 180 nm for at least about 15 minutes after nanoparticle formation. In some embodiments, the nanoparticles have an average diameter of about 190 nm for at least about 15 minutes after nanoparticle formation. In some embodiments, the nanoparticles have an average diameter of about 200 nm for at least about 15 minutes after nanoparticle formation. In some embodiments, the nanoparticles have an average diameter of about 210 nm for at least about 15 minutes after nanoparticle formation. In some embodiments, the nanoparticles have an average diameter of about 220 nm for at least about 15 minutes after nanoparticle formation. In some embodiments, the nanoparticles have an average diameter of about 230 nm for at least about 15 minutes after nanoparticle formation. In some embodiments, the nanoparticles have an average diameter of about 240 nm for at least about 15 minutes after nanoparticle formation. In some embodiments, the nanoparticles have an average diameter of about 250 nm for at least about 15 minutes after nanoparticle formation. In some embodiments, the nanoparticles have an average diameter of about 300 nm for at least about 15 minutes after nanoparticle formation. In some embodiments, the nanoparticles have an average diameter of about 350 nm for at least about 15 minutes after nanoparticle formation. In some embodiments, the nanoparticles have an average diameter of about 400 nm for at least about 15 minutes after nanoparticle formation. In some embodiments, the nanoparticles have an average diameter of about 450 nm for at least about 15 minutes after nanoparticle formation. In some embodiments, the nanoparticles have an average diameter of about 500 nm for at least about 15 minutes after nanoparticle formation. In some embodiments, the nanoparticles have an average diameter of about 550 nm for at least about 15 minutes after nanoparticle formation. In some embodiments, the nanoparticles have an average diameter of about 600 nm for at least about 15 minutes after nanoparticle formation. In some embodiments, the nanoparticles have an average diameter of about 650 nm for at least about 15 minutes after nanoparticle formation. In some embodiments, the nanoparticles have an average diameter of about 700 nm for at least about 15 minutes after nanoparticle formation. In some embodiments, the nanoparticles have an average diameter of about 750 nm for at least about 15 minutes after nanoparticle formation. In some embodiments, the nanoparticles have an average diameter of about 800 nm for at least about 15 minutes after nanoparticle formation. In some embodiments, the nanoparticles have an average diameter of about 850 nm for at least about 15 minutes after nanoparticle formation. In some embodiments, the nanoparticles have an average diameter of about 900 nm for at least about 15 minutes after nanoparticle formation. In some embodiments, the nanoparticles have an average diameter of about 950 nm for at least about 15 minutes after nanoparticle formation. In some embodiments, the nanoparticles have an average diameter of about 1000 nm for at least about 15 minutes after nanoparticle formation.

In some embodiments, the nanoparticles have an average diameter of about 1000 nm or less for at least about 4 hours after nanoparticle formation. In some embodiments, the nanoparticles have an average diameter of about 950 nm or less for at least about 4 hours after nanoparticle formation. In some embodiments, the nanoparticles have an average diameter of about 900 nm or less for at least about 4 hours after nanoparticle formation. In some embodiments, the nanoparticles have an average diameter of about 850 nm or less for at least about 4 hours after nanoparticle formation. In some embodiments, the nanoparticles have an average diameter of about 800 nm or less for at least about 4 hours after nanoparticle formation. In some embodiments, the nanoparticles have an average diameter of about 750 nm or less for at least about 4 hours after nanoparticle formation. In some embodiments, the nanoparticles have an average diameter of about 700 nm or less for at least about 4 hours after nanoparticle formation. In some embodiments, the nanoparticles have an average diameter of about 650 nm or less for at least about 4 hours after nanoparticle formation. In some embodiments, the nanoparticles have an average diameter of about 600 nm or less for at least about 4 hours after nanoparticle formation. In some embodiments, the nanoparticles have an average diameter of about 550 nm or less for at least about 4 hours after nanoparticle formation. In some embodiments, the nanoparticles have an average diameter of about 500 nm or less for at least about 4 hours after nanoparticle formation. In some embodiments, the nanoparticles have an average diameter of about 450 nm or less for at least about 4 hours after nanoparticle formation. In some embodiments, the nanoparticles have an average diameter of about 400 nm or less for at least about 4 hours after nanoparticle formation. In some embodiments, the nanoparticles have an average diameter of about 350 nm or less for at least about 4 hours after nanoparticle formation. In some embodiments, the nanoparticles have an average diameter of about 300 nm or less for at least about 4 hours after nanoparticle formation. In some embodiments, the nanoparticles have an average diameter of about 250 nm or less for at least about 4 hours after nanoparticle formation. In some embodiments, the nanoparticles have an average diameter of about 240 nm or less for at least about 4 hours after nanoparticle formation. In some embodiments, the nanoparticles have an average diameter of about 230 nm or less for at least about 4 hours after nanoparticle formation. In some embodiments, the nanoparticles have an average diameter of about 220 nm or less for at least about 4 hours after nanoparticle formation. In some embodiments, the nanoparticles have an average diameter of about 210 nm or less for at least about 4 hours after nanoparticle formation. In some embodiments, the nanoparticles have an average diameter of about 200 nm or less for at least about 4 hours after nanoparticle formation. In some embodiments, the nanoparticles have an average diameter of about 190 nm or less for at least about 4 hours after nanoparticle formation. In some embodiments, the nanoparticles have an average diameter of about 180 nm or less for at least about 4 hours after nanoparticle formation. In some embodiments, the nanoparticles have an average diameter of about 170 nm or less for at least about 4 hours after nanoparticle formation. In some embodiments, the nanoparticles have an average diameter of about 160 nm or less for at least about 4 hours after nanoparticle formation. In some embodiments, the nanoparticles have an average diameter of about 150 nm or less for at least about 4 hours after nanoparticle formation. In some embodiments, the nanoparticles have an average diameter of about 140 nm or less for at least about 4 hours after nanoparticle formation. In some embodiments, the nanoparticles have an average diameter of about 130 nm or less for at least about 4 hours after nanoparticle formation. In some embodiments, the nanoparticles have an average diameter of about 120 nm or less for at least about 4 hours after nanoparticle formation. In some embodiments, the nanoparticles have an average diameter of about 110 nm or less for at least about 4 hours after nanoparticle formation. In some embodiments, the nanoparticles have an average diameter of about 100 nm or less for at least about 4 hours after nanoparticle formation. In some embodiments, the nanoparticles have an average diameter of about 90 nm or less for at least about 4 hours after nanoparticle formation. In some embodiments, the nanoparticles have an average diameter of about 80 nm or less for at least about 4 hours after nanoparticle formation. In some embodiments, the nanoparticles have an average diameter of about 70 nm or less for at least about 4 hours after nanoparticle formation. In some embodiments, the nanoparticles have an average diameter of about 60 nm or less for at least about 4 hours after nanoparticle formation. In some embodiments, the nanoparticles have an average diameter of about 50 nm or less for at least about 4 hours after nanoparticle formation. In some embodiments, the nanoparticles have an average diameter of about 40 nm or less for at least about 4 hours after nanoparticle formation. In some embodiments, the nanoparticles have an average diameter of about 30 nm or less for at least about 4 hours after nanoparticle formation. In some embodiments, the nanoparticles have an average diameter of about 20 nm or less for at least about 4 hours after nanoparticle formation. In some embodiments, the nanoparticles have an average diameter of about 10 nm or less for at least about 4 hours after nanoparticle formation.

In some embodiments, the nanoparticles have an average diameter of about 10 nm or greater for at least about 4 hours after nanoparticle formation. In some embodiments, the nanoparticles have an average diameter of about 20 nm or greater for at least about 4 hours after nanoparticle formation. In some embodiments, the nanoparticles have an average diameter of about 30 nm or greater for at least about 4 hours after nanoparticle formation. In some embodiments, the nanoparticles have an average diameter of about 40 nm or greater for at least about 4 hours after nanoparticle formation. In some embodiments, the nanoparticles have an average diameter of about 50 nm or greater for at least about 4 hours after nanoparticle formation. In some embodiments, the nanoparticles have an average diameter of about 60 nm or greater for at least about 4 hours after nanoparticle formation. In some embodiments, the nanoparticles have an average diameter of about 70 nm or greater for at least about 4 hours after nanoparticle formation. In some embodiments, the nanoparticles have an average diameter of about 80 nm or greater for at least about 4 hours after nanoparticle formation. In some embodiments, the nanoparticles have an average diameter of about 90 nm or greater for at least about 4 hours after nanoparticle formation. In some embodiments, the nanoparticles have an average diameter of about 100 nm or greater for at least about 4 hours after nanoparticle formation. In some embodiments, the nanoparticles have an average diameter of about 110 nm or greater for at least about 4 hours after nanoparticle formation. In some embodiments, the nanoparticles have an average diameter of about 120 nm or greater for at least about 4 hours after nanoparticle formation. In some embodiments, the nanoparticles have an average diameter of about 130 nm or greater for at least about 4 hours after nanoparticle formation. In some embodiments, the nanoparticles have an average diameter of about 140 nm or greater for at least about 4 hours after nanoparticle formation. In some embodiments, the nanoparticles have an average diameter of about 150 nm or greater for at least about 4 hours after nanoparticle formation. In some embodiments, the nanoparticles have an average diameter of about 160 nm or greater for at least about 4 hours after nanoparticle formation. In some embodiments, the nanoparticles have an average diameter of about 170 nm or greater for at least about 4 hours after nanoparticle formation. In some embodiments, the nanoparticles have an average diameter of about 180 nm or greater for at least about 4 hours after nanoparticle formation. In some embodiments, the nanoparticles have an average diameter of about 190 nm or greater for at least about 4 hours after nanoparticle formation. In some embodiments, the nanoparticles have an average diameter of about 200 nm or greater for at least about 4 hours after nanoparticle formation. In some embodiments, the nanoparticles have an average diameter of about 210 nm or greater for at least about 4 hours after nanoparticle formation. In some embodiments, the nanoparticles have an average diameter of about 220 nm or greater for at least about 4 hours after nanoparticle formation. In some embodiments, the nanoparticles have an average diameter of about 230 nm or greater for at least about 4 hours after nanoparticle formation. In some embodiments, the nanoparticles have an average diameter of about 240 nm or greater for at least about 4 hours after nanoparticle formation. In some embodiments, the nanoparticles have an average diameter of about 250 nm or greater for at least about 4 hours after nanoparticle formation. In some embodiments, the nanoparticles have an average diameter of about 300 nm or greater for at least about 4 hours after nanoparticle formation. In some embodiments, the nanoparticles have an average diameter of about 350 nm or greater for at least about 4 hours after nanoparticle formation. In some embodiments, the nanoparticles have an average diameter of about 400 nm or greater for at least about 4 hours after nanoparticle formation. In some embodiments, the nanoparticles have an average diameter of about 450 nm or greater for at least about 4 hours after nanoparticle formation. In some embodiments, the nanoparticles have an average diameter of about 500 nm or greater for at least about 4 hours after nanoparticle formation. In some embodiments, the nanoparticles have an average diameter of about 550 nm or greater for at least about 4 hours after nanoparticle formation. In some embodiments, the nanoparticles have an average diameter of about 600 nm or greater for at least about 4 hours after nanoparticle formation. In some embodiments, the nanoparticles have an average diameter of about 650 nm or greater for at least about 4 hours after nanoparticle formation. In some embodiments, the nanoparticles have an average diameter of about 700 nm or greater for at least about 4 hours after nanoparticle formation. In some embodiments, the nanoparticles have an average diameter of about 750 nm or greater for at least about 4 hours after nanoparticle formation. In some embodiments, the nanoparticles have an average diameter of about 800 nm or greater for at least about 4 hours after nanoparticle formation. In some embodiments, the nanoparticles have an average diameter of about 850 nm or greater for at least about 4 hours after nanoparticle formation. In some embodiments, the nanoparticles have an average diameter of about 900 nm or greater for at least about 4 hours after nanoparticle formation. In some embodiments, the nanoparticles have an average diameter of about 950 nm or greater for at least about 4 hours after nanoparticle formation.

In some embodiments, the nanoparticles have an average diameter of from about 10 nm to about 1000 nm for at least about 4 hours after nanoparticle formation. In some embodiments, the nanoparticles have an average diameter of from about 10 nm to about 950 nm for at least about 4 hours after nanoparticle formation. In some embodiments, the nanoparticles have an average diameter of from about 10 nm to about 900 nm for at least about 4 hours after nanoparticle formation. In some embodiments, the nanoparticles have an average diameter of from about 10 nm to about 850 nm for at least about 4 hours after nanoparticle formation. In some embodiments, the nanoparticles have an average diameter of from about 10 nm to about 800 nm for at least about 4 hours after nanoparticle formation. In some embodiments, the nanoparticles have an average diameter of from about 10 nm to about 750 nm for at least about 4 hours after nanoparticle formation. In some embodiments, the nanoparticles have an average diameter of from about 10 nm to about 700 nm for at least about 4 hours after nanoparticle formation. In some embodiments, the nanoparticles have an average diameter of from about 10 nm to about 650 nm for at least about 4 hours after nanoparticle formation. In some embodiments, the nanoparticles have an average diameter of from about 10 nm to about 600 nm for at least about 4 hours after nanoparticle formation. In some embodiments, the nanoparticles have an average diameter of from about 10 nm to about 550 nm for at least about 4 hours after nanoparticle formation for at least about 4 hours after nanoparticle formation. In some embodiments, the nanoparticles have an average diameter of from about 10 nm to about 500 nm for at least about 4 hours after nanoparticle formation. In some embodiments, the nanoparticles have an average diameter of from about 10 nm to about 450 nm for at least about 4 hours after nanoparticle formation. In some embodiments, the nanoparticles have an average diameter of from about 10 nm to about 400 nm for at least about 4 hours after nanoparticle formation. In some embodiments, the nanoparticles have an average diameter of from about 10 nm to about 350 nm for at least about 4 hours after nanoparticle formation. In some embodiments, the nanoparticles have an average diameter of from about 10 nm to about 300 nm for at least about 4 hours after nanoparticle formation. In some embodiments, the nanoparticles have an average diameter of from about 10 nm to about 250 nm for at least about 4 hours after nanoparticle formation. In some embodiments, the nanoparticles have an average diameter of from about 10 nm to about 240 nm for at least about 4 hours after nanoparticle formation. In some embodiments, the nanoparticles have an average diameter of from about 10 nm to about 230 nm for at least about 4 hours after nanoparticle formation. In some embodiments, the nanoparticles have an average diameter of from about 10 nm to about 220 nm for at least about 4 hours after nanoparticle formation. In some embodiments, the nanoparticles have an average diameter of from about 10 nm to about 210 nm for at least about 4 hours after nanoparticle formation. In some embodiments, the nanoparticles have an average diameter of from about 10 nm to about 200 nm for at least about 4 hours after nanoparticle formation. In some embodiments, the nanoparticles have an average diameter of from about 10 nm to about 190 nm for at least about 4 hours after nanoparticle formation. In some embodiments, the nanoparticles have an average diameter of from about 10 nm to about 180 nm for at least about 4 hours after nanoparticle formation. In some embodiments, the nanoparticles have an average diameter of from about 10 nm to about 170 nm for at least about 4 hours after nanoparticle formation. In some embodiments, the nanoparticles have an average diameter of from about 10 nm to about 160 nm for at least about 4 hours after nanoparticle formation. In some embodiments, the nanoparticles have an average diameter of from about 10 nm to about 150 nm for at least about 4 hours after nanoparticle formation. In some embodiments, the nanoparticles have an average diameter of from about 10 nm to about 140 nm for at least about 4 hours after nanoparticle formation. In some embodiments, the nanoparticles have an average diameter of from about 10 nm to about 130 nm for at least about 4 hours after nanoparticle formation. In some embodiments, the nanoparticles have an average diameter of from about 10 nm to about 120 nm for at least about 4 hours after nanoparticle formation. In some embodiments, the nanoparticles have an average diameter of from about 10 nm to about 110 nm for at least about 4 hours after nanoparticle formation. In some embodiments, the nanoparticles have an average diameter of from about 10 nm to about 100 nm for at least about 4 hours after nanoparticle formation. In some embodiments, the nanoparticles have an average diameter of from about 10 nm to about 90 nm for at least about 4 hours after nanoparticle formation. In some embodiments, the nanoparticles have an average diameter of from about 10 nm to about 80 nm for at least about 4 hours after nanoparticle formation. In some embodiments, the nanoparticles have an average diameter of from about 10 nm to about 70 nm for at least about 4 hours after nanoparticle formation. In some embodiments, the nanoparticles have an average diameter of from about 10 nm to about 60 nm for at least about 4 hours after nanoparticle formation. In some embodiments, the nanoparticles have an average diameter of from about 10 nm to about 50 nm for at least about 4 hours after nanoparticle formation. In some embodiments, the nanoparticles have an average diameter of from about 10 nm to about 40 nm for at least about 4 hours after nanoparticle formation. In some embodiments, the nanoparticles have an average diameter of from about 10 nm to about 30 nm for at least about 4 hours after nanoparticle formation. In some embodiments, the nanoparticles have an average diameter of from about 10 nm to about 20 nm for at least about 4 hours after nanoparticle formation.

In some embodiments, the nanoparticles have an average diameter of about 10 nm for at least about 4 hours after nanoparticle formation. In some embodiments, the nanoparticles have an average diameter of about 20 nm for at least about 4 hours after nanoparticle formation. In some embodiments, the nanoparticles have an average diameter of about 30 nm for at least about 4 hours after nanoparticle formation. In some embodiments, the nanoparticles have an average diameter of about 40 nm for at least about 4 hours after nanoparticle formation. In some embodiments, the nanoparticles have an average diameter of about 50 nm for at least about 4 hours after nanoparticle formation. In some embodiments, the nanoparticles have an average diameter of about 60 nm for at least about 4 hours after nanoparticle formation. In some embodiments, the nanoparticles have an average diameter of about 70 nm for at least about 4 hours after nanoparticle formation. In some embodiments, the nanoparticles have an average diameter of about 80 nm for at least about 4 hours after nanoparticle formation. In some embodiments, the nanoparticles have an average diameter of about 90 nm for at least about 4 hours after nanoparticle formation. In some embodiments, the nanoparticles have an average diameter of about 100 nm for at least about 4 hours after nanoparticle formation. In some embodiments, the nanoparticles have an average diameter of about 110 nm for at least about 4 hours after nanoparticle formation. In some embodiments, the nanoparticles have an average diameter of about 120 nm for at least about 4 hours after nanoparticle formation. In some embodiments, the nanoparticles have an average diameter of about 130 nm for at least about 4 hours after nanoparticle formation. In some embodiments, the nanoparticles have an average diameter of about 140 nm for at least about 4 hours after nanoparticle formation. In some embodiments, the nanoparticles have an average diameter of about 150 nm for at least about 4 hours after nanoparticle formation. In some embodiments, the nanoparticles have an average diameter of about 160 nm for at least about 4 hours after nanoparticle formation. In some embodiments, the nanoparticles have an average diameter of about 170 nm for at least about 4 hours after nanoparticle formation. In some embodiments, the nanoparticles have an average diameter of about 180 nm for at least about 4 hours after nanoparticle formation. In some embodiments, the nanoparticles have an average diameter of about 190 nm for at least about 4 hours after nanoparticle formation. In some embodiments, the nanoparticles have an average diameter of about 200 nm for at least about 4 hours after nanoparticle formation. In some embodiments, the nanoparticles have an average diameter of about 210 nm for at least about 4 hours after nanoparticle formation. In some embodiments, the nanoparticles have an average diameter of about 220 nm for at least about 4 hours after nanoparticle formation. In some embodiments, the nanoparticles have an average diameter of about 230 nm for at least about 4 hours after nanoparticle formation. In some embodiments, the nanoparticles have an average diameter of about 240 nm for at least about 4 hours after nanoparticle formation. In some embodiments, the nanoparticles have an average diameter of about 250 nm for at least about 4 hours after nanoparticle formation. In some embodiments, the nanoparticles have an average diameter of about 300 nm for at least about 4 hours after nanoparticle formation. In some embodiments, the nanoparticles have an average diameter of about 350 nm for at least about 4 hours after nanoparticle formation. In some embodiments, the nanoparticles have an average diameter of about 400 nm for at least about 4 hours after nanoparticle formation. In some embodiments, the nanoparticles have an average diameter of about 450 nm for at least about 4 hours after nanoparticle formation. In some embodiments, the nanoparticles have an average diameter of about 500 nm for at least about 4 hours after nanoparticle formation. In some embodiments, the nanoparticles have an average diameter of about 550 nm for at least about 4 hours after nanoparticle formation. In some embodiments, the nanoparticles have an average diameter of about 600 nm for at least about 4 hours after nanoparticle formation. In some embodiments, the nanoparticles have an average diameter of about 650 nm for at least about 4 hours after nanoparticle formation. In some embodiments, the nanoparticles have an average diameter of about 700 nm for at least about 4 hours after nanoparticle formation. In some embodiments, the nanoparticles have an average diameter of about 750 nm for at least about 4 hours after nanoparticle formation. In some embodiments, the nanoparticles have an average diameter of about 800 nm for at least about 4 hours after nanoparticle formation. In some embodiments, the nanoparticles have an average diameter of about 850 nm for at least about 4 hours after nanoparticle formation. In some embodiments, the nanoparticles have an average diameter of about 900 nm for at least about 4 hours after nanoparticle formation. In some embodiments, the nanoparticles have an average diameter of about 950 nm for at least about 4 hours after nanoparticle formation. In some embodiments, the nanoparticles have an average diameter of about 1000 nm for at least about 4 hours after nanoparticle formation.

In some embodiments, the nanoparticles have an average diameter of from about 10 nm to about 1000 nm. In some embodiments, the nanoparticles have an average diameter of from about 10 nm to about 950 nm. In some embodiments, the nanoparticles have an average diameter of from about 10 nm to about 900 nm. In some embodiments, the nanoparticles have an average diameter of from about 10 nm to about 850 nm. In some embodiments, the nanoparticles have an average diameter of from about 10 nm to about 800 nm. In some embodiments, the nanoparticles have an average diameter of from about 10 nm to about 750 nm. In some embodiments, the nanoparticles have an average diameter of from about 10 nm to about 700 nm. In some embodiments, the nanoparticles have an average diameter of from about 10 nm to about 650 nm. In some embodiments, the nanoparticles have an average diameter of from about 10 nm to about 600 nm. In some embodiments, the nanoparticles have an average diameter of from about 10 nm to about 550 nm. In some embodiments, the nanoparticles have an average diameter of from about 10 nm to about 500 nm. In some embodiments, the nanoparticles have an average diameter of from about 10 nm to about 450 nm. In some embodiments, the nanoparticles have an average diameter of from about 10 nm to about 400 nm. In some embodiments, the nanoparticles have an average diameter of from about 10 nm to about 350 nm. In some embodiments, the nanoparticles have an average diameter of from about 10 nm to about 300 nm. In some embodiments, the nanoparticles have an average diameter of from about 10 nm to about 250 nm. In some embodiments, the nanoparticles have an average diameter of from about 10 nm to about 240 nm. In some embodiments, the nanoparticles have an average diameter of from about 10 nm to about 230 nm. In some embodiments, the nanoparticles have an average diameter of from about 10 nm to about 220 nm. In some embodiments, the nanoparticles have an average diameter of from about 10 nm to about 210 nm. In some embodiments, the nanoparticles have an average diameter of from about 10 nm to about 200 nm. In some embodiments, the nanoparticles have an average diameter of from about 10 nm to about 190 nm. In some embodiments, the nanoparticles have an average diameter of from about 10 nm to about 180 nm. In some embodiments, the nanoparticles have an average diameter of from about 10 nm to about 170 nm. In some embodiments, the nanoparticles have an average diameter of from about 10 nm to about 160 nm. In some embodiments, the nanoparticles have an average diameter of from about 10 nm to about 150 nm. In some embodiments, the nanoparticles have an average diameter of from about 10 nm to about 140 nm. In some embodiments, the nanoparticles have an average diameter of from about 10 nm to about 130 nm. In some embodiments, the nanoparticles have an average diameter of from about 10 nm to about 120 nm. In some embodiments, the nanoparticles have an average diameter of from about 10 nm to about 110 nm. In some embodiments, the nanoparticles have an average diameter of from about 10 nm to about 100 nm. In some embodiments, the nanoparticles have an average diameter of from about 10 nm to about 90 nm. In some embodiments, the nanoparticles have an average diameter of from about 10 nm to about 80 nm. In some embodiments, the nanoparticles have an average diameter of from about 10 nm to about 70 nm. In some embodiments, the nanoparticles have an average diameter of from about 10 nm to about 60 nm. In some embodiments, the nanoparticles have an average diameter of from about 10 nm to about 50 nm. In some embodiments, the nanoparticles have an average diameter of from about 10 nm to about 40 nm. In some embodiments, the nanoparticles have an average diameter of from about 10 nm to about 30 nm. In some embodiments, the nanoparticles have an average diameter of from about 10 nm to about 20 nm.

In some embodiments, the nanoparticles have an average diameter of from about 20 nm to about 1000 nm. In some embodiments, the nanoparticles have an average diameter of from about 20 nm to about 950 nm. In some embodiments, the nanoparticles have an average diameter of from about 20 nm to about 900 nm. In some embodiments, the nanoparticles have an average diameter of from about 20 nm to about 850 nm. In some embodiments, the nanoparticles have an average diameter of from about 20 nm to about 800 nm. In some embodiments, the nanoparticles have an average diameter of from about 20 nm to about 750 nm. In some embodiments, the nanoparticles have an average diameter of from about 20 nm to about 700 nm. In some embodiments, the nanoparticles have an average diameter of from about 20 nm to about 650 nm. In some embodiments, the nanoparticles have an average diameter of from about 20 nm to about 600 nm. In some embodiments, the nanoparticles have an average diameter of from about 20 nm to about 550 nm. In some embodiments, the nanoparticles have an average diameter of from about 20 nm to about 500 nm. In some embodiments, the nanoparticles have an average diameter of from about 20 nm to about 450 nm. In some embodiments, the nanoparticles have an average diameter of from about 20 nm to about 400 nm. In some embodiments, the nanoparticles have an average diameter of from about 20 nm to about 350 nm. In some embodiments, the nanoparticles have an average diameter of from about 20 nm to about 300 nm. In some embodiments, the nanoparticles have an average diameter of from about 20 nm to about 250 nm. In some embodiments, the nanoparticles have an average diameter of from about 20 nm to about 240 nm. In some embodiments, the nanoparticles have an average diameter of from about 20 nm to about 230 nm. In some embodiments, the nanoparticles have an average diameter of from about 20 nm to about 220 nm. In some embodiments, the nanoparticles have an average diameter of from about 20 nm to about 210 nm. In some embodiments, the nanoparticles have an average diameter of from about 20 nm to about 200 nm. In some embodiments, the nanoparticles have an average diameter of from about 20 nm to about 190 nm. In some embodiments, the nanoparticles have an average diameter of from about 20 nm to about 180 nm. In some embodiments, the nanoparticles have an average diameter of from about 20 nm to about 170 nm. In some embodiments, the nanoparticles have an average diameter of from about 20 nm to about 160 nm. In some embodiments, the nanoparticles have an average diameter of from about 20 nm to about 150 nm. In some embodiments, the nanoparticles have an average diameter of from about 20 nm to about 140 nm. In some embodiments, the nanoparticles have an average diameter of from about 20 nm to about 130 nm. In some embodiments, the nanoparticles have an average diameter of from about 20 nm to about 120 nm. In some embodiments, the nanoparticles have an average diameter of from about 20 nm to about 110 nm. In some embodiments, the nanoparticles have an average diameter of from about 20 nm to about 100 nm. In some embodiments, the nanoparticles have an average diameter of from about 20 nm to about 90 nm. In some embodiments, the nanoparticles have an average diameter of from about 20 nm to about 80 nm. In some embodiments, the nanoparticles have an average diameter of from about 20 nm to about 70 nm. In some embodiments, the nanoparticles have an average diameter of from about 20 nm to about 60 nm. In some embodiments, the nanoparticles have an average diameter of from about 20 nm to about 50 nm. In some embodiments, the nanoparticles have an average diameter of from about 20 nm to about 40 nm. In some embodiments, the nanoparticles have an average diameter of from about 20 nm to about 30 nm.

In some embodiments, the nanoparticles have an average diameter of from about 30 nm to about 1000 nm. In some embodiments, the nanoparticles have an average diameter of from about 30 nm to about 950 nm. In some embodiments, the nanoparticles have an average diameter of from about 30 nm to about 900 nm. In some embodiments, the nanoparticles have an average diameter of from about 30 nm to about 850 nm. In some embodiments, the nanoparticles have an average diameter of from about 30 nm to about 800 nm. In some embodiments, the nanoparticles have an average diameter of from about 30 nm to about 750 nm. In some embodiments, the nanoparticles have an average diameter of from about 30 nm to about 700 nm. In some embodiments, the nanoparticles have an average diameter of from about 30 nm to about 650 nm. In some embodiments, the nanoparticles have an average diameter of from about 30 nm to about 600 nm. In some embodiments, the nanoparticles have an average diameter of from about 30 nm to about 550 nm. In some embodiments, the nanoparticles have an average diameter of from about 30 nm to about 500 nm. In some embodiments, the nanoparticles have an average diameter of from about 30 nm to about 450 nm. In some embodiments, the nanoparticles have an average diameter of from about 30 nm to about 400 nm. In some embodiments, the nanoparticles have an average diameter of from about 30 nm to about 350 nm. In some embodiments, the nanoparticles have an average diameter of from about 30 nm to about 300 nm. In some embodiments, the nanoparticles have an average diameter of from about 30 nm to about 250 nm. In some embodiments, the nanoparticles have an average diameter of from about 30 nm to about 240 nm. In some embodiments, the nanoparticles have an average diameter of from about 30 nm to about 230 nm. In some embodiments, the nanoparticles have an average diameter of from about 30 nm to about 220 nm. In some embodiments, the nanoparticles have an average diameter of from about 30 nm to about 210 nm. In some embodiments, the nanoparticles have an average diameter of from about 30 nm to about 200 nm. In some embodiments, the nanoparticles have an average diameter of from about 30 nm to about 190 nm. In some embodiments, the nanoparticles have an average diameter of from about 30 nm to about 180 nm. In some embodiments, the nanoparticles have an average diameter of from about 30 nm to about 170 nm. In some embodiments, the nanoparticles have an average diameter of from about 30 nm to about 160 nm. In some embodiments, the nanoparticles have an average diameter of from about 30 nm to about 150 nm. In some embodiments, the nanoparticles have an average diameter of from about 30 nm to about 140 nm. In some embodiments, the nanoparticles have an average diameter of from about 30 nm to about 130 nm. In some embodiments, the nanoparticles have an average diameter of from about 30 nm to about 120 nm. In some embodiments, the nanoparticles have an average diameter of from about 30 nm to about 110 nm. In some embodiments, the nanoparticles have an average diameter of from about 30 nm to about 100 nm. In some embodiments, the nanoparticles have an average diameter of from about 30 nm to about 90 nm. In some embodiments, the nanoparticles have an average diameter of from about 30 nm to about 80 nm. In some embodiments, the nanoparticles have an average diameter of from about 30 nm to about 70 nm. In some embodiments, the nanoparticles have an average diameter of from about 30 nm to about 60 nm. In some embodiments, the nanoparticles have an average diameter of from about 30 nm to about 50 nm. In some embodiments, the nanoparticles have an average diameter of from about 30 nm to about 40 nm. In some embodiments, the nanoparticles have an average diameter of from about 30 nm to about 40 nm.

In some embodiments, the nanoparticles have an average diameter of from about 40 nm to about 1000 nm. In some embodiments, the nanoparticles have an average diameter of from about 40 nm to about 950 nm. In some embodiments, the nanoparticles have an average diameter of from about 40 nm to about 900 nm. In some embodiments, the nanoparticles have an average diameter of from about 40 nm to about 850 nm. In some embodiments, the nanoparticles have an average diameter of from about 40 nm to about 800 nm. In some embodiments, the nanoparticles have an average diameter of from about 40 nm to about 750 nm. In some embodiments, the nanoparticles have an average diameter of from about 40 nm to about 700 nm. In some embodiments, the nanoparticles have an average diameter of from about 40 nm to about 650 nm. In some embodiments, the nanoparticles have an average diameter of from about 40 nm to about 600 nm. In some embodiments, the nanoparticles have an average diameter of from about 40 nm to about 550 nm. In some embodiments, the nanoparticles have an average diameter of from about 40 nm to about 500 nm. In some embodiments, the nanoparticles have an average diameter of from about 40 nm to about 450 nm. In some embodiments, the nanoparticles have an average diameter of from about 40 nm to about 400 nm. In some embodiments, the nanoparticles have an average diameter of from about 40 nm to about 350 nm. In some embodiments, the nanoparticles have an average diameter of from about 40 nm to about 300 nm. In some embodiments, the nanoparticles have an average diameter of from about 40 nm to about 250 nm. In some embodiments, the nanoparticles have an average diameter of from about 40 nm to about 240 nm. In some embodiments, the nanoparticles have an average diameter of from about 40 nm to about 230 nm. In some embodiments, the nanoparticles have an average diameter of from about 40 nm to about 220 nm. In some embodiments, the nanoparticles have an average diameter of from about 40 nm to about 210 nm. In some embodiments, the nanoparticles have an average diameter of from about 40 nm to about 200 nm. In some embodiments, the nanoparticles have an average diameter of from about 40 nm to about 190 nm. In some embodiments, the nanoparticles have an average diameter of from about 40 nm to about 180 nm. In some embodiments, the nanoparticles have an average diameter of from about 40 nm to about 170 nm. In some embodiments, the nanoparticles have an average diameter of from about 40 nm to about 160 nm. In some embodiments, the nanoparticles have an average diameter of from about 40 nm to about 150 nm. In some embodiments, the nanoparticles have an average diameter of from about 40 nm to about 140 nm. In some embodiments, the nanoparticles have an average diameter of from about 40 nm to about 130 nm. In some embodiments, the nanoparticles have an average diameter of from about 40 nm to about 120 nm. In some embodiments, the nanoparticles have an average diameter of from about 40 nm to about 110 nm. In some embodiments, the nanoparticles have an average diameter of from about 40 nm to about 100 nm. In some embodiments, the nanoparticles have an average diameter of from about 40 nm to about 90 nm. In some embodiments, the nanoparticles have an average diameter of from about 40 nm to about 80 nm. In some embodiments, the nanoparticles have an average diameter of from about 40 nm to about 70 nm. In some embodiments, the nanoparticles have an average diameter of from about 40 nm to about 60 nm. In some embodiments, the nanoparticles have an average diameter of from about 40 nm to about 50 nm.

In some embodiments, the nanoparticles have an average diameter of from about 50 nm to about 1000 nm. In some embodiments, the nanoparticles have an average diameter of from about 50 nm to about 950 nm. In some embodiments, the nanoparticles have an average diameter of from about 50 nm to about 900 nm. In some embodiments, the nanoparticles have an average diameter of from about 50 nm to about 850 nm. In some embodiments, the nanoparticles have an average diameter of from about 50 nm to about 800 nm. In some embodiments, the nanoparticles have an average diameter of from about 50 nm to about 750 nm. In some embodiments, the nanoparticles have an average diameter of from about 50 nm to about 700 nm. In some embodiments, the nanoparticles have an average diameter of from about 50 nm to about 650 nm. In some embodiments, the nanoparticles have an average diameter of from about 50 nm to about 600 nm. In some embodiments, the nanoparticles have an average diameter of from about 50 nm to about 550 nm. In some embodiments, the nanoparticles have an average diameter of from about 50 nm to about 500 nm. In some embodiments, the nanoparticles have an average diameter of from about 50 nm to about 450 nm. In some embodiments, the nanoparticles have an average diameter of from about 50 nm to about 400 nm. In some embodiments, the nanoparticles have an average diameter of from about 50 nm to about 350 nm. In some embodiments, the nanoparticles have an average diameter of from about 50 nm to about 300 nm. In some embodiments, the nanoparticles have an average diameter of from about 50 nm to about 250 nm. In some embodiments, the nanoparticles have an average diameter of from about 50 nm to about 240 nm. In some embodiments, the nanoparticles have an average diameter of from about 50 nm to about 230 nm. In some embodiments, the nanoparticles have an average diameter of from about 50 nm to about 220 nm. In some embodiments, the nanoparticles have an average diameter of from about 50 nm to about 210 nm. In some embodiments, the nanoparticles have an average diameter of from about 50 nm to about 200 nm. In some embodiments, the nanoparticles have an average diameter of from about 50 nm to about 190 nm. In some embodiments, the nanoparticles have an average diameter of from about 50 nm to about 180 nm. In some embodiments, the nanoparticles have an average diameter of from about 50 nm to about 170 nm. In some embodiments, the nanoparticles have an average diameter of from about 50 nm to about 160 nm. In some embodiments, the nanoparticles have an average diameter of from about 50 nm to about 150 nm. In some embodiments, the nanoparticles have an average diameter of from about 50 nm to about 140 nm. In some embodiments, the nanoparticles have an average diameter of from about 50 nm to about 130 nm. In some embodiments, the nanoparticles have an average diameter of from about 50 nm to about 120 nm. In some embodiments, the nanoparticles have an average diameter of from about 50 nm to about 110 nm. In some embodiments, the nanoparticles have an average diameter of from about 50 nm to about 100 nm. In some embodiments, the nanoparticles have an average diameter of from about 50 nm to about 90 nm. In some embodiments, the nanoparticles have an average diameter of from about 50 nm to about 80 nm. In some embodiments, the nanoparticles have an average diameter of from about 50 nm to about 70 nm. In some embodiments, the nanoparticles have an average diameter of from about 50 nm to about 60 nm.

In some embodiments, the nanoparticles have an average diameter of about 10 nm. In some embodiments, the nanoparticles have an average diameter of about 20 nm. In some embodiments, the nanoparticles have an average diameter of about 30 nm. In some embodiments, the nanoparticles have an average diameter of about 40 nm. In some embodiments, the nanoparticles have an average diameter of about 50 nm. In some embodiments, the nanoparticles have an average diameter of about 60 nm. In some embodiments, the nanoparticles have an average diameter of about 70 nm. In some embodiments, the nanoparticles have an average diameter of about 80 nm. In some embodiments, the nanoparticles have an average diameter of about 90 nm. In some embodiments, the nanoparticles have an average diameter of about 100 nm. In some embodiments, the nanoparticles have an average diameter of about 110 nm. In some embodiments, the nanoparticles have an average diameter of about 120 nm. In some embodiments, the nanoparticles have an average diameter of about 130 nm. In some embodiments, the nanoparticles have an average diameter of about 140 nm. In some embodiments, the nanoparticles have an average diameter of about 150 nm. In some embodiments, the nanoparticles have an average diameter of about 160 nm. In some embodiments, the nanoparticles have an average diameter of about 170 nm. In some embodiments, the nanoparticles have an average diameter of about 180 nm. In some embodiments, the nanoparticles have an average diameter of about 190 nm. In some embodiments, the nanoparticles have an average diameter of about 200 nm. In some embodiments, the nanoparticles have an average diameter of about 210 nm. In some embodiments, the nanoparticles have an average diameter of about 220 nm. In some embodiments, the nanoparticles have an average diameter of about 230 nm. In some embodiments, the nanoparticles have an average diameter of about 240 nm. In some embodiments, the nanoparticles have an average diameter of about 250 nm. In some embodiments, the nanoparticles have an average diameter of about 300 nm. In some embodiments, the nanoparticles have an average diameter of about 350 nm. In some embodiments, the nanoparticles have an average diameter of about 400 nm. In some embodiments, the nanoparticles have an average diameter of about 450 nm. In some embodiments, the nanoparticles have an average diameter of about 500 nm. In some embodiments, the nanoparticles have an average diameter of about 550 nm. In some embodiments, the nanoparticles have an average diameter of about 600 nm. In some embodiments, the nanoparticles have an average diameter of about 650 nm. In some embodiments, the nanoparticles have an average diameter of about 700 nm. In some embodiments, the nanoparticles have an average diameter of about 750 nm. In some embodiments, the nanoparticles have an average diameter of about 800 nm. In some embodiments, the nanoparticles have an average diameter of about 850 nm. In some embodiments, the nanoparticles have an average diameter of about 900 nm. In some embodiments, the nanoparticles have an average diameter of about 950 nm. In some embodiments, the nanoparticles have an average diameter of about 1000 nm.

In some embodiments, the composition is sterile filterable. In some embodiments, the nanoparticles have an average diameter of about 250 nm or less. In some embodiments, the nanoparticles have an average diameter of about 240 nm or less. In some embodiments, the nanoparticles have an average diameter of about 230 nm or less. In some embodiments, the nanoparticles have an average diameter of about 220 nm or less. In some embodiments, the nanoparticles have an average diameter of about 210 nm or less. In some embodiments, the nanoparticles have an average diameter of about 200 nm or less. In some embodiments, the nanoparticles have an average diameter of from about 10 nm to about 250 nm. In some embodiments, the nanoparticles have an average diameter of from about 10 nm to about 240 nm. In some embodiments, the nanoparticles have an average diameter of from about 10 nm to about 230 nm. In some embodiments, the nanoparticles have an average diameter of from about 10 nm to about 220 nm. In some embodiments, the nanoparticles have an average diameter of from about 10 nm to about 210 nm. In some embodiments, the nanoparticles have an average diameter of from about 10 nm to about 200 nm.

In some embodiments, the nanoparticles are suspended, dissolved, or emulsified in a liquid. In some embodiments, the nanoparticles are suspended in a liquid. In some embodiments, the nanoparticles are dissolved in a liquid. In some embodiments, the nanoparticles are emulsified in a liquid.

### Dehydrated Composition

In some embodiments, the composition is dehydrated. In some embodiments, the composition is a lyophilized composition. In some embodiments, the dehydrated composition comprises less than about 10%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.05%, or about 0.01% by weight of water. In some embodiments, the dehydrated composition comprises less than about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.05%, or about 0.01% by weight of water.

In some embodiments, when the composition is dehydrated composition, such as a lyophilized composition, the composition comprises from about 0.1% to about 99% by weight of the prodrug of the nucleoside or nucleotide. In some embodiments, the composition comprises from about 0.1% to about 75% by weight of the prodrug of the nucleoside or nucleotide. In some embodiments, the composition comprises from about 0.1% to about 50% by weight of the prodrug of the nucleoside or nucleotide. In some embodiments, the composition comprises from about 0.1% to about 25% by weight of the prodrug of the nucleoside or nucleotide. In some embodiments, the composition comprises from about 0.1% to about 20% by weight of the prodrug of the nucleoside or nucleotide. In some embodiments, the composition comprises from about 0.1% to about 15% by weight of the prodrug of the nucleoside or nucleotide. In some embodiments, the composition comprises from about 0.1% to about 10% by weight of the prodrug of the nucleoside or nucleotide.

In some embodiments, when the composition is dehydrated composition, such as a lyophilized composition, the composition comprises from about 0.5% to about 99% by weight of the prodrug of the nucleoside or nucleotide. In some embodiments, the composition comprises from about 0.5% to about 75% by weight of the prodrug of the nucleoside or nucleotide. In some embodiments, the composition comprises from about 0.5% to about 50% by weight of the prodrug of the nucleoside or nucleotide. In some embodiments, the composition comprises from about 0.5% to about 25% by weight of the prodrug of the nucleoside or nucleotide. In some embodiments, the composition comprises from about 0.5% to about 20% by weight of the prodrug of the nucleoside or nucleotide. In some embodiments, the composition comprises from about 0.5% to about 15% by weight of the prodrug of the nucleoside or nucleotide. In some embodiments, the composition comprises from about 0.5% to about 10% by weight of the prodrug of the nucleoside or nucleotide.

In some embodiments, when the composition is dehydrated composition, such as a lyophilized composition, the composition comprises from about 0.9% to about 24% by weight of the prodrug of the nucleoside or nucleotide. In some embodiments, the composition comprises from about 1.8% to about 16% by weight of the prodrug of the nucleoside or nucleotide.

In some embodiments, when the composition is dehydrated composition, such as a lyophilized composition, the composition comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9% about ^{2%}, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% by weight of the prodrug of the nucleoside or nucleotide. In some embodiments, the composition comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the prodrug of the nucleoside or nucleotide. In some embodiments, the composition comprises about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, or about 24% by weight of the prodrug of the nucleoside or nucleotide. In some embodiments, the composition comprises about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, or about 16% by weight of the prodrug of the nucleoside or nucleotide.

In some embodiments, when the composition is dehydrated composition, such as a lyophilized composition, the composition comprises from about 50% to about 99% by weight of the pharmaceutically acceptable carrier. In some embodiments, the composition comprises from about 55% to about 99% by weight of the pharmaceutically acceptable carrier. In some embodiments, the composition comprises from about 60% to about 99% by weight of the pharmaceutically acceptable carrier. In some embodiments, the composition comprises from about 65% to about 99% by weight of the pharmaceutically acceptable carrier. In some embodiments, the composition comprises from about 70% to about 99% by weight of the pharmaceutically acceptable carrier. In some embodiments, the composition comprises from about 75% to about 99% by weight of the pharmaceutically acceptable carrier. In some embodiments, the composition comprises from about 80% to about 99% by weight of the pharmaceutically acceptable carrier. In some embodiments, the composition comprises from about 85% to about 99% by weight of the pharmaceutically acceptable carrier. In some embodiments, the composition comprises from about 90% to about 99% by weight of the pharmaceutically acceptable carrier.

In some embodiments, when the composition is dehydrated composition, such as a lyophilized composition, the composition comprises from about 76% to about 99% by weight of the pharmaceutically acceptable carrier. In some embodiments, the composition comprises from about 84% to about 98% by weight of the pharmaceutically acceptable carrier.

In some embodiments, when the composition is dehydrated composition, such as a lyophilized composition, the composition comprises about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier. In some embodiments, the composition comprises about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier. In some embodiments, the composition comprises about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier.

### Reconstitution

In some embodiments, the composition is reconstituted with an appropriate biocompatible liquid to provide a reconstituted composition. In some embodiments, appropriate biocompatible liquid is a buffered solution. Examples of suitable buffered solutions include, but are not limited to, buffered solutions of amino acids, buffered solutions of proteins, buffered solutions of sugars, buffered solutions of vitamins, buffered solutions of synthetic polymers, buffered solutions of salts (such as buffered saline or buffered aqueous media), any similar buffered solutions, or any suitable combination thereof. In some embodiments, the appropriate biocompatible liquid is a solution comprising dextrose. In some embodiments, the appropriate biocompatible liquid is a solution comprising one or more salts. In some embodiments, the appropriate biocompatible liquid is a solution suitable for intravenous use. Examples of solutions that are suitable for intravenous use, include, but are not limited to, balanced solutions, which are different solutions with different electrolyte compositions that are close to plasma composition. Such electrolyte compositions comprise crystalloids or colloids. Examples of suitable appropriate biocompatible liquids include, but are not limited to, sterile water, saline, phosphate-buffered saline, 5% dextrose in water solution, Ringer's solution, or Ringer's lactate solution. In some embodiments, the appropriate biocompatible liquid is sterile water, saline, phosphate-buffered saline, 5% dextrose in water solution, Ringer's solution, or Ringer's lactate solution. In some embodiments, the appropriate biocompatible liquid is sterile water. In some embodiments, the appropriate biocompatible liquid is saline. In some embodiments, the appropriate biocompatible liquid is phosphate-buffered saline. In some embodiments, the appropriate biocompatible liquid is 5% dextrose in water solution. In some embodiments, the appropriate biocompatible liquid is Ringer's solution. In some embodiments, the appropriate biocompatible liquid is Ringer's lactate solution. In some embodiments, the appropriate biocompatible liquid is a balanced solution, or a solution with an electrolyte composition that resembles plasma.

In some embodiments, the nanoparticles have an average diameter of from about 10 nm to about 1000 nm after reconstitution. In some embodiments, the nanoparticles have an average diameter of from about 10 nm to about 950 nm after reconstitution. In some embodiments, the nanoparticles have an average diameter of from about 10 nm to about 900 nm after reconstitution. In some embodiments, the nanoparticles have an average diameter of from about 10 nm to about 850 nm after reconstitution. In some embodiments, the nanoparticles have an average diameter of from about 10 nm to about 800 nm after reconstitution. In some embodiments, the nanoparticles have an average diameter of from about 10 nm to about 750 nm after reconstitution. In some embodiments, the nanoparticles have an average diameter of from about 10 nm to about 700 nm after reconstitution. In some embodiments, the nanoparticles have an average diameter of from about 10 nm to about 650 nm after reconstitution. In some embodiments, the nanoparticles have an average diameter of from about 10 nm to about 600 nm after reconstitution. In some embodiments, the nanoparticles have an average diameter of from about 10 nm to about 550 nm after reconstitution. In some embodiments, the nanoparticles have an average diameter of from about 10 nm to about 500 nm after reconstitution. In some embodiments, the nanoparticles have an average diameter of from about 10 nm to about 450 nm after reconstitution. In some embodiments, the nanoparticles have an average diameter of from about 10 nm to about 400 nm after reconstitution. In some embodiments, the nanoparticles have an average diameter of from about 10 nm to about 350 nm after reconstitution. In some embodiments, the nanoparticles have an average diameter of from about 10 nm to about 300 nm after reconstitution. In some embodiments, the nanoparticles have an average diameter of from about 10 nm to about 250 nm after reconstitution. In some embodiments, the nanoparticles have an average diameter of from about 10 nm to about 240 nm after reconstitution. In some embodiments, the nanoparticles have an average diameter of from about 10 nm to about 230 nm after reconstitution. In some embodiments, the nanoparticles have an average diameter of from about 10 nm to about 220 nm after reconstitution. In some embodiments, the nanoparticles have an average diameter of from about 10 nm to about 210 nm after reconstitution. In some embodiments, the nanoparticles have an average diameter of from about 10 nm to about 200 nm after reconstitution. In some embodiments, the nanoparticles have an average diameter of from about 10 nm to about 190 nm after reconstitution. In some embodiments, the nanoparticles have an average diameter of from about 10 nm to about 180 nm after reconstitution. In some embodiments, the nanoparticles have an average diameter of from about 10 nm to about 170 nm after reconstitution. In some embodiments, the nanoparticles have an average diameter of from about 10 nm to about 160 nm after reconstitution. In some embodiments, the nanoparticles have an average diameter of from about 10 nm to about 150 nm after reconstitution. In some embodiments, the nanoparticles have an average diameter of from about 10 nm to about 140 nm after reconstitution. In some embodiments, the nanoparticles have an average diameter of from about 10 nm to about 130 nm after reconstitution. In some embodiments, the nanoparticles have an average diameter of from about 10 nm to about 120 nm after reconstitution. In some embodiments, the nanoparticles have an average diameter of from about 10 nm to about 110 nm after reconstitution. In some embodiments, the nanoparticles have an average diameter of from about 10 nm to about 100 nm after reconstitution. In some embodiments, the nanoparticles have an average diameter of from about 10 nm to about 90 nm after reconstitution. In some embodiments, the nanoparticles have an average diameter of from about 10 nm to about 80 nm after reconstitution. In some embodiments, the nanoparticles have an average diameter of from about 10 nm to about 70 nm after reconstitution. In some embodiments, the nanoparticles have an average diameter of from about 10 nm to about 60 nm after reconstitution. In some embodiments, the nanoparticles have an average diameter of from about 10 nm to about 50 nm after reconstitution. In some embodiments, the nanoparticles have an average diameter of from about 10 nm to about 40 nm after reconstitution. In some embodiments, the nanoparticles have an average diameter of from about 10 nm to about 30 nm after reconstitution. In some embodiments, the nanoparticles have an average diameter of from about 10 nm to about 20 nm after reconstitution.

In some embodiments, the nanoparticles have an average diameter of from about 20 nm to about 1000 nm after reconstitution. In some embodiments, the nanoparticles have an average diameter of from about 20 nm to about 950 nm after reconstitution. In some embodiments, the nanoparticles have an average diameter of from about 20 nm to about 900 nm after reconstitution. In some embodiments, the nanoparticles have an average diameter of from about 20 nm to about 850 nm after reconstitution. In some embodiments, the nanoparticles have an average diameter of from about 20 nm to about 800 nm after reconstitution. In some embodiments, the nanoparticles have an average diameter of from about 20 nm to about 750 nm after reconstitution. In some embodiments, the nanoparticles have an average diameter of from about 20 nm to about 700 nm after reconstitution. In some embodiments, the nanoparticles have an average diameter of from about 20 nm to about 650 nm after reconstitution. In some embodiments, the nanoparticles have an average diameter of from about 20 nm to about 600 nm after reconstitution. In some embodiments, the nanoparticles have an average diameter of from about 20 nm to about 550 nm after reconstitution. In some embodiments, the nanoparticles have an average diameter of from about 20 nm to about 500 nm after reconstitution. In some embodiments, the nanoparticles have an average diameter of from about 20 nm to about 450 nm after reconstitution. In some embodiments, the nanoparticles have an average diameter of from about 20 nm to about 400 nm after reconstitution. In some embodiments, the nanoparticles have an average diameter of from about 20 nm to about 350 nm after reconstitution. In some embodiments, the nanoparticles have an average diameter of from about 20 nm to about 300 nm after reconstitution. In some embodiments, the nanoparticles have an average diameter of from about 20 nm to about 250 nm after reconstitution. In some embodiments, the nanoparticles have an average diameter of from about 20 nm to about 240 nm after reconstitution. In some embodiments, the nanoparticles have an average diameter of from about 20 nm to about 230 nm after reconstitution. In some embodiments, the nanoparticles have an average diameter of from about 20 nm to about 220 nm after reconstitution. In some embodiments, the nanoparticles have an average diameter of from about 20 nm to about 210 nm after reconstitution. In some embodiments, the nanoparticles have an average diameter of from about 20 nm to about 200 nm after reconstitution. In some embodiments, the nanoparticles have an average diameter of from about 20 nm to about 190 nm after reconstitution. In some embodiments, the nanoparticles have an average diameter of from about 20 nm to about 180 nm after reconstitution. In some embodiments, the nanoparticles have an average diameter of from about 20 nm to about 170 nm after reconstitution. In some embodiments, the nanoparticles have an average diameter of from about 20 nm to about 160 nm after reconstitution. In some embodiments, the nanoparticles have an average diameter of from about 20 nm to about 150 nm after reconstitution. In some embodiments, the nanoparticles have an average diameter of from about 20 nm to about 140 nm after reconstitution. In some embodiments, the nanoparticles have an average diameter of from about 20 nm to about 130 nm after reconstitution. In some embodiments, the nanoparticles have an average diameter of from about 20 nm to about 120 nm after reconstitution. In some embodiments, the nanoparticles have an average diameter of from about 20 nm to about 110 nm after reconstitution. In some embodiments, the nanoparticles have an average diameter of from about 20 nm to about 100 nm after reconstitution. In some embodiments, the nanoparticles have an average diameter of from about 20 nm to about 90 nm after reconstitution. In some embodiments, the nanoparticles have an average diameter of from about 20 nm to about 80 nm after reconstitution. In some embodiments, the nanoparticles have an average diameter of from about 20 nm to about 70 nm after reconstitution. In some embodiments, the nanoparticles have an average diameter of from about 20 nm to about 60 nm after reconstitution. In some embodiments, the nanoparticles have an average diameter of from about 20 nm to about 50 nm after reconstitution. In some embodiments, the nanoparticles have an average diameter of from about 20 nm to about 40 nm after reconstitution. In some embodiments, the nanoparticles have an average diameter of from about 20 nm to about 30 nm after reconstitution.

In some embodiments, the nanoparticles have an average diameter of from about 30 nm to about 1000 nm after reconstitution. In some embodiments, the nanoparticles have an average diameter of from about 30 nm to about 950 nm after reconstitution. In some embodiments, the nanoparticles have an average diameter of from about 30 nm to about 900 nm after reconstitution. In some embodiments, the nanoparticles have an average diameter of from about 30 nm to about 850 nm after reconstitution. In some embodiments, the nanoparticles have an average diameter of from about 30 nm to about 800 nm after reconstitution. In some embodiments, the nanoparticles have an average diameter of from about 30 nm to about 750 nm after reconstitution. In some embodiments, the nanoparticles have an average diameter of from about 30 nm to about 700 nm after reconstitution. In some embodiments, the nanoparticles have an average diameter of from about 30 nm to about 650 nm after reconstitution. In some embodiments, the nanoparticles have an average diameter of from about 30 nm to about 600 nm after reconstitution. In some embodiments, the nanoparticles have an average diameter of from about 30 nm to about 550 nm after reconstitution. In some embodiments, the nanoparticles have an average diameter of from about 30 nm to about 500 nm. In some embodiments, the nanoparticles have an average diameter of from about 30 nm to about 450 nm after reconstitution. In some embodiments, the nanoparticles have an average diameter of from about 30 nm to about 400 nm after reconstitution. In some embodiments, the nanoparticles have an average diameter of from about 30 nm to about 350 nm after reconstitution. In some embodiments, the nanoparticles have an average diameter of from about 30 nm to about 300 nm after reconstitution. In some embodiments, the nanoparticles have an average diameter of from about 30 nm to about 250 nm after reconstitution. In some embodiments, the nanoparticles have an average diameter of from about 30 nm to about 240 nm after reconstitution. In some embodiments, the nanoparticles have an average diameter of from about 30 nm to about 230 nm after reconstitution. In some embodiments, the nanoparticles have an average diameter of from about 30 nm to about 220 nm after reconstitution. In some embodiments, the nanoparticles have an average diameter of from about 30 nm to about 210 nm after reconstitution. In some embodiments, the nanoparticles have an average diameter of from about 30 nm to about 200 nm after reconstitution. In some embodiments, the nanoparticles have an average diameter of from about 30 nm to about 190 nm after reconstitution. In some embodiments, the nanoparticles have an average diameter of from about 30 nm to about 180 nm after reconstitution. In some embodiments, the nanoparticles have an average diameter of from about 30 nm to about 170 nm after reconstitution. In some embodiments, the nanoparticles have an average diameter of from about 30 nm to about 160 nm after reconstitution. In some embodiments, the nanoparticles have an average diameter of from about 30 nm to about 150 nm after reconstitution. In some embodiments, the nanoparticles have an average diameter of from about 30 nm to about 140 nm after reconstitution. In some embodiments, the nanoparticles have an average diameter of from about 30 nm to about 130 nm after reconstitution. In some embodiments, the nanoparticles have an average diameter of from about 30 nm to about 120 nm after reconstitution. In some embodiments, the nanoparticles have an average diameter of from about 30 nm to about 110 nm after reconstitution. In some embodiments, the nanoparticles have an average diameter of from about 30 nm to about 100 nm after reconstitution. In some embodiments, the nanoparticles have an average diameter of from about 30 nm to about 90 nm after reconstitution. In some embodiments, the nanoparticles have an average diameter of from about 30 nm to about 80 nm after reconstitution. In some embodiments, the nanoparticles have an average diameter of from about 30 nm to about 70 nm after reconstitution. In some embodiments, the nanoparticles have an average diameter of from about 30 nm to about 60 nm after reconstitution. In some embodiments, the nanoparticles have an average diameter of from about 30 nm to about 50 nm after reconstitution. In some embodiments, the nanoparticles have an average diameter of from about 30 nm to about 40 nm after reconstitution.

In some embodiments, the nanoparticles have an average diameter of from about 40 nm to about 1000 nm after reconstitution. In some embodiments, the nanoparticles have an average diameter of from about 40 nm to about 950 nm after reconstitution. In some embodiments, the nanoparticles have an average diameter of from about 40 nm to about 900 nm after reconstitution. In some embodiments, the nanoparticles have an average diameter of from about 40 nm to about 850 nm after reconstitution. In some embodiments, the nanoparticles have an average diameter of from about 40 nm to about 800 nm after reconstitution. In some embodiments, the nanoparticles have an average diameter of from about 40 nm to about 750 nm after reconstitution. In some embodiments, the nanoparticles have an average diameter of from about 40 nm to about 700 nm after reconstitution. In some embodiments, the nanoparticles have an average diameter of from about 40 nm to about 650 nm after reconstitution. In some embodiments, the nanoparticles have an average diameter of from about 40 nm to about 600 nm after reconstitution. In some embodiments, the nanoparticles have an average diameter of from about 40 nm to about 550 nm after reconstitution. In some embodiments, the nanoparticles have an average diameter of from about 40 nm to about 500 nm after reconstitution. In some embodiments, the nanoparticles have an average diameter of from about 40 nm to about 450 nm after reconstitution. In some embodiments, the nanoparticles have an average diameter of from about 40 nm to about 400 nm after reconstitution. In some embodiments, the nanoparticles have an average diameter of from about 40 nm to about 350 nm after reconstitution. In some embodiments, the nanoparticles have an average diameter of from about 40 nm to about 300 nm. In some embodiments, the nanoparticles have an average diameter of from about 40 nm to about 250 nm after reconstitution. In some embodiments, the nanoparticles have an average diameter of from about 40 nm to about 240 nm after reconstitution. In some embodiments, the nanoparticles have an average diameter of from about 40 nm to about 230 nm after reconstitution. In some embodiments, the nanoparticles have an average diameter of from about 40 nm to about 220 nm after reconstitution. In some embodiments, the nanoparticles have an average diameter of from about 40 nm to about 210 nm after reconstitution. In some embodiments, the nanoparticles have an average diameter of from about 40 nm to about 200 nm after reconstitution. In some embodiments, the nanoparticles have an average diameter of from about 40 nm to about 190 nm after reconstitution. In some embodiments, the nanoparticles have an average diameter of from about 40 nm to about 180 nm after reconstitution. In some embodiments, the nanoparticles have an average diameter of from about 40 nm to about 170 nm after reconstitution. In some embodiments, the nanoparticles have an average diameter of from about 40 nm to about 160 nm after reconstitution. In some embodiments, the nanoparticles have an average diameter of from about 40 nm to about 150 nm after reconstitution. In some embodiments, the nanoparticles have an average diameter of from about 40 nm to about 140 nm after reconstitution. In some embodiments, the nanoparticles have an average diameter of from about 40 nm to about 130 nm after reconstitution. In some embodiments, the nanoparticles have an average diameter of from about 40 nm to about 120 nm after reconstitution. In some embodiments, the nanoparticles have an average diameter of from about 40 nm to about 110 nm after reconstitution. In some embodiments, the nanoparticles have an average diameter of from about 40 nm to about 100 nm after reconstitution. In some embodiments, the nanoparticles have an average diameter of from about 40 nm to about 90 nm after reconstitution. In some embodiments, the nanoparticles have an average diameter of from about 40 nm to about 80 nm after reconstitution. In some embodiments, the nanoparticles have an average diameter of from about 40 nm to about 70 nm after reconstitution. In some embodiments, the nanoparticles have an average diameter of from about 40 nm to about 60 nm after reconstitution. In some embodiments, the nanoparticles have an average diameter of from about 40 nm to about 50 nm after reconstitution.

In some embodiments, the nanoparticles have an average diameter of from about 50 nm to about 1000 nm after reconstitution. In some embodiments, the nanoparticles have an average diameter of from about 50 nm to about 950 nm after reconstitution. In some embodiments, the nanoparticles have an average diameter of from about 50 nm to about 900 nm after reconstitution. In some embodiments, the nanoparticles have an average diameter of from about 50 nm to about 850 nm after reconstitution. In some embodiments, the nanoparticles have an average diameter of from about 50 nm to about 800 nm after reconstitution. In some embodiments, the nanoparticles have an average diameter of from about 50 nm to about 750 nm after reconstitution. In some embodiments, the nanoparticles have an average diameter of from about 50 nm to about 700 nm after reconstitution. In some embodiments, the nanoparticles have an average diameter of from about 50 nm to about 650 nm after reconstitution. In some embodiments, the nanoparticles have an average diameter of from about 50 nm to about 600 nm after reconstitution. In some embodiments, the nanoparticles have an average diameter of from about 50 nm to about 550 nm after reconstitution. In some embodiments, the nanoparticles have an average diameter of from about 50 nm to about 500 nm after reconstitution. In some embodiments, the nanoparticles have an average diameter of from about 50 nm to about 450 nm after reconstitution. In some embodiments, the nanoparticles have an average diameter of from about 50 nm to about 400 nm after reconstitution. In some embodiments, the nanoparticles have an average diameter of from about 50 nm to about 350 nm after reconstitution. In some embodiments, the nanoparticles have an average diameter of from about 50 nm to about 300 nm after reconstitution. In some embodiments, the nanoparticles have an average diameter of from about 50 nm to about 250 nm after reconstitution. In some embodiments, the nanoparticles have an average diameter of from about 50 nm to about 240 nm after reconstitution. In some embodiments, the nanoparticles have an average diameter of from about 50 nm to about 230 nm after reconstitution. In some embodiments, the nanoparticles have an average diameter of from about 50 nm to about 220 nm after reconstitution. In some embodiments, the nanoparticles have an average diameter of from about 50 nm to about 210 nm after reconstitution. In some embodiments, the nanoparticles have an average diameter of from about 50 nm to about 200 nm after reconstitution. In some embodiments, the nanoparticles have an average diameter of from about 50 nm to about 190 nm after reconstitution. In some embodiments, the nanoparticles have an average diameter of from about 50 nm to about 180 nm after reconstitution. In some embodiments, the nanoparticles have an average diameter of from about 50 nm to about 170 nm after reconstitution. In some embodiments, the nanoparticles have an average diameter of from about 50 nm to about 160 nm after reconstitution. In some embodiments, the nanoparticles have an average diameter of from about 50 nm to about 150 nm after reconstitution. In some embodiments, the nanoparticles have an average diameter of from about 50 nm to about 140 nm after reconstitution. In some embodiments, the nanoparticles have an average diameter of from about 50 nm to about 130 nm after reconstitution. In some embodiments, the nanoparticles have an average diameter of from about 50 nm to about 120 nm after reconstitution. In some embodiments, the nanoparticles have an average diameter of from about 50 nm to about 110 nm after reconstitution. In some embodiments, the nanoparticles have an average diameter of from about 50 nm to about 100 nm after reconstitution. In some embodiments, the nanoparticles have an average diameter of from about 50 nm to about 90 nm after reconstitution. In some embodiments, the nanoparticles have an average diameter of from about 50 nm to about 80 nm after reconstitution. In some embodiments, the nanoparticles have an average diameter of from about 50 nm to about 70 nm after reconstitution. In some embodiments, the nanoparticles have an average diameter of from about 50 nm to about 60 nm after reconstitution.

In some embodiments, the nanoparticles have an average diameter of about 10 nm after reconstitution. In some embodiments, the nanoparticles have an average diameter of about 20 nm after reconstitution. In some embodiments, the nanoparticles have an average diameter of about 30 nm after reconstitution. In some embodiments, the nanoparticles have an average diameter of about 40 nm after reconstitution. In some embodiments, the nanoparticles have an average diameter of about 50 nm after reconstitution. In some embodiments, the nanoparticles have an average diameter of about 60 nm after reconstitution. In some embodiments, the nanoparticles have an average diameter of about 70 nm after reconstitution. In some embodiments, the nanoparticles have an average diameter of about 80 nm after reconstitution. In some embodiments, the nanoparticles have an average diameter of about 90 nm after reconstitution. In some embodiments, the nanoparticles have an average diameter of about 100 nm after reconstitution. In some embodiments, the nanoparticles have an average diameter of about 110 nm after reconstitution. In some embodiments, the nanoparticles have an average diameter of about 120 nm after reconstitution. In some embodiments, the nanoparticles have an average diameter of about 130 nm after reconstitution. In some embodiments, the nanoparticles have an average diameter of about 140 nm after reconstitution. In some embodiments, the nanoparticles have an average diameter of about 150 nm after reconstitution. In some embodiments, the nanoparticles have an average diameter of about 160 nm after reconstitution. In some embodiments, the nanoparticles have an average diameter of about 170 nm after reconstitution. In some embodiments, the nanoparticles have an average diameter of about 180 nm. In some embodiments, the nanoparticles have an average diameter of about 190 nm after reconstitution. In some embodiments, the nanoparticles have an average diameter of about 200 nm after reconstitution. In some embodiments, the nanoparticles have an average diameter of about 210 nm after reconstitution. In some embodiments, the nanoparticles have an average diameter of about 220 nm after reconstitution. In some embodiments, the nanoparticles have an average diameter of about 230 nm after reconstitution. In some embodiments, the nanoparticles have an average diameter of about 240 nm after reconstitution. In some embodiments, the nanoparticles have an average diameter of about 250 nm after reconstitution. In some embodiments, the nanoparticles have an average diameter of about 300 nm after reconstitution. In some embodiments, the nanoparticles have an average diameter of about 350 nm after reconstitution. In some embodiments, the nanoparticles have an average diameter of about 400 nm after reconstitution. In some embodiments, the nanoparticles have an average diameter of about 450 nm after reconstitution. In some embodiments, the nanoparticles have an average diameter of about 500 nm after reconstitution. In some embodiments, the nanoparticles have an average diameter of about 550 nm after reconstitution. In some embodiments, the nanoparticles have an average diameter of about 600 nm after reconstitution. In some embodiments, the nanoparticles have an average diameter of about 650 nm after reconstitution. In some embodiments, the nanoparticles have an average diameter of about 700 nm after reconstitution. In some embodiments, the nanoparticles have an average diameter of about 750 nm after reconstitution. In some embodiments, the nanoparticles have an average diameter of about 800 nm. In some embodiments, the nanoparticles have an average diameter of about 850 nm after reconstitution. In some embodiments, the nanoparticles have an average diameter of about 900 nm after reconstitution. In some embodiments, the nanoparticles have an average diameter of about 950 nm after reconstitution. In some embodiments, the nanoparticles have an average diameter of about 1000 nm after reconstitution.

### Preparation of Nanoparticles

Provided in another aspect is a process of preparing any one of the compositions comprising the nanoparticles described herein, comprising:
a) dissolving a prodrug of a nucleoside or nucleotide in a volatile solvent to form a solution comprising a dissolved prodrug of a nucleoside or nucleotide;
b) adding the solution comprising the dissolved prodrug of a nucleoside or nucleotide to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form the any one of the compositions described herein.

In some embodiments, the adding the solution comprising the dissolved prodrug of a nucleoside or nucleotide to a pharmaceutically acceptable carrier in an aqueous solution from step b) further comprises mixing to form an emulsion. In some embodiments, the mixing is performed with a homogenizer. In some embodiments, the volatile solvent is a chlorinated solvent, alcohol, ketone, ester, ether, acetonitrile, or any combination thereof. In some embodiments, volatile solvent is a chlorinated solvent. Examples of chlorinated solvents include, but are not limited to, chloroform, dichloromethane, and 1,2, dichloroethane. In some embodiments, volatile solvent is an alcohol. Examples of alcohols, include but are not limited to, methanol, ethanol, butanol (such as t-butyl and n-butyl alcohol), and propanol (such as iso-propyl alcohol). In some embodiments, volatile solvent is a ketone. An example of a ketone includes, but is not limited to, acetone. In some embodiments, volatile solvent is an ester. An example of an ester includes, but is not limited to ethyl acetate. In some embodiments, volatile solvent is an ether. In some embodiments, the volatile solvent is acetonitrile. In some embodiments, the volatile solvent is mixture of a chlorinated solvent with an alcohol.

In some embodiments, the volatile solvent is chloroform, ethanol, butanol, methanol, propanol, or a combination thereof. In some embodiments, volatile solvent is a mixture of chloroform and ethanol. In some embodiments, the volatile solvent is methanol. In some embodiments, the volatile solvent is a mixture of chloroform and methanol. In some embodiments, the volatile solvent is butanol, such as t-butanol or n-butanol. In some embodiments, the volatile solvent is a mixture of chloroform and butanol. In some embodiments, the volatile solvent is acetone. In some embodiments, the volatile solvent is acetonitrile. In some embodiments, the volatile solvent is dichloromethane. In some embodiments, the volatile solvent is 1,2 dichloroethane. In some embodiments the volatile solvent is ethyl acetate. In some embodiments, the volatile solvent is isopropyl alcohol. In some embodiments, the volatile solvent is chloroform. In some embodiments, the volatile solvent is ethanol. In some embodiments, the volatile solvent is a combination of ethanol and chloroform.

In some embodiments, the homogenization is high pressure homogenization. In some embodiments, the emulsion is cycled through high pressure homogenization for an appropriate amount of cycles. In some embodiments, the appropriate amount of cycles is from about 2 to about 10 cycles. In some embodiments, the appropriate amount of cycles is about 1, about 2, about 3, about 4, about 5, about 6, about 7, about 8, about 9, or about 10 cycles.

In some embodiments, the evaporation is accomplished with suitable equipment known for this purpose. Such suitable equipment include, but not limited to, rotary evaporators, falling film evaporators, wiped film evaporators, spray driers, and the like that can be operated in batch mode or in continuous operation. In some embodiments, the evaporation is accomplished with a rotary evaporator. In some embodiments, the evaporation is under reduced pressure.

### Administration

In some embodiments, the composition is suitable for injection. In some embodiments, the composition is suitable for parenteral administration. Examples of parenteral administration include but are not limited to subcutaneous injections, intravenous, or intramuscular injections or infusion techniques. In some embodiments, the composition is suitable for intravenous administration.

In some embodiments, the composition is administered intraperitoneally, intraarterially, intrapulmonarily, orally, by inhalation, intravesicularly, intramuscularly, intratracheally, subcutaneously, intraocularly, intrathecally, intratumorally, or transdermally. In some embodiments, the composition is administered intravenously. In some embodiments, the composition is administered intraarterially. In some embodiments, the composition is administered intrapulmonarily. In some embodiments, the composition is administered orally. In some embodiments, the composition is administered by inhalation. In some embodiments, the composition is administered intravesicularly. In some embodiments, the composition is administered intramuscularly. In some embodiments, the composition is administered intratracheally. In some embodiments, the composition is administered subcutaneously. In some embodiments, the composition is administered intraocularly. In some embodiments, the composition is administered intrathecally. In some embodiments, the composition is administered transdermally.

### Methods

Also provided herein in another aspect is a method of treating a disease in a subject in need thereof comprising administering any one of the compositions described herein.

In embodiment, the disease is cancer. Examples of cancers, include but not limited to solid tumors (e.g., tumors of the lung, breast, colon, prostate, bladder, rectum, brain or endometrium), hematological malignancies (e.g., leukemias, lymphomas, myelomas), carcinomas (e.g. bladder carcinoma, renal carcinoma, breast carcinoma, colorectal carcinoma), neuroblastoma, or melanoma. Non-limiting examples of these cancers include cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), acute lymphocytic leukemia, acute nonlymphocytic leukemia, chronic lymphocytic leukemia, chronic myelogenous leukemia, Hodgkin's disease, non-Hodgkin's lymphoma, multiple myeloma, mesothelioma, childhood solid tumors such as brain neuroblastoma, retinoblastoma, Wilms' tumor, bone cancer and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, Kaposi's sarcoma, neuroblastoma and retinoblastoma. In some embodiments, the cancer is breast cancer, ovarian cancer, non-small cell lung cancer, pancreatic cancer, or bladder cancer.

In some embodiments, the disease is caused by an infection. In some embodiments, the infection is viral. Examples of viral infection include, but are not limited to, picornaviruses (poliovirus, coxsackievirus, hepatitis A virus, echovirus, human rhinovirus, cardioviruses (e.g.. mengovirus and encephalomyocarditis virus) and foot-and-mouth disease virus); immunodeficiency virus (e.g., HIV-1, HIV-2 and related viruses including FIV-1 and SIV-1); hepatitis B virus (HBV); papillomavirus; Epstein-Barr virus (EBV); T-cell leukemia virus, e.g., HTLV-I, HTLV-II and related viruses, including bovine leukemia virus (BLV) and simian T-cell leukemia virus (STLV-I); hepatitis C virus (HCV); cytomegalovirus (CMV); influenza virus; herpes simplex virus (HSV). In some embodiments, the viral infection is human immunodeficiency virus (HIV), hepatitis B virus (HBV), hepatitis C virus. (HCV), Epstein-Barr virus (EBV), cytomegalovirus (CMV), or herpes simplex virus (HSV).

In some embodiments, the prodrug of the nucleoside or nucleotide is derived from a nucleoside or nucleotide that is an anticancer agent. In some embodiments, the prodrug of the nucleoside or nucleotide is derived from a nucleoside or nucleotide that is an antiviral agent.

Also disclosed herein is a method of delivering a prodrug of a nucleoside or nucleotide to a subject in need thereof comprising administering any one of the compositions described herein.

Disclosed compositions are administered to patients (animals and humans) in need of such treatment in dosages that will provide optimal pharmaceutical efficacy. It will be appreciated that the dose required for use in any particular application will vary from patient to patient, not only with the particular composition selected, but also with the route of administration, the nature of the condition being treated, the age and condition of the patient, concurrent medication or special diets then being followed by the patient, and other factors, with the appropriate dosage ultimately being at the discretion of the attendant physician. For treating diseases noted above, a contemplated composition disclosed herein is administered orally, subcutaneously, topically, parenterally, by inhalation spray, or rectally in dosage unit formulations containing conventional non-toxic pharmaceutically acceptable carriers, adjuvants and vehicles. Parenteral administration include subcutaneous injections, intravenous, or intramuscular injections or infusion techniques.

The following examples are provided merely as illustrative of various embodiments and shall not be construed to limit the invention in any way.

### EXAMPLES

### List of Abbreviations

As used above, and throughout the description of the invention, the following abbreviations, unless otherwise indicated, shall be understood to have the following meanings:
ACN acetonitrile
Bn benzyl
BOC or Boc tert-butyl carbamate
DCC N,N′-dicyclohexylcarbodiimide
DCM dichloromethane
DIPEA N,N-diisopropylethylamine
DMAP 4-(N,N-dimethylamino)pyridine
DMF dimethylformamide
DMA N,N-dimethylacetamide
DMSO dimethylsulfoxide
equiv equivalent(s)
EDCI 1-(3-dimethylaminopropyl)-3-ethylcarbodiimide
Et ethyl
EtOH ethanol
EtOAc ethyl acetate
HF hydrofluoric acid
HMDS bis(trimethylsilyl)amine
HPLC high performance liquid chromatography
Me methyl
MeOH methanol
MMTr 4-methoxytrityl
MMTrCl 4-methoxytrityl chloride
MS mass spectroscopy
NMM N-methylmorpholine
NMR nuclear magnetic resonance
rt room temperature
TBHP tert-butyl hydroperoxide
TEA triethylamine
TFA trifluoroacetic acid
THE tetrahydrofuran
TLC thin layer chromatography
TBDMSCI tert-butyldimethylsilyl chloride
TMSCI trimethylsilyl chloride
TMSOTf trimethylsilyl trifluoromethanesulfonate

The compounds used in the following examples and corresponding confirmation by mass spectrometry are shown in the following table.

| | Molecular | Exact | M/z |
|---|---|---|---|
| Compound | weight | mass | [M + 1]⁺ |
| 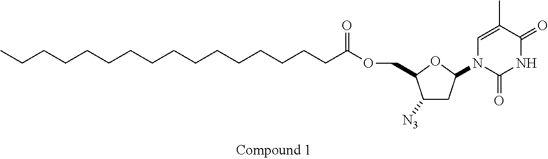 [SMILES: CCCCCCCCCCCCCCCCC(=O)OC[C@H]1O[C@@H](n2cc(C)c(=O)[nH]c2=O)C[C@@H]1N=[N+]=[N-]] | 519.69 | 519.34 | 520.14 |
| 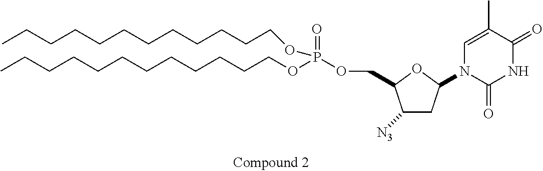 [SMILES: CCCCCCCCCCCCOP(=O)(OCCCCCCCCCCCC)OC[C@H]1O[C@@H](n2cc(C)c(=O)[nH]c2=O)C[C@@H]1N=[N+]=[N-]] | 683.87 | 683.44 | 684.61 |
| 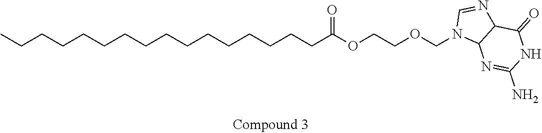 [SMILES: CCCCCCCCCCCCCCCCC(=O)OCCOCN1C=NC2C(=O)NC(N)=NC21] | 479.67 | 479.35 | 478.50 |
| 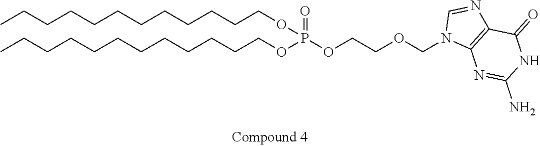 [SMILES: CCCCCCCCCCCCOP(=O)(OCCCCCCCCCCCC)OCCOCn1cnc2c(=O)[nH]c(N)nc21] | 641.83 | 641.43 | 642.62 |
| 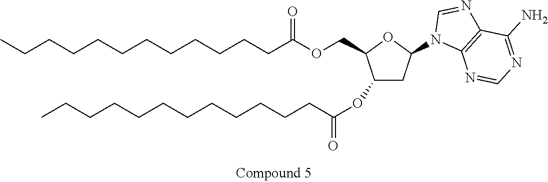 [SMILES: CCCCCCCCCCCCC(=O)OC[C@H]1O[C@@H](n2cnc3c(N)ncnc32)C[C@@H]1OC(=O)CCCCCCCCCCCC] | 643.91 | 643.47 | 644.5 |
| 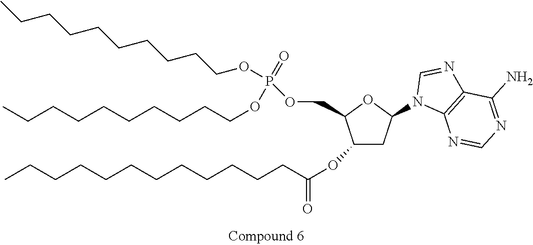 [SMILES: CCCCCCCCCCCCC(=O)O[C@H]1C[C@H](n2cnc3c(N)ncnc32)O[C@@H]1COP(=O)(OCCCCCCCCCC)OCCCCCCCCCC] | 808.1 | 807.56 | 808.49 |
| 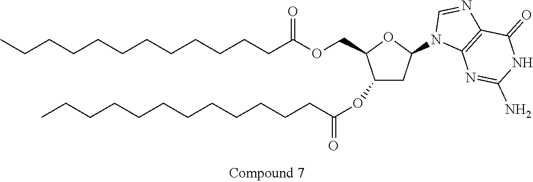 [SMILES: CCCCCCCCCCCCC(=O)OC[C@H]1O[C@@H](n2cnc3c(=O)[nH]c(N)nc32)C[C@@H]1OC(=O)CCCCCCCCCCCC] | 659.91 | 659.46 | 660.6 |
| 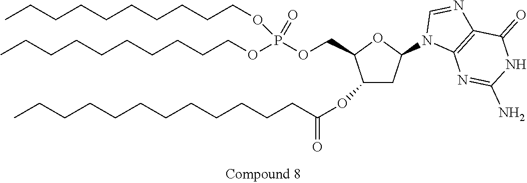 [SMILES: CCCCCCCCCCCCC(=O)O[C@H]1C[C@H](n2cnc3c(=O)[nH]c(N)nc32)O[C@@H]1COP(=O)(OCCCCCCCCCC)OCCCCCCCCCC] | 824.1 | 823.56 | 824.5 |
| 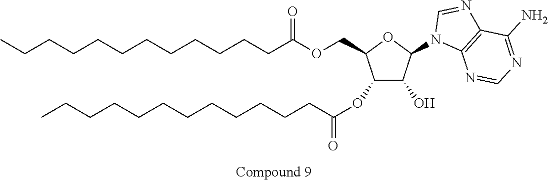 [SMILES: CCCCCCCCCCCCC(=O)OC[C@H]1O[C@@H](n2cnc3c(N)ncnc32)[C@H](O)[C@@H]1OC(=O)CCCCCCCCCCCC] | 659.91 | 659.46 | 660.71 |
| 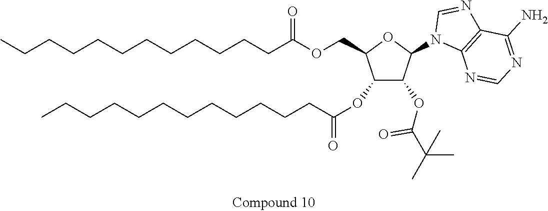 [SMILES: CCCCCCCCCCCCC(=O)OC[C@H]1O[C@@H](n2cnc3c(N)ncnc32)[C@H](OC(=O)C(C)(C)C)[C@@H]1OC(=O)CCCCCCCCCCCC] | 744.03 | 743.52 | 744.67 |
| 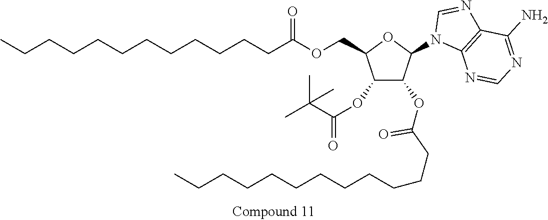 [SMILES: CCCCCCCCCCCCC(=O)OC[C@H]1O[C@@H](n2cnc3c(N)ncnc32)[C@H](OC(=O)CCCCCCCCCCCC)[C@@H]1OC(=O)C(C)(C)C] | 744.03 | 743.52 | 744.6 |
| 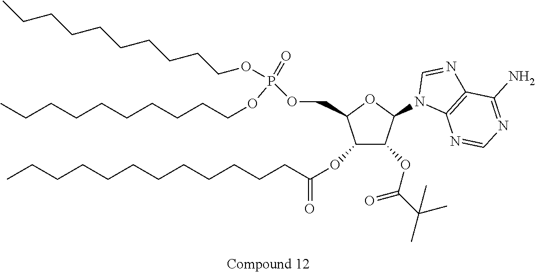 [SMILES: CCCCCCCCCCCCC(=O)O[C@H]1[C@@H](OC(=O)C(C)(C)C)[C@H](n2cnc3c(N)ncnc32)O[C@@H]1COP(=O)(OCCCCCCCCCC)OCCCCCCCCCC] | 908.22 | 907.62 | 908.63 |

### Examples of Formulating Albumin Nanoparticle Compositions with Nucleosides and Nucleotides

### Example 1: Nanoparticle Pharmaceutical Composition Comprising Compound 1 and Albumin

39.2 mL of a human albumin solution (1.47% w/v) was prepared diluting from a 25% human albumin U.S.P. solution using chloroform saturated water. Compound 1 (36 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 69 nm initially, 75 nm after 15 minutes, 100 nm after 4 hours, and 131 nm after 24 hours at room temperature.

### Example 2: Nanoparticle Pharmaceutical Composition Comprising Compound 2 and Albumin

39.2 mL of a human albumin solution (1.47% w/v) was prepared diluting from a 25% human albumin U.S.P. solution using chloroform saturated water. Compound 2 (46 mg) was dissolved in 800 μL chloroform/ethanol. The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 63 nm initially, 63 nm after 2 hours, 64 nm after 4 hours, and 68 nm after 24 hours at room temperature.

### Example 3: Nanoparticle Pharmaceutical Composition Comprising Compound 3 and Albumin

39.4 mL of a human albumin solution (1.47% w/v) was prepared diluting from a 25% human albumin U.S.P. solution using chloroform saturated water. Compound 3 (25 mg) was dissolved in 600 μL chloroform/ethanol (85:15 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 5 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The average particle size (Zₐᵥ, Malvern Nano-S) was determined to be 275 nm initially, 283 nm after 60 minutes, and 283 nm after 24 hours at room temperature.

### Example 4: Nanoparticle Pharmaceutical Composition Comprising Compound 4 and Albumin

39.4 mL of a human albumin solution (1.47% w/v) was prepared diluting from a 25% human albumin U.S.P. solution using chloroform saturated water. Compound 4 (33 mg) was dissolved in 600 μL chloroform/ethanol (85:15 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 5 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The average particle size (Zₐᵥ, Malvern Nano-S) was determined to be 200 nm initially, 199 nm after 120 minutes, and 207 nm after 24 hours at room temperature.

### Example 5: Nanoparticle Pharmaceutical Composition Comprising Compound 5 and Albumin

39.2 mL of a human albumin solution (1.47% w/v) was prepared diluting from a 25% human albumin U.S.P. solution using chloroform saturated water. Compound 5 (44 mg) was dissolved in 800 μL chloroform/ethanol (90/10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 75 nm initially, 81 nm after 120 minutes, and 102 nm after 3 days at room temperature.

### Example 6: Nanoparticle Pharmaceutical Composition Comprising Compound 6 and Albumin

39.2 mL of a human albumin solution (1.47% w/v) was prepared diluting from a 25% human albumin U.S.P. solution using chloroform saturated water. Compound 6 (56 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 61 nm initially, 64 nm after 240 minutes, and 81 nm after 33 days at room temperature.

### Example 7: Nanoparticle Pharmaceutical Composition Comprising Compound 7 and Albumin

39.2 mL of a human albumin solution (1.47% w/v) was prepared diluting from a 25% human albumin U.S.P. solution using chloroform saturated water. Compound 7 (48 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 70 nm initially, 69 nm after 240 minutes, and 71 nm after 24 hours at room temperature.

### Example 8: Nanoparticle Pharmaceutical Composition Comprising Compound 8 and Albumin

39.2 mL of a human albumin solution (1.47% w/v) was prepared diluting from a 25% human albumin U.S.P. solution using chloroform saturated water. Compound 8 (56 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 58 nm initially, 58 nm after 120 minutes, and 57 nm after 24 hours at room temperature.

### Example 9: Nanoparticle Pharmaceutical Composition Comprising Compound 9 and Albumin

39.2 mL of a human albumin solution (1.47% w/v) was prepared diluting from a 25% human albumin U.S.P. solution using chloroform saturated water. Compound 9 (45 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 91 nm initially, 99 nm after 30 minutes, 146 nm after 120 minutes, and 182 nm after 24 hours at room temperature.

### Example 10: Nanoparticle Pharmaceutical Composition Comprising Compound 10 and Albumin

39.2 mL of a human albumin solution (1.47% w/v) was prepared diluting from a 25% human albumin U.S.P. solution using chloroform saturated water. Compound 10 (50 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 67 nm initially, 67 nm after 240 minutes, and 67 nm after 24 hours at room temperature.

### Example 11: Nanoparticle Pharmaceutical Composition Comprising Compound 11 and Albumin

39.2 mL of a human albumin solution (1.47% w/v) was prepared diluting from a 25% human albumin U.S.P. solution using chloroform saturated water. Compound 11 (51 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 70 nm initially, 71 nm after 120 minutes, and 69 nm after 24 hours at room temperature.

### Example 12: Nanoparticle Pharmaceutical Composition Comprising Compound 12 and Albumin

39.2 mL of a human albumin solution (1.47% w/v) was prepared diluting from a 25% human albumin U.S.P. solution using chloroform saturated water. Compound 12 (62 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 64 nm initially, 64 nm after 120 minutes, and 64 nm after 24 hours at room temperature.

### Exemplary Nanoparticle Compositions Upon Lyophilization and Rehydration

### Example 13

This example demonstrates the lyophilization and rehydration into each of: water, 5% dextrose water, and saline for a nanoparticle pharmaceutical composition comprising Compound 2 and albumin. Immediately after sterile filtration, the nanoparticle suspension from Example 2 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 88 nm initially, 85 nm after 30 minutes, and 86 nm after 2 hours at room temperature. Upon hydration into 5% dextrose water, the average particle size (Zₐᵥ, Malvern Nano-S) was determined to be 98 nm initially, 95 nm after 30 minutes, and 95 nm after 2 hours at room temperature. Upon hydration into 0.9% saline, the average particle size (Zₐᵥ, Malvern Nano-S) was determined to be 85 nm initially, 87 nm after 30 minutes, and 94 nm after 2 hours at room temperature.

### Example 14

This example demonstrates the lyophilization and rehydration into each of: water, 5% dextrose water, and saline for a nanoparticle pharmaceutical composition comprising Compound 7 and albumin. Immediately after sterile filtration, the nanoparticle suspension from Example 7 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 69 nm initially, 68 nm after 30 minutes, and 68 nm after 2 hours at room temperature. Upon hydration into 5% dextrose water, the average particle size (Zₐᵥ, Malvern Nano-S) was determined to be 78 nm initially, 78 nm after 30 minutes, and 78 nm after 2 hours at room temperature. Upon hydration into 0.9% saline, the average particle size (Zₐᵥ, Malvern Nano-S) was determined to be 64 nm initially, 65 nm after 30 minutes, and 65 nm after 2 hours at room temperature.

### Example 15

This example demonstrates the lyophilization and rehydration into each of: water, 5% dextrose water, and saline for a nanoparticle pharmaceutical composition comprising Compound 10 and albumin. Immediately after sterile filtration, the nanoparticle suspension from Example 10 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 76 nm initially, 76 nm after 30 minutes, and 75 nm after 2 hours at room temperature. Upon hydration into 5% dextrose water, the average particle size (Zₐᵥ, Malvern Nano-S) was determined to be 86 nm initially, 85 nm after 30 minutes, and 86 nm after 2 hours at room temperature. Upon hydration into 0.9% saline, the average particle size (Zₐᵥ, Malvern Nano-S) was determined to be 76 nm initially, 87 nm after 30 minutes, and 97 nm after 2 hours at room temperature.

### Example 16

This example demonstrates the lyophilization and rehydration into each of water, 5% dextrose water, and saline for a nanoparticle pharmaceutical composition comprising Compound 12 and albumin. Immediately after sterile filtration, the nanoparticle suspension from Example 12 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 74 nm initially, 73 nm after 30 minutes, and 73 nm after 2 hours at room temperature. Upon hydration into 5% dextrose water, the average particle size (Zₐᵥ, Malvern Nano-S) was determined to be 83 nm initially, 83 nm after 30 minutes, and 82 nm after 2 hours at room temperature. Upon hydration into 0.9% saline, the average particle size (Zₐᵥ, Malvern Nano-S) was determined to be 71 nm initially, 79 nm after 30 minutes, and 88 nm after 2 hours at room temperature.

### Examples of No Albumin Nanoparticles Produced When Using Unmodified Nucleosides

### Examples 17-23

Unmodified nucleosides were not sufficiently soluble in most of the volatile organic solvents above to form a concentrated solution for addition to the albumin solution and homegenization. Therefore, nucleoside solubilization into a concentrated organic solution or near organic solution was completed as described in the table below. Other than the solvents used to solubilize the nucleosides and the unmodified nucleosides themselves as described in the table below, the same process as in Examples 5-12 above was used to attempt a preparation of a nanoparticle pharmaceutical composition of each dissolved nucleoside.

| | Unmodified | Amount of | | Solvent |
|---|---|---|---|---|
| | Nucleoside | Nucleoside | Solvent (ratio) | Volume |
| Example 17 | azidothymidine | 18 mg | ethanol/water (90:10) | 800 μL |
| Example 18 | thymidine | 19 mg | methanol/chloroform (50:50) | 1200 μL |
| Example 19 | 2′deoxyguanosine | 18 mg | methanol/water (75:25) | 1200 μL |
| | hydrate | | | |
| Example 20 | adenosine | 18 mg | water (100) | 1600 μL |
| Example 21 | 2′deoxyuridine | 16 mg | methanol/chloroform (50:50) | 800 μL |
| Example 22 | 5′bromo- | 21 mg | ethanol/water (90:10) | 800 μL |
| | 2′deoxyuridine | | | |
| Example 23 | cytidine | 19 mg | ethanol/water (75:25) | 1200 μL |

In each case of examples 17, 18, 19, 20, 21, 22, and 23, no nanoparticle formation was observed, with a measured average particle size in all cases (Zₐᵥ, Malvern Nano-S) less than 10 nm, consistent with monomeric albumin.

### Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants (J) are reported in Hertz. For proton spectra the solvent peak was used as the reference peak.

### Example 24: Synthesis of Compound 1

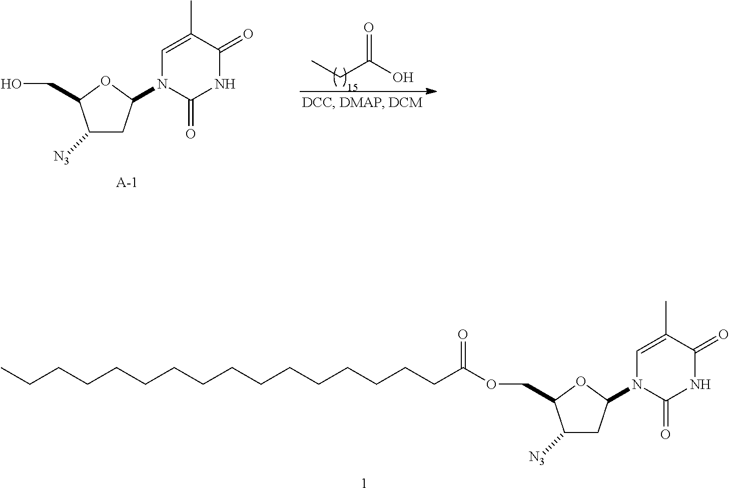

To a solution of A-1 (1.0 g, 0.0037 mol) in DCM (40 mL) was added TEA (1.89 g, 0.0186 mol), DCC (2.3 g, 0.0112 mol), and DMAP (0.0456 g, 0.00037 mol), followed by heptadecanoic acid (3.0 g, 0.0112 mol). The reaction mixture was stirred at room temperature for 16 h. The solvent was removed under reduced pressure. The residue was dissolved in ethyl acetate (100 mL), and washed with water (40 mL) and brine (40 mL). The organic layer was dried over anhydrous Na₂SO₄, filtered, and concentrated under reduced pressure. The crude compound was purified by silica gel (100-200 mesh) chromatography to afford Compound 1 (0.321 g, 8%) as an off-white solid. MS (ESI) m/z 520.14 [M+H]⁺; H NMR (400 MHz, DMSO-d₆) δ 11.36 (s, 1H), 7.45 (s, 1H), 6.12 (m, 1H), 4.48-4.44 (m, 1H), 4.28-4.22 (m, 2H), 3.99-3.95 (m, 1H), 2.48-2.42 (m, 1H), 2.36-2.29 (m, 3H), 1.54-1.51 (m, 2H), 1.23 (m, 26H), 0.87-0.83 (m, 3H).

### Example 25: Synthesis of Compound 2

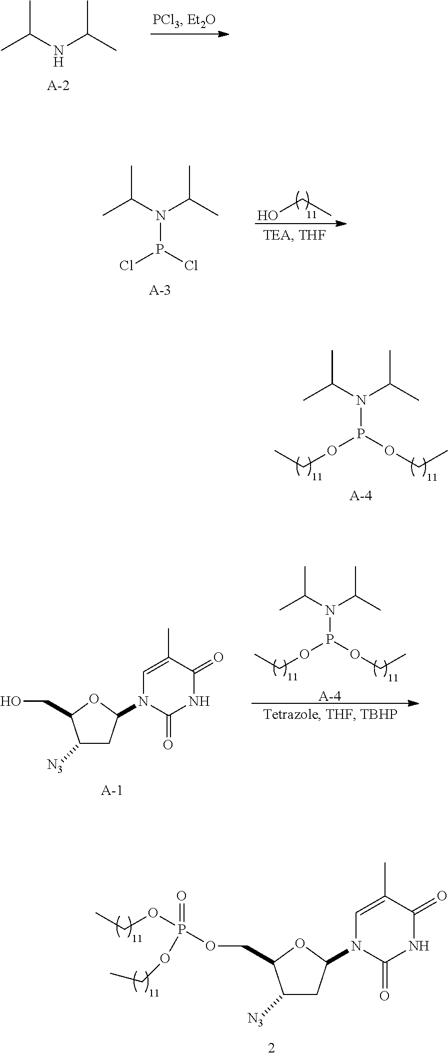

### Synthesis of A-4

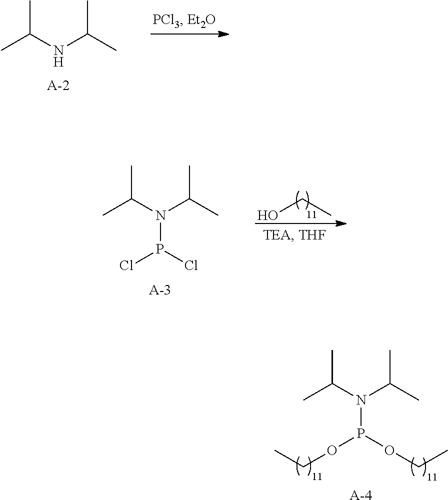

To a stirred solution of PCl₃ (10 g, 0.0728 mol) in THF (300 mL) was added diisopropyl amine (14.7 g, 0.145 mol) in THF (60 mL) dropwise over a period of 1 h at 0° C. The reaction mixture was stirred at rt for 4 h under argon atmosphere. The reaction mixture was concentrated under reduced pressure to get crude compound A-3 (10.0 g) which was used directly in the next step without further purification.

To a stirred solution of A-3 (10.0 g, 0.0497 mol) in THF (300 mL) were added TEA (10.57 g, 0.104 mol) followed by 1-dodecanol (18.5 g, 0.0994 mol) dropwise over a period of 1 h at 0° C. The resulting reaction mixture was allowed to room temperature over 2 h. After 2 h, the reaction mixture was concentrated under reduced pressure to afford compound A-4 (17.0 g) which was used directly in the next step without further purification.

### Synthesis of Compound 2

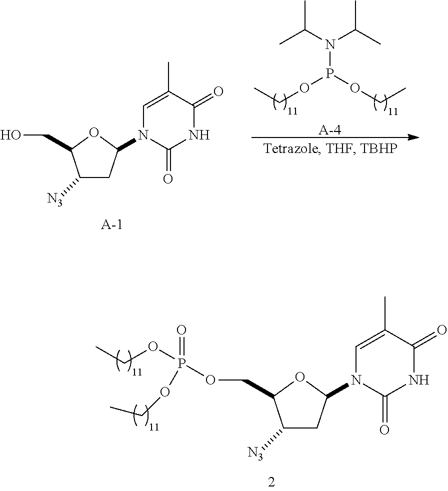

To a stirred solution of A-4 (1.12 g, 0.00224 mol) in dry THF (12 mL) was added tetrazole (0.472 g, 0.0067 mol) at −30° C. To the resulting reaction mixture was added a solution of A-1 (0.6 g, 0.00224 mol) in THF (16 mL) dropwise at −30° C. for 30 min. The reaction mixture was stirred at room temperature for 3 h. tert-Butyl hydrogen peroxide in decane (1.3 mL, 5M) was then added dropwise at −30° C. and the reaction mixture stirred for 3 h at −30° C. The reaction mixture was then diluted with ethyl acetate (50 mL). The organic layer washed with water (20 mL) and brine (20 mL) solution. The organic layer was dried over anhydrous Na₂SO₄, filtered, and concentrated under reduced pressure. The residue was purified by silica gel (100-200 mesh) chromatography to afford Compound 2 (0.245 g, 16%) as a colorless liquid. MS (ESI) m/z 684.61 [M+H]⁺; H NMR (400 MHz, DMSO-d₆) δ 11.37 (s, 1H), 7.50 (s, 1H), 6.14 (m, 1H), 4.48-4.44 (m, 1H), 4.19-4.13 (m, 2H), 3.99-3.95 (m, 5H), 2.44-2.30 (m, 2H), 1.79 (m, 3H), 1.62-1.55 (m, 4H), 1.27-1.23 (m, 36H), 0.87-0.83 (m, 6H).

### Example 26: Synthesis of Compound 3

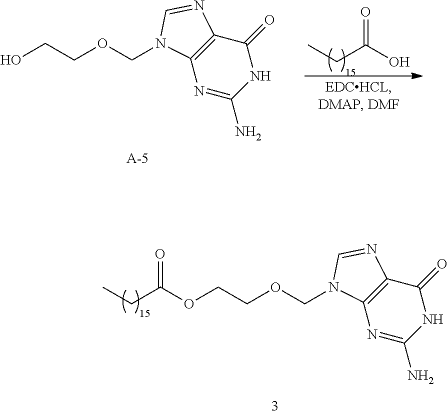

To a stirred solution of heptadecanoic acid (2.0 g, 0.0073 mol) in DMF (20 mL) was added DMAP (1.35 g, 0.011 mol) and EDC.HCl (2.83 g, 0.0147 mol) at 0° C. The resulting reaction mixture was stirred for 1 h at rt, and then compound A-5 (3.5 g, 0.0188 mol) was added slowly at 0° C. The resulting reaction mixture was stirred at room temperature for 72 h. The reaction mass was filtered and the solvent was evaporated under reduced pressure. The residue was purified by silica gel (100-200 mesh) chromatography to afford Compound 3 (0.343 g, 10%) as an off-white solid. MS (ESI) m/z 478.50 [M+H]⁺; ¹H NMR (400 MHz, DMSO-d₆) δ 10.62 (s, 1H), 7.81 (s, 1H), 6.50 (s, 2H), 5.34 (s, 2H), 4.09-4.07 (m, 2H), 3.66-3.64 (m, 2H), 2.23-2.20 (m, 2H), 1.47-1.44 (m, 2H), 1.23 (m, 26H), 0.87-0.83 (m, 3H).

### Example 27: Synthesis of Compound 4

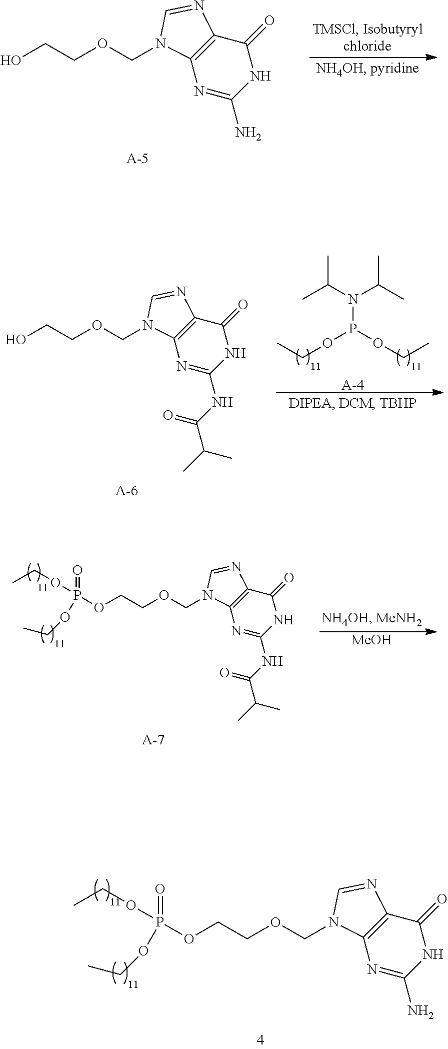

To a suspension of A-5 (5 g, 0.022 mol) in dry pyridine (125 mL) under a nitrogen atmosphere was added trichloromethylsilane (18.1 g, 0.166 mol). The reaction mixture was stirred at ambient temperature for 2 hours and cooled to 0° C. Isobutyrylchloride (7.1 g, 0.066 mol) was then added dropwise over 15 min. The mixture was allowed to warm to rt stirred for 3 hours. The reaction mixture was cooled to 0° C. and quenched by addition of water (15 mL). After stirring the mixture for 5 min at 0° C. and then 5 min at rt, concentrated aq. ammonium hydroxide (32.5 mL) was added. After stirring for an additional 15 min at rt, the mixture was diluted with water (250 mL) and washed with DCM (100 mL). The aq. layer was evaporated under reduced pressure at 45° C. The residue was co-evaporated with toluene three times. The crude compound purified by silica gel (100-200 mesh) chromatography to afford A-6 (3.5 g, 53%) as a light yellow solid.

To a stirred solution of A-6 (3.0 g, 0.010 mol) in DCM (90 mL) was added DIPEA (6.57 g, 0.051 mol) followed by tetrazole (1.78 g, 0.025 mol) at 0° C. To the resulting reaction mixture was added a solution of A-4 (15.2 g, 0.03 mol) in DCM (30 mL) dropwise at 0° C. for 15 min. The reaction mixture was stirred at room temperature for 16 h. tert-Butyl hydrogen peroxide in decane (6.0 mL, 5M) was added dropwise at 0° C. and the reaction mixture stirred for 6 h at room temperature. The reaction mixture was then concentrated at 40° C. and diluted with ethyl acetate (60 mL). The organic layer washed with water (20 mL) and brine (20 mL) solution. The organic layer was dried over anhydrous Na₂SO₄, filtered, and concentrated under reduced pressure. The residue was purified by silica gel (100-200 mesh) chromatography to afford A-7 (2.0 g, 28%) as a red liquid.

To a solution of A-7 (2.0 g, 0.0028 mmol) in methanol (40 mL) was added aq. NH₃ (12 mL, 6 v) followed by MeNH₂ 2M in THE (21.6 mL, 5.4 v) dropwise at 0° C. The reaction mixture was slowly warm to room temperature and stirred for 12 h at room temperature. The solvent was then evaporated under reduced pressure. The residue was dissolved in ethyl acetate (500 mL) and washed with water (100 mL) and brine (100 mL). The organic layer was dried over anhydrous Na₂SO₄, filtered, and concentrated. The crude compound was purified by silica gel (100-200 mesh) chromatography to afford Compound 4 (0.358 g, 20%) as an off-white solid. MS (ESI) m/z 642.62 [M+H]⁺; H NMR (400 MHz, DMSO-d₆) δ 10.61 (s, 1H), 7.81 (s, 1H), 6.50 (s, 2H), 5.36 (s, 2H), 4.04-4.00 (m, 2H), 3.91-3.85 (m, 4H), 3.67-3.65 (m, 2H), 1.56-1.51 (m, 4H), 1.23 (m, 36H), 0.87-0.83 (m, 6H).

### Example 28: Synthesis of Compound 5

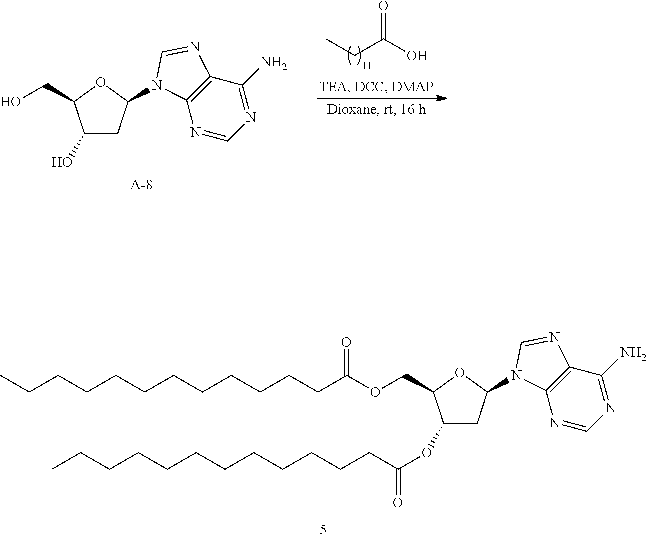

To (2R,3S,5R)-5-(6-amino-9H-purin-9-yl)-2-(hydroxymethyl)tetrahydrofuran-3-ol (A-8) (1 g, 3.984 mmol) in 1,4-dioxane (20 mL, 20 vol) at rt was added tridecanoic acid (2.6 g, 11.95 mmol), TEA (2.8 mL, 19.9 mmol), DCC (2.5 g, 11.95 mmol) and DMAP (49 mg, 0.398 mmol. The reaction mixture was stirred at rt for 16 h. The solvent was then evaporated, and the residue was taken in water (100 mL) and extracted with ethyl acetate (4×50 mL). The combined organic layer was washed with water (50 mL), brine (50 mL), dried over anhydrous Na₂SO₄, filtered, and concentrated under reduced pressure. The residue was purified by silica gel chromatography (100-200 mesh) and prep HPLC to afford (2R,3S,5R)-5-(6-amino-9H-purin-9-yl)-2-((tridecanoyloxy)methyl)tetrahydrofuran-3-yl tridecanoate (Compound 5) (600 mg, 23%) as a white solid. MS (ESI) m/z 644.5 [M+H]⁺; H NMR (400 MHz, DMSO-d₆) δ 8.32 (s, 1H), 8.14 (s, 1H), 7.30 (br s, 2H), 6.36 (t, J=6.4 Hz, 1H), 5.44-5.40 (m, 1H), 4.35-4.28 (m, 1H), 4.26-4.17 (m, 2H), 3.18-3.11 (m, 1H), 2.45-2.50 (m, 1H), 2.36 (t, J=7.6 Hz, 2H), 2.30-2.26 (m, 2H), 1.57-1.46 (m, 4H), 1.35-1.15 (m, 36H), 0.84 (t, J=5.2 Hz, 6H).

### Example 29: Synthesis of Compound 6

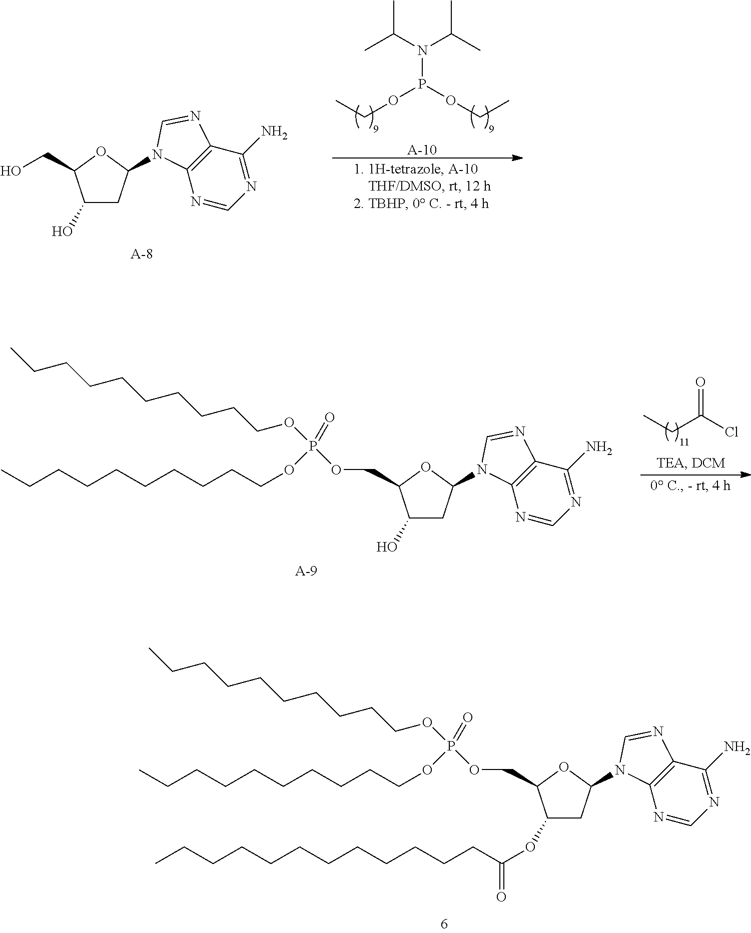

### Synthesis of A-10

To a stirred solution of N,N-diisopropylphosphoramide dichloridite (A-3) (20 g, 98.98 mmol) in dry THE (100 mL) were added TEA (29.16 mL, 207.86 mmol) followed by decanol (37.8 mL, 107.9 mmol) dropwise at 0° C. The reaction mixture was stirred for 1 h at 0° C. and then warmed to rt and stirred for 3 h. The reaction mixture was filtered through a celite pad and the precipitated solid was washed with dry THF. The filtrate was concentrated argon atmosphere to afford didecyl diisopropylphosphoramidite (A-10) (38 g, 79%) as a colorless liquid.

### Synthesis of Compound 6

To a stirred solution of A-8 (7 g, 27.88 mmol) in THF/DMSO (1:1, 140 mL) were added 1H-tetrazole (5.85 g, 83.64 mmol) followed by didecyl diisopropylphosphoramidite (A-10) (14.89 g, 33.46 mmol) at 0° C. The reaction mixture was stirred at rt for 12 h and then 5M TBHP in decane (16.72 mL, 83.64 mmol) at 0° C. was added. The reaction mixture was stirred at rt for 4 h. The reaction was then quenched with saturated NaHSO₃ solution and extracted with EtOAc (2×150 mL). The combined organic layers were washed with saturated NaHSO₃ solution (2×100 mL), brine (2×100 mL), dried over Na₂SO₄, filtered, and concentrated under reduced pressure. The residue was purified by silica gel chromatography to afford ((2R,3S,5R)-5-(6-amino-9H-purin-9-yl)-3-hydroxytetrahydrofuran-2-yl) methyl didecyl phosphate (A-9) (2 g, 9%) as a liquid.

To a stirred solution of ((2R,3S,5R)-5-(6-amino-9H-purin-9-yl)-3-hydroxytetrahydrofuran-2-yl) methyl didecyl phosphate (A-9) (1 g, 1.63 mmol) in DCM (20 mL) were added TEA (0.68 mL, 4.90 mmol) followed by tridecanoyl chloride (3) (0.45 mL, 1.96 mmol) at 0° C. The reaction mixture was stirred at rt for 4 h. The reaction was then quenched with ice cold water and extracted with EtOAc (2×30 mL). The combined organic layers were washed with brine (30 mL), dried over Na₂SO₄, filtered, and concentrated under reduced pressure. The residue was purified by prep-TLC to afford (2R,3S,5R)-5-(6-amino-9H-purin-9-yl)-2-(((bis(decyloxy)phosphoryl)oxy)methyl) tetrahydrofuran-3-yl tridecanoate (Compound 6) (200 mg, 15%) as a liquid. MS (ESI) m/z 808.49 [M+H]⁺; H NMR (400 MHz, CDCl₃) δ 8.35 (s, 1H), 8.17 (s, 1H), 6.51 (dd, J=8.8, 5.6 Hz, 1H), 5.88 (br s, 2H), 5.46 (d, J=5.6 Hz, 1H), 4.32 (dd, J=11.6, 6.4 Hz, 3H), 4.03 (q, J=5.6 Hz, 4H), 2.81 (dd, J=14.4, 8.8 Hz, 1H), 2.59 (dd, J=14.4, 5.2 Hz, 1H), 2.36 (t, J=7.6 Hz, 2H), 1.66-1.63 (m, 6H), 1.45-1.24 (m, 48H) 0.75 (t, J=5.2 Hz, 9H).

### Example 30: Synthesis of Compound 7

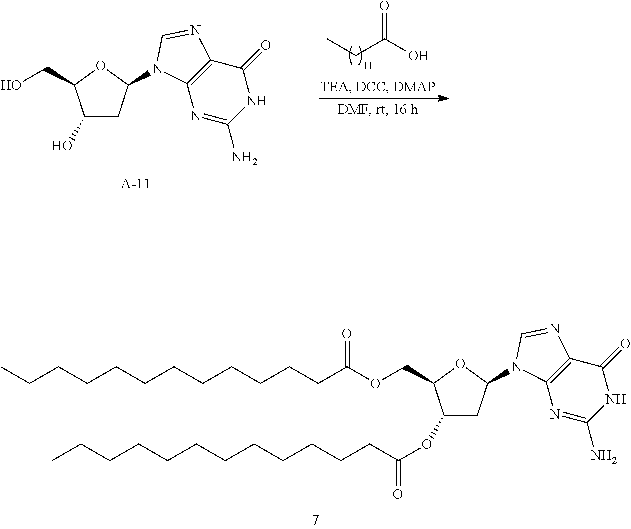

To 2-amino-9-((2R,4S,5R)-4-hydroxy-5-(hydroxymethyl)tetrahydrofuran-2-yl)-1,9-dihydro-6H-purin-6-one A-11 (2.5 g, 9.36 mmol) in DMF (100 mL, 20 vol) at rt was added tridecanoic acid (10 g, 46.82 mmol), TEA (6.6 mL, 46.82 mmol), DCC (9.65 g, 46.816 mmol) and DMAP (1.14 g, 9.36 mmol). The reaction mixture was stirred at rt for 16 h. The solvent was then evaporated, and the residue was taken in water (200 mL) and extracted with ethyl acetate (4×10 mL). The combined organic layer was washed with water (150 mL), brine (150 mL), dried over anhydrous Na₂SO₄, filtered, and concentrated under reduced pressure. The residue was purified by silica gel (100-200 mesh) chromatography and further purified by trituration with MeOH to afford (2R,3S,5R)-5-(2-amino-6-oxo-1,6-dihydro-9H-purin-9-yl)-2-((tridecanoyloxy)methyl)tetrahydrofuran-3-yl tridecanoate (Compound 7) (681 mg, 23%) as a white solid. MS (ESI) m/z 660.6 [M+H]⁺; ¹H NMR (400 MHz, CDCl₃) δ 12.1 (br s, 1H), 7.71 (s, 1H), 6.32-6.2 (m, 3H), 5.39 (d, J=6 Hz, 1H), 4.46-4.28 (m, 3H), 2.9-2.82 (m, 1H), 2.55-2.48 (m, 1H), 2.35 (q, J=7.2 Hz, 1H), 1.69-1.56 (m, 4H), 1.38-1.15 (m, 37H), 0.91-0.84 (m, 6H).

### Example 31: Synthesis of Compound 8

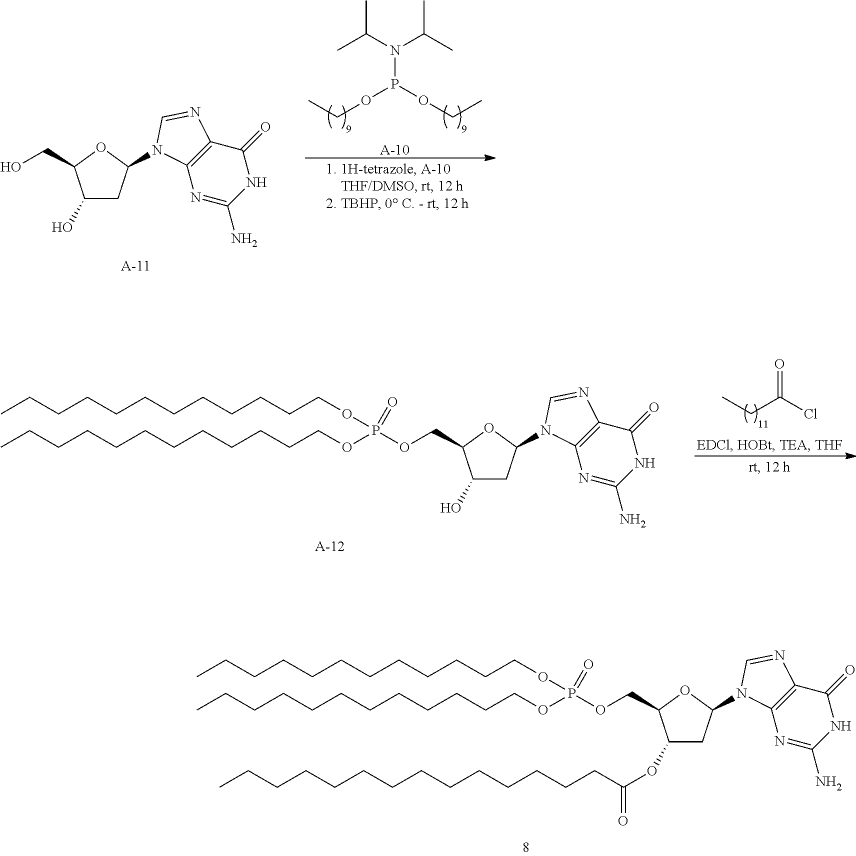

To a stirred solution of 2-amino-9-((2R,4S,5R)-4-hydroxy-5-(hydroxymethyl)tetrahydrofuran-2-yl)-1,9-dihydro-6H-purin-6-one (A-11) (7 g, 26.21 mmol) in THF/DMSO (1:1, 140 mL) was added 1H-tetrazole (5.5 g, 78.63 mmol) followed by didecyl diisopropylphosphoramidite (A-10) (13.90 g, 31.46 mmol) at 0° C. The reaction mixture was stirred at rt for 12 h. 5M TBHP in decane (15.73 mL, 78.63 mmol) was then added at 0° C. and the reaction mixture was stirred at rt for 4 h. The reaction was then quenched with saturated NaHSO₃ solution and extracted with EtOAc (2×150 mL). The combined organic layers were washed with saturated NaHSO₃ solution (2×100 mL), brine (2×100 mL), dried over Na₂SO₄, filtered, and concentrated under reduced pressure. The residue was purified by silica gel chromatography to afford ((2R,3S,5R)-5-(2-amino-6-oxo-1,6-dihydro-9H-purin-9-yl)-3-hydroxytetrahydrofuran-2-yl)methyl didecyl phosphate (A-12) (2.5 g, 15%) as a liquid.

To a stirred solution of ((2R,3S,5R)-5-(2-amino-6-oxo-1,6-dihydro-9H-purin-9-yl)-3-hydroxytetrahydrofuran-2-yl)methyl didecyl phosphate (A-12) (2 g, 3.18 mmol) in THE (40 mL) were added EDCI (1.83 g, 9.57 mmol), HOBt (1.29 g, 9.57 mmol), TEA (2.12 mL, 15.94 mmol) followed by tridecanoic acid (1.36 g, 6.38 mmol) at room temperature. The reaction mixture was stirred for 12 h at room temperature. The reaction was then quenched with ice cold water and extracted with EtOAc (2×100 mL). The organic layer washed with brine (100 mL), dried over Na₂SO₄, filtered, and concentrated under reduced pressure. The residue was purified by prep-TLC followed by silica gel chromatography (230-400 mesh) to afford (2R,3S,5R)-5-(2-amino-6-oxo-1,6-dihydro-9H-purin-9-yl)-2-(((bis(decyloxy) phosphoryl)oxy)methyl)tetrahydrofuran-3-yltridecanoate (Compound 8) (310 mg, 11%) as an off-white gum. MS (ESI) m/z 824.50[M+H]⁺; ¹H NMR (400 MHz, DMSO-d₆) δ 10.65 (s, 1H), 7.87 (s, 1H), 6.47 (br s, 2H), 6.14 (dd, J=8.4, 6.0 Hz, 1H), 5.34 (d, J=6.4 Hz, 1H), 4.22-4.11 (m, 3H), 3.95-3.88 (m, 4H), 2.86-2.83 (m, 1H), 2.46-2.44 (m, 1H) 2.34 (t, J=7.2 Hz, 2H), 1.55-1.50 (m, 6H), 1.45-1.24 (m, 46H), 0.84 (t, J=7.6 Hz, 9H).

### Example 32: Synthesis of Compound 9

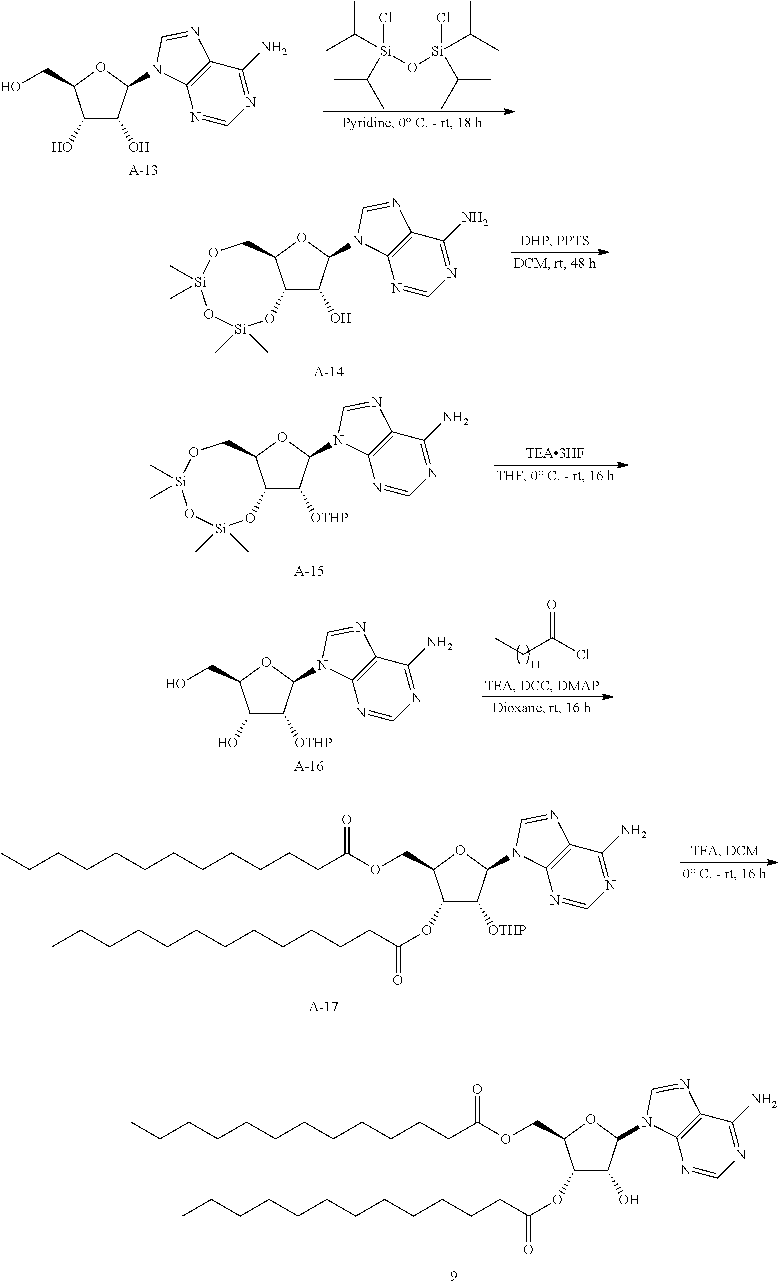

To A-13 (40 g, 149.67 mmol) in pyridine (400 mL, 10 vol) at 0° C. was added 1,3-dichloro-1,1,3,3-tetraisopropyldisiloxane (62.2 mL, 194.57 mmol). The reaction mixture was stirred at rt for 16 h. The solvent was then evaporated, and the residue was taken in water (250 mL) and extracted with EtOAc (2×500 mL). The combined organic layer was washed with water (250 mL), brine (250 mL), dried over anhydrous Na₂SO₄, filtered, and concentrated under reduced pressure. The residue was purified by silica gel (100-200 mesh) chromatography to afford (6aR,8R,9R,9aS)-8-(6-amino-9H-purin-9-yl)-2,2,4,4-tetraisopropyltetrahydro-6H-furo[3,2-f][1,3,5,2,4]trioxadisilocin-9-ol (A-14) (35 g, 46%) as a white solid.

To (6aR,8R,9R,9aS)-8-(6-amino-9H-purin-9-yl)-2,2,4,4-tetraisopropyltetrahydro-6H-furo[3,2-f][1,3,5,2,4]trioxadisilocin-9-ol (A-14) (20 g, 39.292 mmol) in DCM (400 mL, 20 vol) at rt was added PPTS (29.6 g, 117.87 mmol) followed by DHP (108 mL, 1178.76 mmol). The reaction mixture was stirred at rt for 48 h. The solvent was then evaporated, and the residue was taken in water (200 mL) and extracted with ethyl acetate (2×250 mL). The combined organic layers were washed with water (200 mL), brine (200 mL), dried over anhydrous Na₂SO₄, filtered, and concentrated under reduced pressure. The residue was purified by silica gel (100-200 mesh) chromatography to afford 9-((6aR,8R,9R,9aR)-2,2,4,4-tetraisopropyl-9-((tetrahydro-2H-pyran-2-yl)oxy)tetrahydro-6H-furo[3,2-f][1,3,5,2,4]trioxadisilocin-8-yl)-9H-purin-6-amine (A-15) (5 g, 21%) as a mixture of diastereomers.

To 9-((6aR,8R,9R,9aR)-2,2,4,4-tetraisopropyl-9-((tetrahydro-2H-pyran-2-yl)oxy)tetrahydro-6H-furo[3,2-f][1,3,5,2,4]trioxadisilocin-8-yl)-9H-purin-6-amine (A-15) (3.6 g, 6.07 mmol) in THE (36 mL, 10 vol) at 0° C. was added TEA.3HF (4.9 mL, 30.35 mmol). The reaction mixture was stirred at rt for 16 h. The solvent was then evaporated, and the residue was taken in water (50 mL) and extracted with MeOH: DCM (10:90) (4×50 mL). The combined organic layers were washed with brine (50 mL), dried over anhydrous Na₂SO₄, filtered, and concentrated under reduced pressure. The residue was triturated with Et₂O (2×10 mL) and pentane (2×10 mL) to afford (2R,3R,4R,5R)-5-(6-amino-9H-purin-9-yl)-2-(hydroxymethyl)-4-((tetrahydro-2H-pyran-2-yl)oxy)tetrahydrofuran-3-ol (A-16) (1.8 g, 85%) as a pale brown solid as a mixture of diastereomers.

To a stirred solution of (2R,3R,4R,5R)-5-(6-amino-9H-purin-9-yl)-2-(hydroxymethyl)-4-((tetrahydro-2H-pyran-2-yl)oxy)tetrahydrofuran-3-ol (A-16) (1.8 g, 5.12 mmol) in 1,4-dioxane (36 mL, 20 vol) at RT were added tridecanoic acid (6) (4.4 g, 20.51 mmol), TEA (3.6 mL, 25.64 mmol), DCC (3.2 g, 15.38 mmol) and DMAP (125 mg, 1.025 mmol). The reaction mixture was stirred at rt for 16 h. The solvent was then evaporated, and the residue was taken in water (50 mL) and extracted with ethyl acetate (3×50 mL). The combined organic layers were washed with water (50 mL), brine (50 mL), dried over anhydrous Na₂SO₄, filtered, and concentrated under reduced pressure. The crude residue was purified by silica gel (100-200 mesh) chromatography using MeOH: DCM (2:98) to afford (2R,3R,4R,5R)-5-(6-amino-9H-purin-9-yl)-4-((tetrahydro-2H-pyran-2-yl)oxy)-2-((tridecanoyloxy)methyl) tetrahydrofuran-3-yl tridecanoate (A-17) (1.7 g, 44%) as a pale brown liquid as a diastereomeric mixture.

To a stirred solution of (2R,3R,4R,5R)-5-(6-amino-9H-purin-9-yl)-4-((tetrahydro-2H-pyran-2-yl)oxy)-2-((tridecanoyloxy)methyl)tetrahydrofuran-3-yl tridecanoate (A-17) (1.5 g, 2.01 mmol) in DCM (30 mL, 20 vol) at 0° C. was added TFA (0.78 mL, 10.08 mmol). The reaction mixture was stirred at rt for 16 h. The solvent was then evaporated, and the residue was taken in water (25 mL), basified with aq. NaHCO₃ solution, and extracted with ethyl acetate (2×50 mL). The combined organic layer was washed with water (25 mL), brine (25 mL), dried over anhydrous Na₂SO₄, filtered, and concentrated under reduced pressure. The crude residue was purified by silica gel (100-200 mesh) chromatography and further purified by trituration with MeOH (2×25 mL) to afford (2R,3S,4R,5R)-5-(6-amino-9H-purin-9-yl)-4-hydroxy-2-((tridecanoyloxy)methyl) tetrahydrofuran-3-yl tridecanoate (Compound 9) (300 mg, 22%) as a pale green solid. MS (ESI) m/z 660.71 [M+H]⁺; H NMR (400 MHz, DMSO-d₆) δ 8.34 (s, 1H), 8.14 (s, 1H), 7.32 (br s, 2H), 5.89 (dd, J=14.4, 5.6 Hz, 2H), 5.31 (dd, J=5.2, 3.6 Hz, 1H), 5.02 (q, J=6.0 Hz, 1H), 4.35 (q, J=6.8 Hz, 1H), 4.28-4.22 (m, 2H), 2.39 (t, J=7.2 Hz, 2H), 2.30 (t, J=7.2 Hz, 2H), 1.60-1.45 (m, 4H), 1.21-1.17 (m, 36H), 0.85 (t, J=6.4 Hz, 6H).

### Example 33: Synthesis of Compound 10 and Compound 11

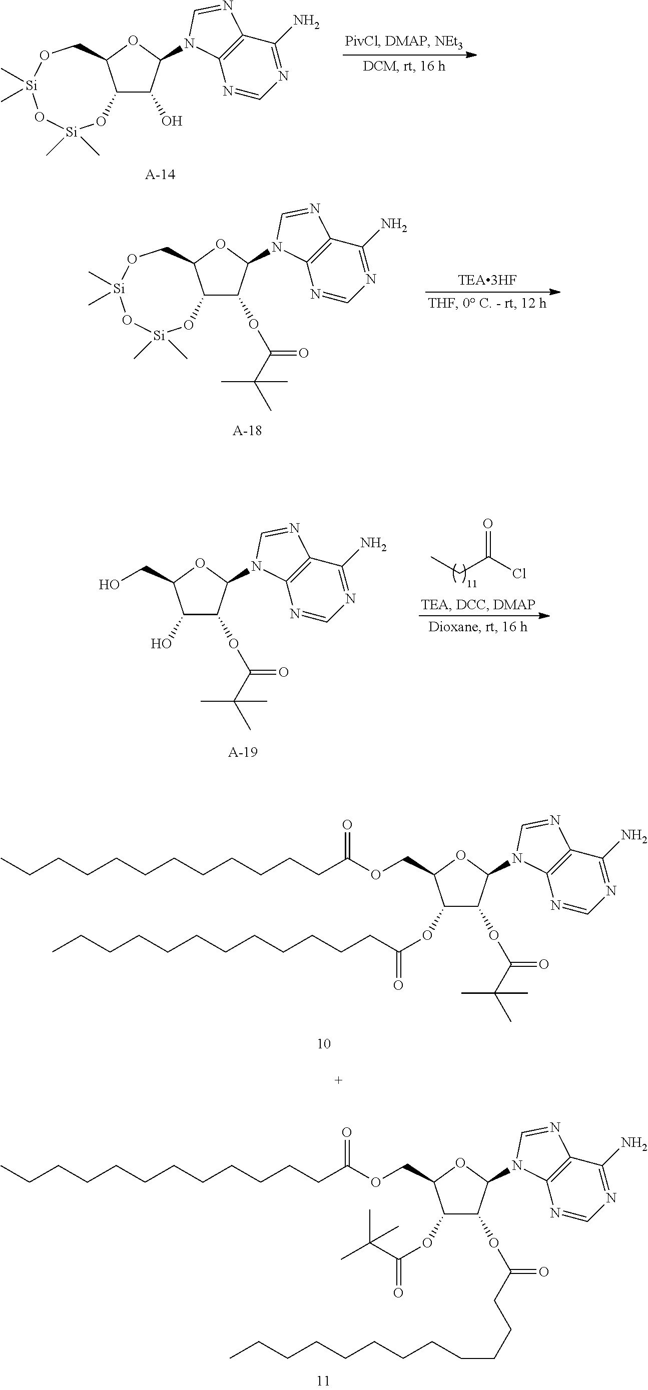

To a solution of (6aR,8R,9R,9aS)-8-(6-amino-9H-purin-9-yl)-2,2,4,4-tetraisopropyltetrahydro-6H-furo[3,2-f][1,3,5,2,4]trioxadisilocin-9-ol (A-14) (10 g, 19.646 mmol) in DCM (200 mL, 20 vol) at room temperature were added TEA (3.97 g, 39.29 mmol), DMAP (1.19 g, 9.825 mmol) followed by pivaloyl chloride (2.36 g, 19.646 mmol). The reaction mixture was stirred at rt for 48 h. The solvent was then evaporated, and the residue was taken in water (200 mL) and extracted with ethyl acetate (4×100 mL). The combined organic layers were washed with water (100 mL), brine (100 mL), dried over anhydrous Na₂SO₄, filtered, and concentrated under reduced pressure. The residue was purified by silica gel (100-200 mesh) chromatography to afford (6aR,8R,9R,9aR)-8-(6-amino-9H-purin-9-yl)-2,2,4,4-tetraisopropyltetrahydro-6H-furo[3,2-f]trioxadisilocin-9-yl pivalate (A-18) (8.5 g, 73%) as a white solid.

To a solution of (2R,3R,4R,5R)-2-(6-amino-9H-purin-9-yl)-4-hydroxy-5-(hydroxymethyl)tetrahydrofuran-3-yl pivalate (A-18) (5.3 g, 8.937 mmol) in THF (100 mL, 18 vol) at 0° C. was added TEA.3HF (7.34 mL, 44.68 mmol). The reaction mixture was stirred at rt for 16 h. The solvent was then evaporated, and the residue was triturated with Et₂O (2×30 mL) and pentane (2×30 mL) to afford (2R,3R,4R,5R)-2-(6-amino-9H-purin-9-yl)-4-hydroxy-5-(hydroxymethyl) tetrahydrofuran-3-yl pivalate (A-19) (2.3 g, 73%) as a white solid.

To (2R,3R,4R,5R)-2-(6-amino-9H-purin-9-yl)-4-hydroxy-5-(hydroxymethyl)tetrahydrofuran-3-yl pivalate (A-19) (2.3 g, 6.552 mmol) in DMF (100 mL, 43 vol) at rt were added tridecanoic acid (3) (7.02 g, 32.76 mmol), TEA (4.57 mL, 32.76 mmol), DCC (6.75 g, 32.76 mmol) and DMAP (800 mg, 6.552 mmol). The reaction mixture was stirred at rt for 16 h. The reaction mixture was taken in ice cooled water (200 mL) and extracted with ethyl acetate (4×100 mL). The combined organic layers were washed with ice cooled water (2×100 mL), brine (100 mL), dried over anhydrous Na₂SO₄, filtered, and concentrated under reduced pressure. The residue was purified by silica gel (100-200 mesh) chromatography and SFC to afford (2R,3R,4R,5R)-5-(6-amino-9H-purin-9-yl)-4-(pivaloyloxy)-2-((tridecanoyloxy) methyl) tetrahydrofuran-3-yl tridecanoate (Compound 10) (300 mg, 6%) as a pale yellow gum and Compound 11 as a pale gum. Compound 10: MS (ESI) m/z 744.67 [M+H]⁺; ¹H NMR (400 MHz, CDCl₃) δ 8.36 (s, 1H), 7.96 (s, 1H), 6.17 (d, J=4.8 Hz, 1H), 5.81 (t, J=5.6 Hz, 1H), 5.71-5.65 (m, 3H), 4.46-4.37 (m, 3H), 2.38-2.31 (m, 4H), 1.67-1.58 (m, 4H), 1.35-1.21 (m, 36H), 1.17 (s, 9H), 0.88 (t, J=6.0 Hz, 6H). Compound 11: MS (ESI) m/z 744.60 [M+H]⁺; ¹H NMR (400 MHz, CDCl₃) δ 8.37 (s, 1H), 7.94 (s, 1H), 6.15 (d, J=5.2 Hz, 1H), 5.94 (t, J=5.2 Hz, 1H), 5.66-5.61 (m, 3H), 4.45-4.37 (m, 3H), 2.42-2.28 (m, 4H), 1.74-1.62 (m, 2H), 1.60-1.54 (m, 4H), 1.30-1.16 (m, 45H), 0.87 (t, J=6.8 Hz, 6H).

### Example 34: Synthesis of Compound 12

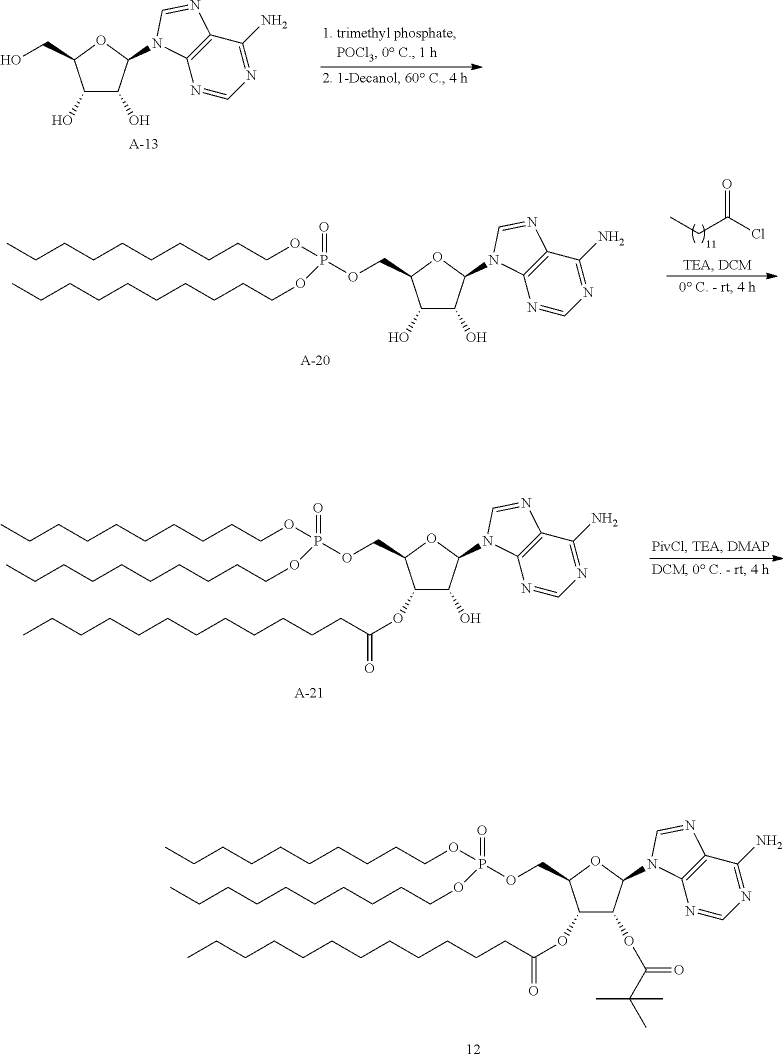

To A-13 (15 g, 56.179 mmol) in trimethyl phosphate (150 mL, 10 vol) at 0° C. was added POCl₃ (17.20 mL, 112.35 mmol). The reaction mixture was stirred at 0° C. for 1 h. 1-Decanol (75 mL, 5 vol) was then added and the reaction mixture was stirred at 60° C. for 4 h. The reaction was then quenched with water (250 mL) and extracted with EtOAc (2×500 mL). The combined organic layer was washed with water (250 mL), brine (250 mL), dried over anhydrous Na₂SO₄, filtered, and concentrated under reduced pressure. The residue was purified by silica gel (100-200 mesh) chromatography to afford ((2R,3S,4R,5R)-5-(6-amino-9H-purin-9-yl)-3,4-dihydroxytetrahydrofuran-2-yl)methyl didecyl phosphate (A-20) (12 g, 34%) as a pale yellow liquid.

To ((2R,3S,4R,5R)-5-(6-amino-9H-purin-9-yl)-3,4-dihydroxytetrahydrofuran-2-yl)methyl didecyl phosphate (A-20) (5 g, 7.97 mmol) in DCM (100 mL) was added TEA (3.35 mL, 23.91 mmol) at 0° C. The reaction mixture was stirred at 0° C. for 30 minutes. Tridecanoyl chloride (1.48 g, 6.37 mmol) was then added and the reaction mixture stirred for 4 h at rt. The solvent was then evaporated, and the residue was taken in water (200 mL) and extracted with DCM (2×100 mL). The combined organic layer was washed with brine (100 mL), dried over anhydrous Na₂SO₄, filtered, and concentrated under reduced pressure. The residue was purified by silica gel (100-200 mesh) chromatography to afford (2R,3S,4R,5R)-5-(6-amino-9H-purin-9-yl)-2-(((bis(decyloxy)phosphoryl)oxy)methyl)-4-hydroxytetrahydrofuran-3-yl tridecanoate (A-21) (mixture of regiomers) (1.5 g, 43%) as a pale yellow gum.

To (2R,3S,4R,5R)-5-(6-amino-9H-purin-9-yl)-2-(((bis(decyloxy)phosphoryl)oxy) methyl)-4-hydroxytetrahydrofuran-3-yl tridecanoate A-21 (mixture of regiomers) (950 mg, 0.115 mmol) in DCM (20 mL, 20 vol) was added Et₃N (0.32 mL, 0.23 mmol) followed by DMAP (70 mg, 0.057 mmol) at 0° C. The reaction mixture was stirred at 0° C. for 30 minutes. Pivaloyl chloride (3A) (139 mg, 0.115 mmol) was then added and reaction mixture was stirred at rt for 4 h. The reaction mixture was concentrated. The residue was taken in ice-cold water (100 mL) and extracted with DCM (2×100 mL). The combined organic layer was dried over anhydrous Na₂SO₄, filtered, and concentrated under reduced pressure. The residue was purified by silica gel (100-200 mesh) chromatography and further purified by SFC to afford (2R,3R,4R,5R)-5-(6-amino-9H-purin-9-yl)-2-(((bis(decyloxy)phosphoryl)oxy) methyl)-4-(pivaloyloxy)tetrahydrofuran-3-yl tridecanoate (Compound 12), (90 mg, 10%) as a liquid. MS (ESI) m/z 908.63 [M+H]⁺; H NMR (400 MHz, DMSO-d₆) δ 8.31 (s, 1H), 8.13 (s, 1H), 7.35 (br s, 2H), 6.19 (d, J=5.2 Hz, 1H), 5.89 (t J=5.6 Hz, 1H), 5.69 (t, J=5.2 Hz, 1H), 4.36 (d, J=4.4 Hz, 1H), 4.29-4.22 (m, 2H), 3.95-3.87 (m, 4H), 2.49-2.33 (m, 2H), 1.23-1.21 (m, 45H), 1.08 (s, 9H), 0.85-0.82 (m, 9H).

What is claimed is:

1. A composition comprising nanoparticles, wherein the nanoparticles comprise a prodrug of a nucleoside or nucleotide, and a pharmaceutically acceptable carrier; wherein the pharmaceutically acceptable carrier comprises albumin.

2. The composition of claim 1, wherein the nanoparticles have an average diameter of about 1000 nm or less for at least about 15 minutes after nanoparticle formation.

3. The composition of claim 1, wherein the nanoparticles have an average diameter of about 10 nm or greater for at least about 15 minutes after nanoparticle formation.

4. The composition of claim 1, the nanoparticles have an average diameter of from about 10 nm to about 1000 nm for at least about 15 minutes after nanoparticle formation.

5. The composition of claim 1, wherein the nanoparticles have an average diameter of about 1000 nm or less for at least about 4 hours after nanoparticle formation.

6. The composition of claim 1, wherein the nanoparticles have an average diameter of about 10 nm or greater for at least about 4 hours nanoparticle formation.

7. The composition of claim 1, the nanoparticles have an average diameter of from about 10 nm to about 1000 nm for at least about 4 hours after nanoparticle formation.

8. The composition of any one of claims 1-7, wherein the nanoparticles have an average diameter of from about 10 nm to about 1000 nm.

9. The composition of claim 8, wherein the nanoparticles have an average diameter of from about 30 nm to about 250 nm.

10. The composition of claim 9, wherein the nanoparticles have an average diameter of from about 50 nm to about 200 nm.

11. The composition of claim 10, wherein the nanoparticles have an average diameter of from about 50 nm to about 150 nm.

12. The composition of claim 11, wherein the nanoparticles have an average diameter of from about 50 nm to about 100 nm.

13. The composition of any one of claims 1-12, wherein the albumin is human serum albumin.

14. The composition of any one of claims 1-13, wherein the molar ratio of the prodrug of the nucleoside or nucleotide to pharmaceutically acceptable carrier is from about 1:1 to about 20:1.

15. The composition of claim 14, wherein the molar ratio of the prodrug of the nucleoside or nucleotide to pharmaceutically acceptable carrier is from about 2:1 to about 12:1.

16. The composition of any one of claims 1-15, wherein the nanoparticles are suspended, dissolved, or emulsified in a liquid.

17. The composition of any one of claims 1-16, wherein the composition is sterile filterable.

18. The composition of any one of claims 1-17, wherein the composition is dehydrated.

19. The composition of claim 18, wherein the composition is a lyophilized composition.

20. The composition of claim 18 or 19, wherein the composition comprises from about 0.9% to about 24% by weight of the prodrug of the nucleoside or nucleotide.

21. The composition of claim 20, wherein the composition comprises from about 1.8% to about 16% by weight of the prodrug of the nucleoside or nucleotide.

22. The composition of any one of claims 18-21, wherein the composition comprises from about 76% to about 99% by weight of the pharmaceutically acceptable carrier.

23. The composition of claim 22, wherein the composition comprises from about 84% to about 98% by weight of the pharmaceutically acceptable carrier.

24. The composition of any one of claims 18-23, wherein the composition is reconstituted with an appropriate biocompatible liquid to provide a reconstituted composition.

25. The composition of claim 24, wherein the appropriate biocompatible liquid is a buffered solution.

26. The composition of claim 24, wherein the appropriate biocompatible liquid is a solution comprising dextrose.

27. The composition of claim 24, wherein the appropriate biocompatible liquid is a solution comprising one or more salts.

28. The composition of claim 24, wherein the appropriate biocompatible liquid is sterile water, saline, phosphate-buffered saline, 5% dextrose in water solution, Ringer's solution, or Ringer's lactate solution.

29. The composition of any one of claims 24-28, wherein the nanoparticles have an average diameter of from about 10 nm to about 1000 nm after reconstitution.

30. The composition of claim 29, wherein the nanoparticles have an average diameter of from about 30 nm to about 250 nm after reconstitution.

31. The composition of claim 30, wherein the nanoparticles have an average diameter of from about 50 nm to about 200 nm after reconstitution.

32. The composition of claim 31, wherein the nanoparticles have an average diameter of from about 50 nm to about 150 nm after reconstitution.

33. The composition of claim 32, wherein the nanoparticles have an average diameter of from about 50 nm to about 100 nm after reconstitution.

34. The composition of any one of claims 1-33, wherein the composition is suitable for injection.

35. The composition of any one of claims 1-34, wherein the composition is suitable for intravenous administration.

36. The composition of any one of claims 1-33, wherein the composition is administered intraperitoneally, intraarterially, intrapulmonarily, orally, by inhalation, intravesicularly, intramuscularly, intratracheally, subcutaneously, intraocularly, intrathecally, intratumorally, or transdermally.

37. The composition of any one of claims 1-36, wherein the prodrug of the nucleoside or nucleotide is a prodrug of a nucleoside.

38. The composition of any one of claims 1-36, wherein the prodrug of the nucleoside or nucleotide is a prodrug of a nucleotide.

39. The composition of any one of claims 1-38, wherein the prodrug of the nucleoside or nucleotide comprises a cyclic or acyclic sugar moiety.

40. The composition of any one of claims 1-38, wherein the prodrug of the nucleoside or nucleotide comprises a sugar moiety that is an optionally substituted pentose.

41. The composition of any one of claims 1-38, wherein the prodrug of the nucleoside or nucleotide comprises a sugar moiety that is a modified pentose moiety.

42. The composition of any one of claims 1-38, wherein the prodrug of the nucleoside or nucleotide comprises a nitrogenous base that is an optionally substituted purine-base or an optionally substituted pyrimidine-base.

43. The composition of any one of claims 1-42, wherein the prodrug of the nucleoside or nucleotide is derived from a nucleoside or nucleotide that is an anticancer agent.

44. The composition of any one of claims 1-42, wherein the prodrug of the nucleoside or nucleotide is derived from a nucleoside or nucleotide that is an antiviral agent.

45. A method of treating a disease in a subject in need thereof comprising administering the composition of any one of claims 1-44.

46. The method of claim 45, wherein the disease is cancer.

47. The method of claim 45, wherein the disease is caused by an infection.

48. The method of claim 47, wherein the infection is viral.

49. A method of delivering a prodrug of a nucleoside or nucleotide to a subject in need thereof comprising administering the composition of any one of claims 1-44.

50. A process of preparing a composition of any one of claims 1-44 comprising
a) dissolving a prodrug of a nucleoside or nucleotide in a volatile solvent to form a solution comprising a dissolved prodrug of a nucleoside or nucleotide;
b) adding the solution comprising the dissolved prodrug of a nucleoside or nucleotide to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form the composition of any one of claims 1-44.

51. The process of claim 50, wherein the volatile solvent is a chlorinated solvent, alcohol, ketone, ester, ether, acetonitrile, or any combination thereof.

52. The process of claim 51, wherein the volatile solvent is chloroform, ethanol, methanol, or butanol.

53. The process of any one of claims 50-52, wherein the homogenization is high pressure homogenization.

54. The process of claim 53, wherein the emulsion is cycled through high pressure homogenization for an appropriate amount of cycles.

55. The process of claim 54, wherein the appropriate amount of cycles is from about 2 to about 10 cycles.

56. The process of any one of claims 50-55, wherein the evaporation is accomplished with a rotary evaporator.

57. The process of any one of claims 50-56, wherein the evaporation is under reduced pressure.

58. The composition of any one of claims 1-36, wherein the prodrug of the nucleoside or nucleotide is not a gemcitabine derivative having the following structure:

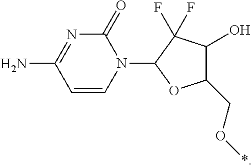

59. The composition of any one of claims 1-36, wherein the prodrug of the nucleoside or a nucleotide is not a compound of Formula (I):
A¹-X¹—X²-A² Formula (I)
wherein:
A¹ is a hydrophilic group;
A² is a gemcitabine moiety;
X¹ is —(CH₂)₁₂—, —(CH₂)₁₄—, —(CH₂)₁₆—, —(CH₂)₁₈—, —(CH₂)₂₀—, or —(CH₂)₂₂—; and
X² is a direct bond or an organic group.

60. The composition of claim 59, wherein A¹ is a carboxylic acid group, a carboxylate anion, or a carboxylate ester.
